(19) United States
(12) Patent Application Publication
(10) Pub. No.: US 2007/0185079 A1
(43) Pub. Date: Aug. 9, 2007

(54) Therapeutic agents I

(75) Inventors: Emma Evertsson, Molndal (SE); Tord Inghardt, Molndal (SE); Jan Lindberg, Molndal (SE); Anna Linusson, Umea (SE); Fabrizio Giordanetto, Molndal (SE)

Correspondence Address: Pepper Hamilton LLP, 500 Grant Street, One Mellon Bank Center, 50th Floor, Pittsburgh, PA, 15219-2502, US

(73) Assignee: ASTRAZENECA AB, Sodertalje (SE)

(21) Appl. No.: 10/596,994

(22) PCT Filed: Jan. 5, 2005
(86) PCT No.: PCT/SE05/00004

§ 371 Date: Nov. 22, 2006

(30) Foreign Application Priority Data
Nov. 16, 2004 (GB) 0425209.4
Jan. 7, 2004 (GB) 0400196.2

(51) Int. Cl. A61K 31/554

(52) U.S. Cl. 514/211.13

(57) ABSTRACT

Compounds of formula(I), processes for preparing such compounds, their use in the treatment of obesity, psychiatric disorders, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, and pharmaceutical compositions containing them.

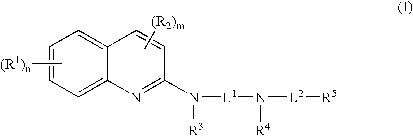

### FIELD OF INVEVTION

The present invention relates to certain N-cycloalkyl, aryl or heteroaryl N′-quinolin-2-yl cycloalkyldiamines of formula I, to processes for preparing such compounds, to their use in the treatment of obesity, psychiatric and neurological disorders, and to pharmaceutical compositions containing them.

### BACKGROUND OF THE INVENTION

Melanin concentrating hormone (MCH) is a cyclic peptide that was first isolated from fish over 15 years ago. In mammals, MCH gene expression is localised to the ventral aspect of the zona inserta and the lateral hypothalamic area (Breton et al., Molecular and Cellular Neurosciences, vol. 4, 271-284 (1993)). The latter region of the brain is associated with the control of behaviours such as eating and drinking, with arousal and with motor activity (Baker, B., Trends Endocrinol. Metab. 5: 120-126(1994), vol. 5, No. 3, 120-126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterised by compulsive eating and excessive body weight. MCH projections are found throughout the brain, including the spinal cord, an area important in processing nociception, indicates that agents acting through MCH1r, such as compounds of formula I, will be useful in treating pain.

Two receptors for MCH (MCH1r (Shimomura et al. Biochem Biophys Res Commun 1999 Aug. 11; 261(3):622-6) & MCH2r (Hilol et al. J Biol Chem. 2001 Jun. 8; 276(23):20125-9)) have been identified in humans, while only one (MCH1r) is present in rodent species (Tan et al. Genomics. 2002 June; 79(6):785-92). In mice lacking MCH1r, there is no increased feeding response to MCH, and a lean phenotype is seen, suggesting that this receptor is responsible for mediating the feeding effect of MCH (Marsh et al. Proc Natl Acad Sci USA. 2002 Mar. 5; 99(5):3240-5). In addition, MCH receptor antagonists have been demonstrated to block the feeding effects of MCH (Takekawa et al. Eur J Pharmacol. 2002 Mar. 8; 438(3):129-35), and to reduce body weight & adiposity in diet-induced obese rats (Borowsky et al. Nat Med. 2002 August; 8(8):825-30). The conservation of distribution and sequence of MCH1r suggest a similar role for this receptor in man and rodent species. Hence, MCH receptor antagonists have been proposed as a treatment for obesity and other disorders characterised by excessive eating and body weight.

U.S. Pat. No. 3,020,283 discloses that certain N,N′-bis lepid-2-yl 1,x-diamino C₁₋ₓ alkanes where x is an integer from 2 to 12 and N,N′-bis lepid-2-yldiaminocycloalkanes are useful as anthelmintics.

U.S. Pat. No. 5,093,333 discloses certain N-substituted (cyclicaminoalkyl) 2-aminoquinolines which are useful for treating hypofunction of the cholinergic system and therefore useful in treating dementias involving the cholinergic system.

U.S. Pat. No. 4,203,988 discloses certain pyridinyl and quinolinyl ureas which are useful in treating gastric secretion.

WO99/55677 discloses 2-(aminoalkylamino)quinolin-4-ones which are useful as anti-bacterial agents.

WO02/58702 discloses substituted 2-(aminoalkyl amino) quinolines which are antagonists of urotensin II which are alleged to be useful in treating cardiovascular diseases characterised by excessive or abnormal vasoconstriction and myocardial dysfunction and also in diseases of the CNS for example addiction, schizophrenia, anxiety and depression and metabolic diseases such as diabetes.

1,4-Anhydro-2,3,5-trideoxy-3-[[(3,4-dichlorophenyl)methyl]amino]-5-[(4-ethoxy-2-quinolinyl)amino]-D-erythro-pentitol is disclosed as an intermediate in Bioorg. Med. Chem. Lett. 13, 1265-68 (2003),

WO02/096911 discloses heteroaryl diazabicycloalkanes as modulators of the nicotinic receptor and/or monoamine receptors which are useful as central nervous system modulators. Co-pending application WO 2004/087669 discloses that compounds of formula ii
Q-L-Y—R₁ ii
in which Q is optionally substituted 2-quinolyl, tetrahydroquinazolinyl or pyrimidyl, L is inter alia 1,4-diaminocyclohexyl or 1,3-Diaminocyclopentyl, Y is a bond, methylene, carbonyl, or sulphonyl, and R₁ is inter alia aryl or heteroaryl are MCH receptor antagonists. Co-pending application PCT/GB03/02884 (WO2004/004726) discloses compounds of the general formula (I) 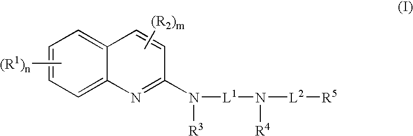
wherein
R¹ represents a C₁₋₄alkoxy group optionally substituted by one or more fluoro or a C₁₋₄alkyl group optionally substituted by one or more fluoro;
n represents 0 or 1;
R² represents a C₁₋₄alkyl group optionally substituted by one or more fluoro or a C₁₋₄alkoxy group optionally substituted by one or more fluoro;
m represents 0 or 1;
R³ represents H or a C₁₋₄alkyl group;
L¹ represents an alkylene chain (CH₂)ᵣ in which r represents 2 or 3 or L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group and additionally when R⁵ represents 9,10-methanoanthracen-9(10H)-yl the group -L′-N(R⁴)— together represents a piperidyl ring which is linked to L² through the piperidinyl nitrogen and to N—R³ via the 4 position of the piperidyl ring with the proviso that when R⁵ represents 9,10-methanoanthracen-9(10H)-yl then r is only 2;
R⁴ represents H or a C₁₋₄alkyl group optionally substituted by one or more of the following: an aryl group or a heteroaryl group;
L² represents a bond or an alkylene chain (CH₂)ₛ in which s represents 1, 2 or 3 wherein the alkylene chain is optionally substituted by one or more of the following: a C₁₋₄alkyl group, phenyl or heteroaryl;
R⁵ represents aryl (wherein aryl means phenyl, naphthyl, or 9,10-methanoanthracen-9(10H)-yl, each of which is optionally substituted by one or more of the following: halo, a C₁₋₄alkyl group, phenyl, or a group of formula NR⁶R⁷ wherein R⁶ and R⁷ are independently selected from H or a C₁₋₄alkyl group), a heterocyclic group (wherein the term “heterocyclic group” as used herein means thienyl, firyl, pyridyl, pyrrolyl, quinolinyl, indolyl, benzofuranyl or benzo[b]thienyl each of which is optionally substituted by one or more of the following: halo, a C₁₋₄alkyl group, a C₁₋₄acyl group or nitro) or a C₃₋₈cycloalkyl group which is optionally fused to a phenyl or to a heteroaryl group (wherein the term “heteroaryl” means thienyl, furyl or pyrrolyl);
as well as optical isomers and racemates thereof as well as pharmaceutically acceptable salts, thereof;
with a first proviso that when n is 0, and m is 1 and R² is methyl located at the 4-position of the quinoline ring, and R³ is H and R⁴ is H and L¹ is (CH₂)₂ or (CH₂)₃ or 1,4-cyclohexyl, and L² is a bond then R⁵ is not 4-methylquinolin-2-yl;
and with a second proviso that when n is 0, and m is 0 or 1 and R² is a C₁₋₃alkoxy group located at the 4-position of the quinoline ring, and R³ is H or a C₁₋₃alkyl group and R⁴ is H or a C₁₋₃alkyl group and L¹ is (CH₂)₃ and L² is methylene optionally substituted by one or more C₁₋₃alkyl groups or phenyl then R⁵ is not phenyl, thienyl or indolyl optionally substituted by one, two or three C₁₋₄alkyl groups or halo which are MCH1r antagonists which are useful in treating obesity and related disorders, psychiatric disorders, neurological disorders and pain. The compounds claimed and disclosed in this application are disclaimed from the present invention.

There is an unmet need for MCH receptor antagonists that are more potent, more selective, more bioavailable and less toxic than known compounds in this field. The present invention provides additional compounds that are MCH1r antagonists which are useful in treating obesity and related disorders, psychiatric disorders, neurological disorders and pain.

### DESCRIPTION OF THE INVENTION

The invention relates to compounds of the general formula (I) 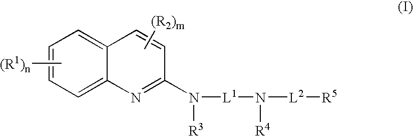
wherein
R¹ represents a C₁₋₄ alkoxy group optionally substituted by one or more fluoro, a C₁₋₄ alkyl group optionally substituted by one or more fluoro, halo, cyano, a group OSO₂C₁₋₄alkyl wherein the alkyl group is optionally substituted with one or more fluorine atoms, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring,
n represents 0, 1, 2 or 3;
R² represents a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H or a C₁₋₄ alkyl group;
L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively;
R⁴ represents H or a C₁₋₄ alkyl group optionally substituted by one or more of the following: fluoro or C₁₋₄ alkoxy optionally substituted by one or more fluoro;
L² represents an alkylene chain (CH₂)ₛ in which s represents 1, 2 or 3 wherein the alkylene chain is optionally substituted by one or more of the following: fluoro or C₁₋₄ alkyl;
or L² may also represent a 5-6 membered carbocyclic ring fused to R⁵;
R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridinyl, 5H-pyrrolo[2,3-b]pyrazinyl, 1H-pyrrolo[3,2-c]pyridinyl, 1H-pyrrolo[2,3-c]pyridinyl, 1H-pyrrolo[2,3-b]pyridinyl, 1H-indazolyl, 1H-pyrrolo[3,2-h]quinolinyl, 1H-pyrrolo[3,2-b]pyridinyl, 2,1,3-benzothiadiazolyl, 2,1,3-benzoxadiazolyl, quinazolinyl or triazolyl wherein each R⁵ is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group O_{z}(CH₂)_{w}R^{z} in which z and w independently are 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro;
as well as optical isomers and racemates thereof as well as pharmaceutically acceptable salts, thereof;
with the proviso that when
R¹ represents a C₁₋₄alkoxy group optionally substituted by one or more fluoro or a C₁₋₄alkyl group optionally substituted by one or more fluoro; and
n represents 0 or 1; and
R² represents a C₁₋₄alkyl group optionally substituted by one or more fluoro or a C₁₋₄alkoxy group optionally substituted by one or more fluoro ; and
m represents 0 or 1; and
R³ represents H or a C₁₋₄alkyl group; and
L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group; and
L² represents an alkylene chain (CH₂)ₛ in which s represents 1, 2 or 3 wherein the alkylene chain is optionally substituted by one or more of the following: a C₁₋₄alkyl group; and
R⁵ represents aryl wherein aryl means phenyl or naphthyl each of which is optionally substituted by one or more of the following: halo, a C₁₋₄alkyl group or phenyl, or
R⁵ represents a heterocyclic group wherein the term heterocyclic group means thienyl, furyl, pyridyl, pyrrolyl, quinolinyl, indolyl, benzofuranyl or benzo[b]thienyl each of which is optionally substituted by one or more of the following: halo or a C₁₋₄alkyl group;
or L² represents a C₅₋₆cycloalkyl group which is fused to an R⁵ which is phenyl or a heteroaryl group selected from thienyl, furyl or pyrrolyl;
then R⁴ does not represent H or a C₁₋₄alkyl group; and excluding 1,4-anhydro-2,3,5-trideoxy-3-[[(3,4-dichlorophenyl)methyl]amino]-5-[(4-ethoxy-2-quinolinyl)amino]-D-erythro-pentitol.

A compound of formula I 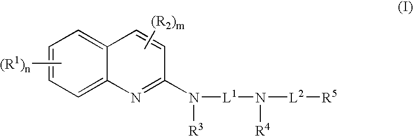
wherein
R¹ represents a C₁₋₄ alkoxy group optionally substituted by one or more fluoro, a C₁₋₄ alkyl group optionally substituted by one or more fluoro, halo, cyano, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring,
n represents 0, 1, 2 or 3;
R² represents a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H or a C₁₋₄ atkyl group;
L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively or alternatively the group —N(R³)-L¹-N(R⁴) together represents a saturated heterocyclic ring containing from 6 to 9 carbon atoms and the nitrogens bearing R³ and R⁴ which is bicyclic;
R⁴ represents H or a C₁₋₄ alkyl group optionally substituted by one or more of the following: fluoro or C₁₋₄ alkoxy optionally substituted by one or more fluoro;
² represents an alkylene chain (CH₂)ₛ in which s represents 1, 2 or 3 wherein the alkylene chain is optionally substituted by one or more of the following: fluoro or C₁₋₄ alkyl;
or L² may also represent a 5-6 membered carbocyclic ring fused to R⁵,
R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole wherein each R⁵ is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more of the following:cyano, halo, a C₁₋₄ alkyl group optionally substituted by one or more fluoro, or a C₁₋₄alkoxy group optionally substituted by one or more fluoro;
as well as optical isomers and racemates thereof as well as pharmaceutically acceptable salts, thereof;
with the proviso that when
Rrepresents a C₁₋₄alkoxy group optionally substituted by one or more fluoro or a C₁₋₄alkyl group optionally substituted by one or more fluoro; and
n represents 0 or 1; and
R² represents a C₁₋₄alkyl group optionally substituted by one or more fluoro or a C₁₋₄alkoxy group optionally substituted by one or more fluoro ; and
m represents 0 or 1; and
R³ represents H or a C₁₋₄alkyl group; and
L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group; and
L² represents an alkylene chain (CH₂)ₛ in which s represents 1, 2 or 3 wherein the alkylene chain is optionally substituted by one or more of the following: a C₁₋₄alkyl group; and
R⁵ represents aryl wherein aryl means phenyl or naphthyl each of which is optionally substituted by one or more of the following: halo, a C₁₋₄alkyl group or phenyl, or
R⁵ represents a heterocyclic group wherein the term heterocyclic group means thienyl, furyl, pyridyl, pyrrolyl, quinolinyl, indolyl, benzofuranyl or benzo[b]thienyl each of which is optionally substituted by one or more of the following: halo or a C₁₋₄alkyl group;
or L² represents a C₅₋₆cycloalkyl group which is fused to an R⁵ which is phenyl or a heteroaryl group selected from thienyl, furyl or pyrrolyl;
then R⁴ does not represent H or a C₁₋₄alkyl group.

Particular groups now follow in which some of R¹, R², R³, R⁴, R⁵, L¹, L², n and m in compounds of formula I are further defined. It will be understood that such group definitions may be used where appropriate with any of the other group definitions, claims or embodiments defined hereinbefore or hereinafter.

In a particular group of compounds of formula I, n is 1 and R¹ represents methoxy, fluoro, chloro or dimethylamino. In particular R¹ is attached at either the 6 or 7 position of the quinoline ring. In particular when n is 2, R¹ is independently selected from methoxy, fluoro, chloro or dimethylamino and and is attached at the 6 and 7 position.

In a particular group of compounds of formula I, L¹ represents a monocyclic —(CH₂)ₚC₅₋₆ (CH₂)_{q}— cycloalkyl group in which p and q are independently 0 or 1 and wherein there are 3 carbon atoms between the two nitrogens bearing R³ and R⁴, respectively, wherein one of the carbons of the cycloalkyl group may be replaced by O or the group —N(R³)-L¹-, or the group L¹-N(R⁴), together represent a saturated heterocyclic ring containing from 4 to 6 carbon atoms and the nitrogen bearing R³ or R⁴ respectively.

Particularly in compounds of formula I, p is 0, q is 0 and L¹ is 1,3-cyclopentyl.

Particularly in compounds of formula I, p is 0, q is 0 and L¹ is 1,3-cyclohexyl.

Particularly in compounds of formula I, p is 1, q is 0 and L¹ is —CH₂(1,2-cyclopentyl)-.

Particularly in compounds of formula I, p is 0, q is 1 and L¹ is -(1,2-cyclopentyl)CH₂—.

In a particular group of compounds of formula I, R⁵ represents a heterocyclic group selected from imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole wherein each R⁵ is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C¹⁻⁴alkyl group optionally substituted by one or more fluoro or a C₁₋₄alkoxy group optionally substituted by one or more fluoro, or by a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more of the following:cyano, halo, a C₁₋₄ alkyl group optionally substituted by one or more fluoro, or a C₁₋₄alkoxy group optionally substituted by one or more fluoro.

A further particular group of compounds of formula I, is represented by formula IA 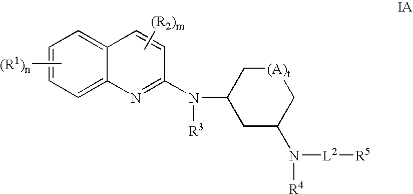
in which
R¹ represents chloro, fluoro, methoxy or a group NR^{a}R^{b} in which R^{a} and R^{b} independently represent H or a C₁₋₄alkyl group.
n represents 0 or 1, 2 and when n=1 the substituent is attached to either position 6 or 7;
R² represents a C₁₋₄alkyl group or a C₁₋₄alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H;
A represents CH₂ and t is O or 1;
R⁴represents H;
L² represents CH₂, C(CH₃)₂ or CF₂; and
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro as well as optical isomers and racemates thereof as well as pharmaceutically acceptable salts thereof.

Another particular group of compounds of formula I is represented by formula IB 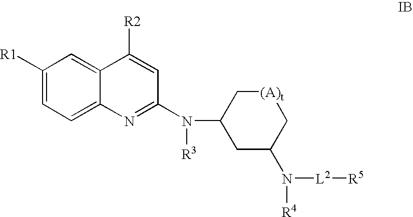
in which
R¹ represents H, methoxy, dimethylamino, chloro or fluoro;
R² represents H, a C₁₋₄alkyl group or a C₁₋₄ alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with is the nitrogen atom to which they are attached represent a saturated 3 to 7 membered heterocyclic ring;
R³represents H;
A represents CH₂ and t is 0 or 1;
R⁴ represents H;
L² represents CH₂, C(CH₃)₂ or CF₂; and
R⁵ represents 2-thienyl, 3-thienyl, indol-3-yl, 2-pyrrolyl, 5-pyrimidinyl, 4-thiadiazolyl, pyrazolyl, or quinolin-2-yl each of which is optionally substituted by one or more of the following: cyano, halo, a C₁₋₄ alkyl group optionally substituted by one or more fluoro, a C₁₋₄ alkoxy group optionally substituted by one or more fluoro and in addition when R⁵ is 2-thienyl it is optionally additionally substituted by pyridyl, 2-thienyl or 3-pyrazolyl each of which is optionally substituted by halo or a C₁₋₄ alkyl group optionally substituted by one or more fluoro and when R⁵ is indol-3-yl it is optionally additionally substituted by 1-(thiazol-5-yl) methyl which is optionally substituted by halo.

Yet another particular group of compounds of formula I is represented by formula IC 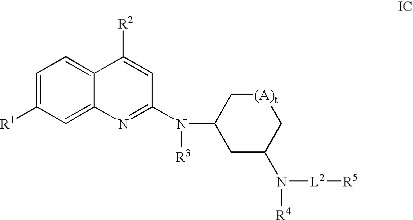
in which
R¹ represents H, methoxy, dimethylamino, chloro or fluoro;
R² represents H, a C₁₋₄alkyl group or a C₁₋₄ alkoxy group optionally substituted by one or more fluoro, a group NR^{a}R^{b} in which R^{a} and R^{b} independently represent H or a C₁₋₄alkyl group or R^{a} and R¹ together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³represents H;
A represents CH₂ and t is 0 or 1;
R⁴ represents H;
L² represents CH₂, C(CH₃)₂ or CF₂; and
R⁵ represents 2-thienyl, 3-thienyl, 1H-indol-3-yl, 2-pyrrolyl, 5-pyrimidinyl, 4-thiadiazolyl, pyrazolyl, 1H-pyrrolo[3,2-b]pyridinyl or quinolin-2-yl each of which is optionally substituted by one or more of the following: cyano, halo, a C₁₋₄ alkyl group optionally substituted by one or more fluoro, a C₁₋₄ alkoxy group optionally substituted by one or more fluoro and in addition when R⁵ is 2-thienyl it is optionally additionally substituted by pyridyl, 2-thienyl or 3-pyrazolyl each of which is optionally substituted by halo or a C₁₋₄ alkyl group optionally substituted by one or more fluoro and when R⁵ is indol-3-yl it is optionally additionally substituted by 1-(thiazol-5-yl) methyl which is optionally substituted by halo.

Particularly in compounds of formulae I, IA , IB and IC the two nitrogen atoms are in a trans orientation on the cycloalkyl ring.

More particularly in compounds of formulae I, IA, IB and IC the absolute configuration of the cycloalkyl carbon atoms to which the nitrogen atoms are attached is S, S.

In a particular group of compounds of formulae I, IA, IB and IC, R¹, R², R³, R⁴, R⁴, R⁵ and L², n and m are as listed in any definition of these substituents in this specification and L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively; with the proviso that L¹ is not 1,4-cyclohexyl or 1,3 cyclopentyl.

Particularly in compounds of formula I, L¹ is selected from:

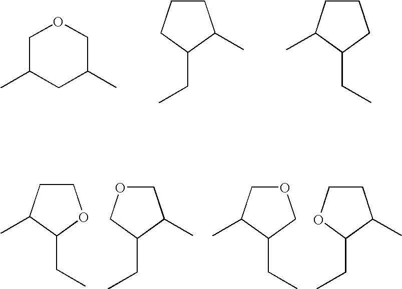

It will be understood that in the above the free bond to the left of the page is attached to the nitrogen bearing R³ and the free bond to the right of the page is attached to the nitrogen bearing R⁴. For the avoidance of doubt when Q represents 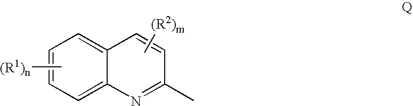
particular compounds of the invention are 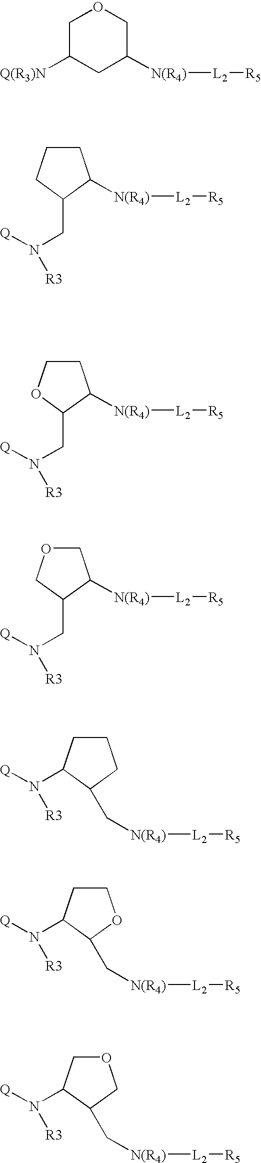
in which Q, R³, L² and R⁵ are as previously defined.

In a particular group of compounds of formula I, L¹ represents a (CH₂)ₚC₇₋₁₀ cycloalkyl group in which p is 0 or 1 and in which the cycloalkyl group is fused or bridged bicyclic provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³or R⁴ respectively and R¹, R², R³, R⁴, R⁵, L², m and n are as defined above.

Examples where Lis bicyclic include particularly

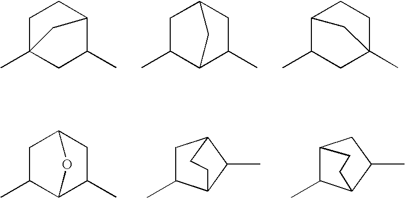

Examples where —N(R³)-L¹- together represents a saturated heterocyclic ring containing from 6 to 9 carbon atoms and the nitrogen bearing R³ include

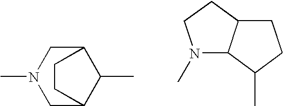

Examples where the group -L¹-N(R⁴)- together represents a heterocyclic ring containing from 2 to 9 carbon atoms and the nitrogen bearing R⁴ include:

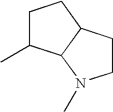

It will be understood that in the above the free bond to the left of the page is attached to the nitrogen bearing R³ (or to the quinoline ring) and the free bond to the right of the page is attached to the nitrogen bearing R⁴ (or to L2). For the avoidance of doubt when Q represents

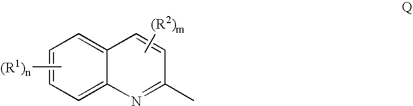

Examples of compounds where L¹ is bicyclic include 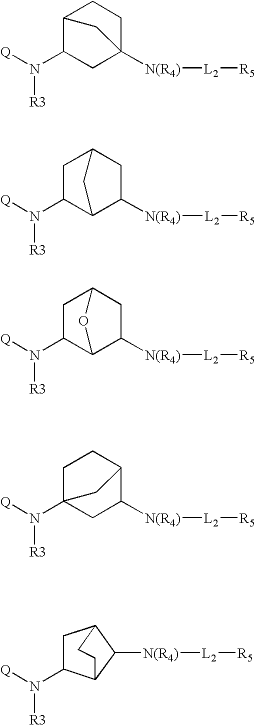
in which Q, R³, R⁴, L² and R⁵ are as previously defined.

Examples of compounds where —N(R³)-L¹- together represents a saturated heterocyclic ring containing from 6 to 9 carbon atoms and the nitrogen bearing R³ include 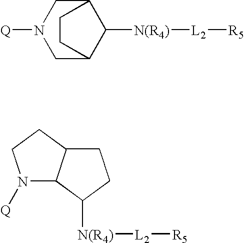
in which Q, R³, R⁴, L² and R⁵ are as previously defined.

Examples of compounds where -L¹-N(R⁴)- together represent a saturated heterocyclic ring containing from 6 to 9 carbon atoms and the nitrogen bearing R⁴ include compounds of formula 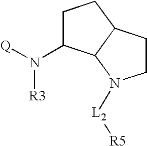
in which Q, R³, R⁴, L² and R⁵ are as previously defined.

Further particular values of R¹, R², R³, R⁴, R⁵, L¹, L², n and m in compounds of formula I now follow. It will be understood that such values may be used where appropriate with any of the definitions, claims or embodiments defined hereinbefore or hereinafter.

Particularly R¹ represents H, methoxy, fluoro, chloro or dimethylamino.

Particularly R² represents H, methyl, methoxy, dimethylamino or N,N-dimethylcarbamoyl.

Particularly R⁵ represents one of the following: 3-thienyl, 1-methylpyrrol-2-yl, 1-methylindol-3-yl, 2,4-dimethoxypyrimidin-5-yl, 2-(phenylsulfonyl)-1,3-thiazol-5-yl, 1-methyl-5-(trifluoromethyl)-1H-pyrazol-3-yl]-2-thienyl, 1-[(2-chloro-1,3-thiazol-5-yl)methyl]-1H-indol-3-yl}, 5-(2-thienyl)thien-2-yl, 5-pyridin-2-yl-2-thienyl, 1,2,3-thiadiazol-4-yl, 4-chloro-1-methyl-1H-pyrazol-3-yl and quinolin-2-yl. Particularly R⁵ also represents one of the following: 1-[3-(trifluoromethyl)pyridin-2-yl]-1H-indol-3-yl, 6-cyano-1-methylindol-3-yl, 1-methyl-1H-indazol-3-yl, 1-methyl-1H-pyrrolo[2,3-b]pyridin-3-yl, 1-methyl-1H-indol-2-yl, 1-[3-(trifluoromethyl)pyridin-2-yl]-1H-indol-3-yl, 1-[4-(trifluoromethyl)phenyl]-1H-pyrrol-3-yl, 5-difluoromethoxy-1H-indol-3-yl, 1-methyl-1H-pyrrolo[3,2-h]quinolin-3-yl), 1-methyl-1H-pyrrolo[3,2-b]pyridin-3-yl, 1-methyl-1H-pyrrolo[2,3-c]pyridin-3-yl, 5-(benzyloxy)-1-methyl-1H-indol-3-yl, imidazo[1,2-a]pyridin-3-yl, quinolin-3-yl, 2-bromo-4-methoxyphenyl, 1,3-dimethyl-1H-pyrazol-4-yl)methyl, and 2,1,3-benzothiadiazol-4-yl.

In nine particular groups of compounds of formula I, R⁵ represents one of the following: 1H-pyrrolo[3 ,2-c]pyridinyl;
1H-pyrrolo[2,3-b]pyridinyl;
1H-indazolyl;
1-imidazo[1,2-a]pyridinyl;
5H-pyrrolo[2,3-b]pyrazinyl;
1H-pyrrolo[3,2-b]pyridinyl;
1H-pyrrolo[3,2-h]quinolinyl;
2,1,3-benzothiadiazolyl; and
2,1,3-benzoxadiazolyl;
wherein each of these heterocycles is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group O_{z}(CH₂)_{w}R^{z} in which z and w independently are 0 or 1 and R^{z} represents phenyl or a heterocyclic group selected from thienyl, pyridyl, thiazolyl, pyrazolyl, wherein each R^{z} is optionally substituted by one or more of the following:cyano, halo, a C₁₋₄ alkyl group optionally substituted by one or more fluoro, or a C₁₋₄alkoxy group optionally substituted by one or more fluoro;
and in which R¹, R², R³, R⁴, L¹, L², n and m are as previously defined.

In one particular group of compounds of formula IB, R¹ represents H, methoxy, fluoro, chloro or dimethylamino; R² represents H, methyl, methoxy, dimethylamino or N,N-dimethylcarbamoyl, L² represents CH₂, A is CH₂, t is 0 or 1; R³ and R⁴ are each H, and R⁵ is 3-thienyl, 1-methylpyrrol-2-yl, 1-methylindol-3-yl, 2,4-dimethoxypyrimidin-5-yl, 2-(phenylsulfonyl)-1,3-thiazol-5-yl, 1-methyl-5-(trifluoromethyl)-1H-pyrazol-3-yl]-2-thienyl, 1-[(2-chloro-1,3-thiazol-5-yl)methyl]-1H-indol-3-yl}, 5-(2-thienyl)thien-2-yl, 5-pyridin-2-yl-2-thienyl, 1,2,3-thiadiazol-4-yl, 4-chloro-1-methyl-1H-pyrazol-3-yl and quinolin-2-yl.

In another particular group of compounds of formula IB, R¹ represents fluoro, chloro or dimethylamino; R² represents H, methyl, methoxy, dimethylamino or N,N-dimethylcarbamoyl, L² represents CH₂, A is CH₂, t is 0 or 1; R³ and R ⁴ are each H, and R⁵ is 3-thienyl, 1-methylpyrrol-2-yl, 1-methylindol-3-yl, 2,4-dimethoxypyrimidin-5-yl, 2-(phenylsulfonyl)-1,3-thiazol-5-yl, 1-methyl-5-(trifluoromethyl)-1H-pyrazol-3-yl]-2-thienyl, 1-[(2-chloro-1,3-thiazol-5-yl)methyl]-1H-indol-3-yl}, 5-(2-thienyl)thien-2-yl, 5-pyridin-2-yl-2-thienyl, 1,2,3-thiadiazol-4-yl, 4-chloro-1-methyl-1H-pyrazol-3-yl and quinolin-2-yl.

The term “pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt,or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. Compounds of formula I may exist as tautomers. AL¹ such tautomers and mixtures thereof are included in the scope of the present invention.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl . Preferred alkyl groups are methyl, ethyl, propyl, isopropyl and tertiary butyl.

Unless otherwise stated or indicated, the term “alkoxy” denotes a group O-alkyl, wherein alkyl is as defined above for example methoxy, ethoxy, propoxy, isopropoxy and butoxy.

Unless otherwise stated or indicated, the term “halo” shall mean fluorine, chlorine, bromine or iodine.

Unless otherwise stated or indicated, the term “aryl” in R⁵ means phenyl or naphthyl.

Examples of a C₁₋₄ alkoxy group optionally substituted by one or more fluoro, include trifluoromethoxy, difluoromethoxy, fluoromethoxy and 4,4,4-trifluorobutoxy.

Examples of a C₁₋₄ alkyl group optionally substituted by one or more fluoro include trifluoromethyl, difluoromethyl and fluoromethyl.

Examples of a group OSO₂C₁₋₄alkyl, wherein the alkyl group is optionally substituted with one or more fluorine atoms include methylsulfonyloxy, ethylsulfonyloxy, n-propylsulfonyl-oxy, n-butylsulfonyloxy, 4,4,4-trifluorobutyl-1-sulfonyloxy and 3,3,3-trifluoropropyl-1-sulfonyloxy.

Examples of a group NR^{a}R^{b} in which R^{a} and R^{b} independently represent H or a C₁₋₄alkyl group include methylamino, ethylamino, propylamino, isopropylamino, butylamino dimethylamino, diethylamino, N-ethyl-N-methylamino and diusopropylamino.

Examples of a group NR^{a}R^{b} in which R^{a} and R^{b} together with the nitrogen atom to which they are attached represent a saturated 3 to 7 membered heterocyclic ring optionally including an O include pyrrolidino, morpholino and piperidino.

Examples of a group CONR^{c}R^{d} in which R^{c} and R^{d} independently represent H or a C₁₋₄alkyl group include N-methylcarbamoyl, N-ethylcarbamoyl, N-propylcarbamoyl, N,N-dimethylcarbamoyl, N-ethyl-N-methylcarbamoyl and N,N-diethylcarbamoyl

Examples of a group CONR^{c}R^{d} in which R^{c} and R^{d} together with the nitrogen atom to which they are attached represent a saturated 3 to 7 membered heterocyclic ring include pyrrolidinocarbonyl and piperidinocarbonyl.

Specific compounds of the invention include one or more of the following:
N,N-dimethyl-2-[(3-{[(5-pyridin-2-yl-2-thienyl)methyl]amino} cyclohexyl)amino]-quinoline-4-carboxamide;
(1S,3S)-N-(6-chloro-4-methylquinolin-2-yl)-N′-[(1-methyl-1H-indol-3-yl)methyl]cyclohexane-1,3-diamine;
(1S,3S)-N-(6-fluoro-4-methylquinolin-2-yl)-N′-(3-thienylmethyl)cyclohexane-1,3-diamine;
(1R,3R)-N-(6-fluoro-4-methylquinolin-2-yl)-N′-(3-thienylmethyl)cyclohexane-1,3-diamine;
(1S,3S)-N-(6-fluoro-4-methoxyquinolin-2-yl)-N′-(3-thienylmethyl)cyclohexane-1,3-diamine;
(1S,3S)-N-(6-fluoro-4-methylquinolin-2-yl)-N′-[(1-methyl-1H-indol-3-yl)methyl]cyclopentane-1,3-diamine;
N-(6-chloroquinolin-2-yl)-N′-(3-thienylmethyl)cyclohexane-1,3-diamine;
N-(6-chloroquinolin-2-yl)-N′-[(1-methyl-1H-pyrrol-2-yl)methyl]cyclohexane-1,3-diamine;
N-(6-chloroquinolin-2-yl)-N′-(quinolin-3-ylmethyl)cyclohexane-1,3-diamine;
N⁶,N⁶-dimethyl-N²-{3-[(3-thienylmethyl)amino]cyclohexyl}quinoline-2,6-diamine;
(1S,3S)-N-[(4-chloro-1-methyl-1H-pyrazol-3-yl)methyl]-N′-(6-methoxy-4-methylquinolin-2-yl)cyclopentane-1,3-diamine;
(1S,3S)-N-(6-methoxy-4-methylquinolin-2-yl)-N′-(1,2,3-thiadiazol-4-ylmethyl)cyclopentane-1,3-diamine;
(1S,3S)-N-(6-methoxy-4-methylquinolin-2-yl)-N′-[(5-pyridin-2-yl-2-thienyl)methyl]cyclopentane-1,3-diamine;
(1S,3S)-N-({1-[(2-chloro-1,3-thiazol-5-yl)methyl]-1H-indol-3-yl}methyl)-N′-(6-methoxy-4-methylquinolin-2-yl)cyclopentane-1,3-diamine;
(1S,3S)-N-(6-methoxy-4-methylquinolin-2-yl)-N′-({5-[1-methyl-5-(trifluoromethyl)-1H-pyrazol-3-yl]-2-thienyl}methyl)cyclopentane-1,3-diamine;
(1S,3S)-N-(2,2′-bithien-5-ylmethyl)-N′-(6-methoxy-4-methylquinolin-2-yl)cyclopentane-1,3-diamine;
N⁴,N⁴-dimethyl-N²-{3-[(3-thienylmethyl)amino]cyclohexyl}quinoline-2,4-diamine;
N⁴,N⁴-dimethyl-N²-[3-({[2-(phenylsulfonyl)-1,3-thiazol-5-yl]methyl}amino)-cyclohexyl]quinoline-2,4-diamine;
N²-(3-{[(2,4-dimethoxypyrimidin-5-yl)methyl]amino}cyclohexyl)-N⁴,N⁴-dimethylquinoline-2,4-diamine;
3-(6-methoxy-4-methylquinolin-2-yl)-N-methyl-N-(3-thienylmethyl)-3-azabicyclo[3.2.1]octan-8-amine;
6-methoxy-4-methyl-N-[((1R,2S)-2-{[(1-methyl-1H-indol-3-yl)methyl]amino}cyclopentyl)methyl]quinolin-2-amine;
(1S,3S)-N-(6-fluoro-4-methylquinolin-2-yl)-N′-[(1-methyl-1H-pyrrolo[2,3-b]pyridin-3-yl)methyl]cyclopentane-1,3-diamine;
(1S,3S)-3-[({3-[(7-methoxy-4-methylquinolin-2-yl)amino]cyclopentyl}amino)methyl]-1-methyl-1H-indole-6-carbonitrile;
(1S,3S)-N-(6-fluoro-4-methylquinolin-2-yl)-N′-[(1-methyl-1H-indol-2-yl)methyl]cyclopentane-1,3-diamine;
(1S,3S)-N-(6-fluoro-4-methylquinolin-2-yl)-N′-({1-[3-(trifluoromethyl)pyridin-2-yl]-1H-indol-3-yl}methyl)cyclopentane-1,3-diamine;
(1S,3S)-N-(6-fluoro-4-methylquinolin-2-yl)-N′-[(1-methyl-1H-indazol-3-yl)methyl]cyclopentane-1,3-diamine;
(1S,3S)-N-(7-methoxy-4-methylquinolin-2-yl)-N′-({1-[4-(trifluoromethyl)phenyl]-1H-pyrrol-3-yl}methyl)cyclopentane-1,3-diamine;
3-[({(1S,3S)-3-[(7-methoxy-4-methylquinolin-2-yl)amino]cyclopentyl}amino)methyl]-1-methyl-1H-indole-5-carbonitrile;
(1S,3S)-N-{[5-difluormethoxy-1H-indol-3-yl]methyl}-N′-(7-methoxy-4-methylquinolin-2-yl)cyclopentane-1,3-diamine;
(1S,2S,4R,6S)-N-(6-methoxy-4-methylquinolin-2-yl)-N′-(3-thienylmethyl)bicyclo[2.2.1 ]heptane-2,6-diamine;
(1R,2S,4S,6S)-N-(6-methoxy-4-methylquinolin-2-yl)-N′-(3-thienylmethyl)bicyclo[2.2.1 ]heptane-2,6-diamine;
(1S,2S,4R,6S)-N-(7-methoxy-4-methylquinolin-2-yl)-N′-[(1-methyl-1H-indol-3-yl)methyl]bicyclo[2.2.1 ]heptane-2,6-diamine;
6-methoxy-4-methyl-N-[(1S,2R)-2-({[(1-methyl-1H-indol-3-yl)methyl]amino}methyl)cyclopentyl]quinolin-2-amine;
(1S,3S)-N-(7-methoxy-4-methylquinolin-2-yl)-N′-[(1-methyl-1H-pyrrolo[3,2-h]quinolin-3-yl)methyl]cyclopentane-1,3-diamine;
(1S,3S)-N-(6-fluoro-4-methylquinolin-2-yl)-N′-[(1-methyl-1H-pyrrolo[2,3-c]pyridin-3-yl)methyl]cyclopentane-1,3-diamine;
(1S,3S)-N-(7-methoxy-4-methylquinolin-2-yl)-N′-[(1-methyl-1H-pyrrolo[3,2-b]pyridin-3-yl)methyl]cyclopentane-1,3-diamine;
(1S,3S)-N-(6-fluoro-4-methylquinolin-2-yl)-N′-(imidazo[1,2-a]pyridin-3-ylmethyl)cyclopentane-1,3-diamine;
(1S,3S)-N-{[5-(benzyloxy)-1-methyl-1H-indol-3-yl]methyl}-N′-(7-methoxy-4-methylquinolin-2-yl)cyclopentane-1,3-diamine;
(1S,3S)-N-(7-Methoxy-4-methylquinolin-2-yl)-N′-[3-(trifluoromethoxy)benzyl]cyclohexane-1,3-diamine;
(1S,3S)-N-(2,1,3-Benzothiadiazol-4-ylmethyl)-N′-(7-methoxy-4-methylquinoin-2-yl)cyclohexane-1,3-diamine;
(1S,3S)-N-[(1,3-Dimethyl-1H-pyrazol-4-yl)methyl]-N′-(7-methoxy-4-methylquinolin-2-yl)cyclohexane-1,3-diamine; and
(1S,3S)-N-(2-Bromo-4-methoxybenzyl)-N′-(7-methoxy-4-methylquinotin-2-yl)cyclohexane-1,3-diamine;
and pharmaceutically acceptable salts thereof.
   Methods of Preparation

The compounds of the invention may be prepared as outlined below according to any of the following methods. However, the invention is not limited to these methods, the compounds may also be prepared as described for structurally related compounds in the prior art.

Compounds of formula I may be prepared by reacting a compound of formula II 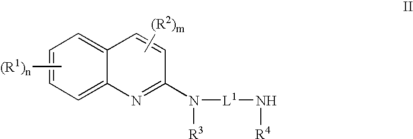
in which R¹, R², R³, R⁴, L¹, n and m are as previously defined with an aldehyde or a ketone of formula III 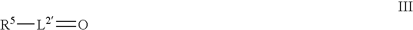
in which R⁵ is as previously defined and L² represents a group which after reaction of compounds II and III gives L² on reduction, under reductive alkylation conditions. For example, a compound of formula II and a compound of formula III may be reacted together at a temperature in the range of 0° C. to 250° C., preferably in the range of 50° C. to 150° C., optionally in the presence of an inert solvent, for example methanol, dichloromethane or acetic acid in the presence of a reducing agent, for example sodium cyanoborohydride or optionally polymer supported cyanoborohydride.

Compounds of formula II may be prepared by reacting a compound of formula IV 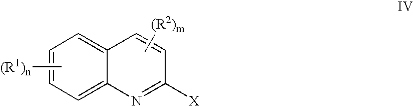
in which R¹, R², n and m are as previously defined and X is halo, particularly chloro or bromo, with a compound of formula V 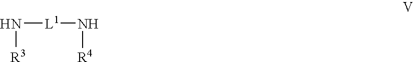
at a temperature in the range of 0° C. to 250° C., preferably in the range of 50° C. to 150° C. in pyridine or optionally in the presence of an inert solvent, for example toluene or dioxane in the presence of a catalytic cross-coupling system for example Pd(OAc)₂ and 2-(di-^{t}butylphosphino)biphenyl or BINAP, and optionally in the presence of a base for example NaO^{t}Bu or Cs₂CO₃.

Certain compounds of formula II and V are novel and are claimed as a further aspect of the present invention as useful intermediates.

Compounds of formula V, in which L¹ represents a bicyclic ring, for example: 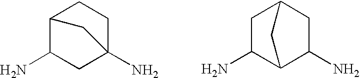
may be prepared e.g. starting from X (T., Poll; Tetrahedron Letters, 1989, 30,41, 5595-5598) or XI (G. L., Grunewald; J. Org. Chem. 1978, 43, 15, 3074-3076), utilizing standard techniques, e.g. Curtius rearangement and hydroboration, for conversion of carboxylic acids and olefins into amines.

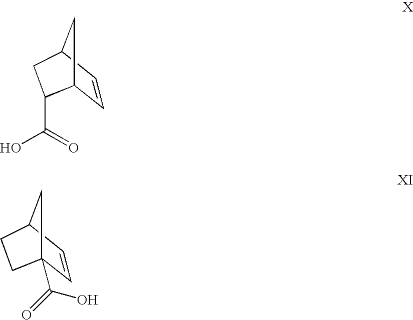

Compounds of formula V, in which L¹ represents a cyclopentylmethyl or tetrahydrofuryl-methyl, for example: 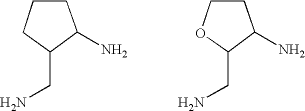
may be prepared e.g. as outlined in Bioorg. Med. Chem. Lett. 13, 1265-68 (2003), and references cited therein, or, alternatively, by conversion of compound XII into a diamine by standard transformations (e.g reduction of the acid to alcohol followed by conversion to amine via substitution of the corresponding sulfonate with azide followed by reduction).

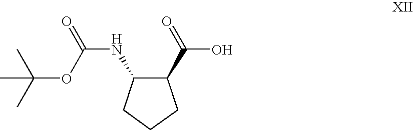

Optionally one or both nitrogens in formula V may be protected prior to reaction with a compound of formula IV and then the compound of formula II obtained is deprotected prior to reaction with a compound of formula III. Amine protecting groups are known to those skilled in the art for example the t-Boc, Cbz or phthalimido groups.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

The expression “inert solvent” refers to a solvent which does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free acid, or a pharmaceutically acceptable organic or inorganic base addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carrers.

The compounds of the invention may also be combined with other therapeutic agents which are useful in the treatment of disorders associated with obesity, psychiatric disorders, neurological disorders and pain.

Pharmacological Properties

The compounds of formula (I) are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. The compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents. The compounds may also eliminate the increase in weight that normally accompanies the cessation of smoking. The compounds are also potentially useful as agents for treating or preventing diarrhoea.

The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

Accordingly, it is desirable to provide a compound and method of treatment which will be active in reducing craving for the abused substance, and which does not exacerbate the sympathetic response rate caused by the abused substance and which has favorable pharmacodynamic effects.

The compounds are also potentially useful as agents for treating pain disorders, including but not limited to acute and chronic nociceptive, inflammatory and neuropathic pain and migraine.

In another aspect the present invention provides a compound of formula I as claimed in any previous claim for use as a medicament.

In a further aspect the present invention provides the use of a compound of formula I in the preparation of a medicament for the treatment or prophylaxis of obesity, psychiatric disorders such as psychotic disorders, anxiety, anxio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders , including but not limited to acute and chronic nociceptive, inflammatory and neuropathic pain and migraine, comprising administering a pharmacologically effective amount of a compound of formula I to a patient in need thereof.

In a still further aspect the present invention provides a method of treating obesity, psychiatric disorders such as psychotic disorders, anxiety, anxio-depressive disorders, depression, bipolar disorder, ADHD, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

The compounds of the present invention are particulary suitable for the treatment of obesity.

In another aspect the present invention provides a method of treating obesity, type II diabetes, Metabolic syndrome and a method of preventing type II diabetes comprising administering a pharmacologically effective amount of a compound of formula I to a patient in need thereof.

Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absortion, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt, solvate, solvate of such a salt or a prodrug thereof, may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

According to an additional further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-obesity compound for example orlistat (EP 129,748) and sibutramine (GB 2,184,122 and U.S. Pat. No. 4,929,629);
an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;
a CB1 antagonist or inverse agonist for example rimonabant;
another Melanin concentrating hormone (MCH) antagonist;
a PDK inhibitor; or
modulators of nuclear receptors for example LXR, FXR, RXR, and RORalpha;
an SSRI;
a serotonin antagonist;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for for the treatment of type 2 diabetes and its associated complications in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I, or a pharmaceutically acceptable salt, solvate, solvate of such a salt or a prodrug thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula I, or a pharmaceutically acceptable salt, solvate, solvate of such a salt or a prodrug thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### EXAMPLES

The invention will now be described in more detail with the following examples that are not to be construed as limiting the invention.

Abbreviations

aq. aqueous
Ac acetyl
BINAP rac-2,2′-Bis(diphenyl-phosphino)-1,1′-binaphtyl
Bu butyl
DCM dichloromethane
DMF N,N-dimethylformamide
ELS evaporative light scattering
Et ethyl
HEK human embryotic kidney
HPLC high performance liquid chromatography
LC liquid chromatography
MP-BH(OAc)₃ macroporous polymer bound triacetoxyborohydride (available from Argonaut)
MS mass spectroscopy
Pol-BH₃CN (polystyrylmethyl)trimethylammonium cyanoborohydride (loading 4.1-4.3 mmol BH₃CN/g)
Pol-CHO 4-benzyloxybenzaldehyde polystyrene (loading ˜2.66 mmol CHO/g)
tdd triplet of double doublets
TFA trifluoroacetic acid
THF tetrahydrofuran
TLC thin layer chromatography
Tris trishydroxymethylaminomethane
t tert
rt. room temperature
sat. saturated
br broad
bs broad singlet
bt broad triplet
d doublet
dd doublet of doublets
ddd double doublet of doublets
dt doublet of triplets
m multiplet
q quartet
s singlet
t triplet
tt triplet of triplets
td triplet of doublets
bd broad doublet
   General Experimental Procedures

Flash column chromatography employed MERCK normal phase silica gel 60 Å (40-63 μm), Isolute® pre-packed Flash Si columns, or a Biotage Horizon Pioneer® HPFC system equipped with FLASH 12+M or FLASH 25+M or 40+M silica cartridges. Mass spectra were recorded on a Waters Micromass ZQ single quadrupole equipped with a pneumatically assisted electrospray interface (LC-MS). Purifications were performed on a Waters Prep LC 2000 with UV-detection, equipped with a Kromasil 10 μm C8 250 mm×20 mm column, or on a semi preparative HPLC, Shimadzu LC-8A, Shimadzu SPD-10A UV-vis.-detector equipped with a Waters Symmetry® 100 mm×19 mm C18 5 μm column.

Automated HPLC purification was done using a Waters Fraction Lynx system equipped with UV, ELS and MS detection and an Ace C8 5μ 10 cm×21,2 id column. The mobile phase was A: 95% CH₃CN and B: 5% CH₃CN+95% 0,1 M NH₄OAc with a gradient from 100% B to 100% A in 10 minutes at 25 mL/min flow rate.

¹H NMR and ¹³C NMR spectra were obtained at 298 K on a Varian Unity Plus 400 mHz, or a Varian Inova 500 MHz or a Varian Unity Plus 600 MHz or a Bruker Avance 300 MHz. Chemical shifts are given in ppm with the solvent residual peak as internal standard: CDCl₃ δ_{H} 7.26, δ_{C} 77.2; MeOH-d₄ δ_{H} 3.31, δ_{C} 49.0; DMSO-d₆ δ_{H} 2.50; δ_{C} 39.5 ppm.

Microwave heating was performed using single node heating in a Smith Creator from Personal Chemistry, Uppsala, Sweden.

Analytical chiral HPLC was done using a Chiralcel OJ (250×4.6 mm i.d.) column with EtOH:Et₃N 100:0.1 as mobile phase at flow rate 1 mL/min and with UV detection at 254 or 350 nm.

Names/reference numbers of starting materials (CAS no), either commercially available or prepared by published methods.

cyclohexane-1,3-diamine, 3385-21-5; 2,4-dichloroquinoline, 703-61-7; (−)-2-azabicyclo[2.2.1]hept-5-en-3-one, 79200-56-9; 1-methylindole-3-carbaldehyde, 19012-03-4; 2-chloro-6-methoxy-4-methylquinoline. 6340-55-2; 4-fluoroaniline, 371-40-4; 3-thiophenecarbaldehyde, 498-62-4; 5381-20-4; rac-2,2′-bis(diphenylphosphino)-1,1′-binaphthyl (BINAP), 98327-87-8; 2-chloroquinoline-4-carboxylic acid, 5467-57-2; 2,6-dichloroquinoline, 151703-14-9; 2-chloro-6-fluoro-4-methylquinoline, 18529-12-9; 2,2′-bithiophene-5-carbaldehyde, 3779-27-9; 2,6-dichloro-4-methylquinoline, 90723-71-0; 1-methyl-5-(trifluoromethyl)-1H-pyrazol-3-yl]-2-thiophene-2-carbaldehyde, 175202-93-4; (2-chloro-1,3-thiazol-5-yl)methyl-1H-indole-3-carbaldehyde, 439095-43-9; 1,2,3-thiadiazol-4-carbaldehyde, 27643-15-8; 4-chloro-1-methyl-1H-pyrazole-3-carbaldehyde, 175204-81-6; quinoline-3-carbaldehyde, 13669-42-6; 1-methylpyrrole-2-carbaldehyde, 406695-47-4; 5-pyridin-2-ylthiophene-2-carbaldehyde, 132706-12-8; 2-(phenylsulfonyl)-1,3-thiazole-5-carbaldehyde, 477886-95-6; 2,4-dimethoxypyrimidine-5-carbaldehyde, 52606-02-7; 5-pyridine-2-yl-thiophene-2-carbaldehyde 13270-12-8.

2-bromopropane, 75-26-3; chlorodifluoromethane, 75-45-6; ethyl acetoacetate, 141-97-9; 3-fluoroaniline, 372-19-0; o-anisidine, 90-04-0; 2-chloro-7-methoxy-4-methylquinoline, 97892-67-6; m-anisidine, 536-90-3; 1-methyl-1H-indazole-3-carboxylic acid, 186129-25-9; 1H-pyrrolo[2,3-b]pyridine, 271-63-5; 1-methyl-1H-indole-2-carbaldehyde, 19012-03-4; 4-aminobenzotrifluoride, 455-14-1; 2,5-dimethoxy-3-tetrahydrofurancarboxaldehyde, 50634-05-4; 6-chloro-5-fluoro-1H-indole, 122509-72-2; 1H-indole-5-carbonitrile, 15861-24-2; 1H-indole-6-carbonitrile, 15861-36-6; 1H-Indol-5-ol, 1953-54-4; 5-fluoro-1H-indole-3-carbaldehyde, 2338-71-8; 5-chloro-1H-indole-3-carbaldehyde, 827-01-0; 5-bromo-1H-indole-3-carbaldehyde, 877-03-2; dimethylcarbamyl chloride, 79-44-7; D(+)-malic acid, 97-67-6; cyclopentadien, 542-92-7; (1S,2S)-2-[(tert-butoxycarbonyl)amino]cyclopentanecarboxylic acid, 143679-80-5; 1H-pyrrolo[2,3-c]pyridine, 271-29-4; imidazo[1,2-a]pyridine, 274-76-0; (benzyloxy)-1H-indole, 1215-59-4.

2,1,3-benzothiadiazole-4-carbaldehyde, 5170-68-3; 3-(trifluoromethoxy)benzaldehyde, 52771-21-8; 2-bromo-5-methoxybenzaldehyde, 7507-86-0; 1,3-dimethyl-1H-pyrazole-4-carbaldehyde, 25016-12-0; 3,4-dichlorobenzaldehyde, 6287-38-3.

Preparation of Intermediates

### Tert-butyl [(1S,3S)-3-aminocyclopentyl]carbamate

### a) (1R,3S)-3-[(tert-butoxycarbonyl)amino]cyclopentyl methanesulfonate

Prepared according to WO9811103 from (−)-2-azabicyclo[2.2.1]hept-5-en-3-one (>95% ee).

### b) Tert-butyl [(1S,3S)-3-azidocyclopentyl]carbamate

NaN₃ (16.6 g, 0.25 mmol) was added to a stirred solution of (1R,3S)-3-[(tert-butoxycarbonyl)amino]cyclopentyl methanesulfonate (20 g, crude, ˜0.05 mol) in DMF (250 mL) under nitrogen atmosphere. The mixture was heated to 50° C. for 18 h (over night). The mixture was allowed to reach rt., poured into H₂O (200 mL), extracted with EtOAc (2×400 mL), 200 mL Et₂O and concentrated. Purification of the residue by flash chromatography [280 g silica gel, 6×22 cm column, with EtOAc/heptane (2:3→1:1) as eluent]afforded the title compound (16.5 g, contaminated with DMF) as a slightly yellowish oil taken to the next step without further purification.

¹H NMR (300.1 MHz, CDCl₃) δ 4.52 (bs, 1H), 4.00-4.10 (m, 2H), 1.98-2.22 (m, 3H), 1.62-1.78 (m, 2H), 1.42-1.52 (m, 1H), 1.44 (s, 9 H).

### c) Tert-butyl [(1S,3S)-3-aminocyclopentyl]carbamate

A flask containing tert-butyl [(1S,3S)-3-aminocyclopentyl]carbamate (16.5 g, crude ˜0.05 mol) and 1.7 g Pd-C (10% paste) in MeOH (300 mL) was exposed to a positive pressure of hydrogen gas (balloon) over weekend. The catalyst was filtered off and the mixture was concentrated to afford the title compound (9.5 g) as a thick colorless viscous oil.

¹H NMR (300.1 MHz, DMSO-d₆) δ 6,74 (bd, 1H), 3.86-3.92 (m, 1H), 3.28 (quintet, 1H), 1.73-1.98 (m, 2H), 1.43-1.59 (m, 2H), 1.22-1.41 (m, 1H), 1.36 (s, 9 H), 1.07-1.20 (m, 1H). ¹³C NMR (DMSO-d₆) δ 155.0, 77.2, 50.8, 50.0, 42.6, 34.2, 31.2, 28.3. LC-MS [M+H]⁺ 201

### (1S,3S)-N-(6-methoxy-4-methylquinolin-2-yl)cyclopentane-1,3-diamine

### a) Tert-butyl {(1S,3S)-3-[(6-methoxy-4-methylquinolin-2-yl)amino]cyclopentyl}carbamate

A mixture of 2-chloro-6-methoxy-4-methylquinoline (0.690 g, 3.33 mmol), tert-butyl [(1S,3S)-3-aminocyclopentyl]carbamate (1.00 g, 5.0 mmol), NaO^{t}Bu (4.66 mmol, 0.45 g), Pd(OAc)₂ (0.075 g, 0.33 mmol), and BINAP (0.207 g, 0.33 mmol) in toluene (30 mL) was stirred at 100° C. under nitrogen until LC-MS indicated that starting material was consumed. The reaction mixture was cooled to room temperature, poured into Et₂O (300 mL) and washed with brine. The organic layer was then separated, dried over Na₂SO₄ and evaporated to dryness. The residue was purified on a SiO₂ column eluted with DCM:MeOH (95:5) to give 0.618 g (50%) of the title compound.

### b) (1S,3S)-N-(6-methoxy-4-methylquinolin-2-yl)cyclopentane-1,3-diamine

Tert-butyl {(1S,3S)-3-[(6-methoxy-4-methylquinolin-2-yl)amino]cyclopentyl}carbamate (0.550 g, 1.48 mmol) and TFA (3 mL) in CHCl₃ (7 mL) was stirred at rt. for 6 hours. LC indicated that starting material was consumed. The mixture was then evaporated to dryness. pH was set to 10 with a 2 N NaOH solution and then extracted with EtOAc. The organic layer was separated, dried on MgSO₄ and concentrated, to give 0.400 g (99%) of the title compound.

¹H NMR (300.1 MHz, CDCl₃) δ 7.57 (d, 1H), 7.16-7.20 (dd 1H), 7.04 (d, 1H), 6.51 (s, 1H), 5.24 (br, 1H), 4.44 (m, 1H), 3.86 (s, 3H), 3.50 (m, 1H), 2.73 (br, 2H), 2.51 (s, 3H), 2.26 (m, 2H), 2.06 (m, 1H), 1.85 (m, 1H), 1.41 (m, 2H). LC-MS [M+H]⁺ 272

### 2-Chloro-N,N-dimethylquinolin-4-amine

Prepared from 2,4-dichloroquinoline according to literature procedure: T. Watanabe, et al; Synthesis 1980, pp 39-41.

¹H NMR (300.1 MHz, DMSO-d₆) δ8.01 (d, 1H), 7.98 (d, 1H), 7.62 (dd, 1H), 7.43 (dd, 1H), 6.70 (s, 1H), 3.05 (s, 6H). LC-MS [M+H]⁺ 207

### 2-Chloro-N,N-dimethylquinolin-6-amine

### a) 1-Methyl-6-nitroquinolin-2(1H)-one

Prepared by a modification of the procedure described by H. von Balli and D. Schelz, Helv. Chim. Acta, Vol. 53 (1970) pp 1903-1912, using 15 M HNO₃ and rt. as reaction temperature instead of what is written. ¹H NMR (300.1 MHz, DMSO-d₆) δ in agreement with those described by: N. Nishiwaki et al. Tetrahedron, Vol. 58 (2002) pp 473-478.

### b) 2-Chloro-6-nitroquinoline

Prepared according to the procedure described by H. von Balli and D. Schelz, Helv. Chim. Acta, Vol. 53 (1970) pp 1903-1912.

### c) 2-Chloroquinoline-6-amine

SnCl₂.2 H₂O (42 g, 0.19 mol) was added to a stirred solution of 2-chloro-6-nitroquinoline (8.1 g, 39 mmol) in EtOH (250 mL). The mixture was refluxed for 0.5 h, cooled to rt., concentrated and dissolved in DCM (200 mL), added NaOH (150 mL, aq., 5 M) filtered and rinsed with H₂O (150 mL) followed by Et₂O (100 mL). The organic phase was washed with NaHCO₃ (100 mL, aq., sat.) and concentrated, which afforded the title compound (4.9 g, 70%) as an orange-yellow, solid material, used in next step without further purification.

¹H NMR (300.1 MHz, DMSO-d₆) δ 8.01 (d, 1H), 7.62 (d, 1H), 7.30 (d, 1H), 7.19 (dd, 1H), 6.83 (d, 1H), 5.73 (s, 2H). LC-MS [M+H]⁺ 179

### d) 2-Chloro-N,N-dimethylquinolin-6-amine

MeI (2.8 g, 20 mmol) was added to a stirred solution of 2-chloroquinoline-6-amine (4.7 g, 25 mmol) and K₂CO₃ (3.6 g, 26 mmol) in DMF (300 mL) under nitrogen atmosphere. The mixture was heated to 70° C. for 0.5 h and additional MeI (0.9 g, 6 mmol) was then added, and then stirred for 5 h. The mixture was allowed to reach rt. and poured into H₂O (200 mL) and extracted with DCM (2×200 mL) and concentrated. Purification of the residue by flash chromatography [120 g silica gel, 6×9 cm column, with EtOAc/heptane (2:3→3:2) followed by DCM:MeOH (95:5+1% Et₃N) as eluent] afforded a mixture of mono- and di-N-methylated compounds (0.9 g) as an yellow solid material. The unreacted 2-chloroquinoline-6-amine isolated (2.8 g) was reacted again in the same manner as described above (1.7 g+0.7 g MeI, 2.3 g K₂CO₃, 175 mL DMF) to give an additional 1.7 g of product mixture. The is combined batches were purified by flash chromatography (SiO₂, Heptane:EtOAc) to yield 0.91 g of the title compound.

¹H NMR (300.1 MHz, DMSO-d₆) δ 8.15 (d, 1H), 7.75 (d, 1H), 7.48 (dd, 1 H), 7.38 (d, 1H), 6.99 (d, 1H), 3.04 (s, 3H), 3.02 (s, 3H). LC-MS [M+H]⁺ 207

### Dibenzyl trans-cyclohexane-1,3-diylbiscarbamate

D-tartaric acid (15.77 g, 105 mmol) was added to a stirred solution of cyclohexane-1,3-diamine (12 g, 105 mmol, cis/trans ˜2.6:1) in H₂O (80 mL). The resulting mixture was heated to ˜60° C. and MeOH (800 mL) was slowly added. The mixture was allowed to attain rt and left for 3 days. The precipitate was filtered off and the filtrate was concentrated and redissolved in 1M NaOH (40 mL). To the stirred mixture at 0° C. was added benzyl chloroformate (9.56 g, 56 mmol) and 1M NaOH (40 mL). After 5 min, 1,4-dioxane (40 mL) was added and the mixture stirred for an additional 18 h at rt. The mixture was diluted with H₂O and extracted with CH₂Cl₂. The organic layer was dried with MgSO₄, filtered and concentrated. Purification on a Biotage Horizon 40+M SiO₂ column gave 5.61 g (14%) of the title compound as a white solid.

¹H NMR (400 MHz, MeOH-d₄) δ 7.36-7.26 (m, 5H), 5.06 (bs, 2H), 3.77 (b, 2H), 1.73-1.42 (m, 8H). LC-MS [M+H]⁺ 383.4

### (+) Dibenzyl-trans-cyclohexane-1,3-diylbiscarbamate

The enantiomers of dibenzyl-trans-cyclohexane-1,3-diylbiscarbamate were separated by preparative chiral chromatography. 7.27 g were dissolved in EtOH (56 mg/mL), repeated 2 mL (112 mg) injections on a Chiralcel OJ (250×20 mm i.d.), eluted with EtOH:Et₃N 100/0.1, 12 mL/min, gave 3.75 g of the title compound, 99.3% ee, [α]²⁰_{D}+2.7 (c 1.26, MeOH) and 2.45 g of (−)dibenzyl-trans-cyclohexane-1,3-diylbiscarbamate, 83% ee.

### (1S,3S)-Cyclohexane-1,3-diamine dihydrochloride

(+)dibenzyl-trans-cyclohexane-1,3-diylbiscarbamate (0.24 mmol, 0.090 g) and 10% Pd on activated carbon (0.010 g) in EtOH (5 mL) was stirred under a H₂-atmosphere. After 1 h, the mixture was filtered through Celite and concentrated to give 44 mg of the title compound (100%). The product was recrystallized from MeOH/Et₂O and the absolute configuration was determined by X-ray crystallography.

### 2-Chloro-6-fluoro-4-methoxyquinoline

### a) 2,4-Dichloro-6-fluoro-quinoline

To a mixture of 4-fluoroaniline (8.5 g, 76.5 mmol) and malonic acid (8.0 g, 76.9 mmol) was added POCl₃ (160 g, 1.04 mol) and the mixture was slowly heated to 100° C. and then kept at this temperature for 18 h. The reaction mixture was cooled to room temperature and poured into ice-water (1.0 L). The brown slurry was filtered and the solid brown/orange material was purified by flash chromatography [350 g SiO₂, 6×24 cm column, eluting with DCM], which afforded 3.37 g (20%) of the title compound as an off-white solid.

### b) 2-Chloro-6-fluoro-4-methoxy-quinoline

To 2,4-dichloro-6-fluoro-quinoline (3.3 g, 15 mmol) in MeOH (50 mL) was added NaOMe (2.5 g, 46 mmol) at rt. under an atmosphere of nitrogen. The slurry was heated at reflux for 2 h, cooled to rt. and concentrated. The residue was purified by flash chromatography [60 g SiO₂, 4×12 cm column, eluting with DCM], which afforded 2.17 g (69%) of the title compound as a white solid material.

¹H NMR (300.1 MHz, CDCl₃) δ 7.89 (dd, 1H), 7.68 (dd, 1H), 7.43 (ddd, 1H), 6.71 (s, 1H), 4.02 (s, 3H). LC-MS [M+H]⁺ 212

### 2-Chloro-7-methoxy-4-methylquinoline

### a) N-(3-Methoxy-phenyl)-3-oxo-butyramide

Prepared by the procedure described for preparation of N-aryl-3-oxobutanamides in: Frohberg, P.; Drutkowski, H.; Wagner, C. Eur. J. Org. Chem. 2002, 1654-1663, with m-anisidine as aryl component.

¹H NMR (CDCl₃) δ 9.07 (br s, 1H), 7.17-7.30 (m, 2H), 7.03 (d, 1H), 6.67 (dd, 1H), 3.80 (s, 3H), 2.58 (s, 2H), 2.33 (s, 3H). MS (ESI) 208.2 (M+H⁺).

### b) 2-Hydroxy-7-methoxy-4-methylquinoline

N-(3-Methoxy-phenyl)-3-oxo-butyramide (103 g, 0.497 mol) was added in portions to 110 mL sulfuric acid (conc.) at 0° C. The mixture was heated to 100° C. and then kept at this temperature for 1.5 h. The reaction mixture was cooled to rt. and poured into ice-water (400 mL). The solid product thus obtained was filtered and then suspended in water (200 mL) and neutralized with 145 mL NH₂OH (25% aq.). The crude product was filtered off and suspended in CH₂Cl₂:EtOH (3:1, 300 mL). The suspension was filtered and the filtrate was evaporated to give the title compound as a solid. This was recrystallized twice in EtOH to give 27 g (29%) the title compound as a white solid. ¹H NMR (CDCl₃) δ 12.5 (br s, 1H), 7.56 (d, 1H), 6.87 (d, 1H), 6.82 (dd, 1H), 6.44 (s, 1H), 3.90 (s, 3H), 2.46 (s, 3H). MS (ESI) 190.1 (M+H⁺).

### c) 2-Chloro-7-methoxy-4-methylquinoline

2-Hydroxy-7-methoxy-4-methylquinoline (27.3 g, 144 mmol) was added to POCl₃ (220 g, 1.44 mol) at 0° C. followed by heating to 110° C. for 0.5 h. The mixture was cooled to room temperature, poured into ice-water (1.2 L) and stirred over night. Extraction with dichloromethane and concentration gave a white solid. Recrystallization from EtOH and a few drops of water gave 12.3 g (41%) of the title compound as white needles. ¹H NMR (CDCl₃) δ 7.82 (d, 1H), 7.36 (d, 1H), 7.20 (dd, 1H), 7.11 (s, 1H), 3.93 (s, 3H), 2.64 (s, 3H). ¹³C NMR (CDCl₃) δ 161.1, 150.8, 149.4, 147.4, 124.8, 121.8, 120.2, 119.2, 107.3, 55.5, 18.5. MS (ESI) 208.1 (M+H⁺).

### 2-Chloro-7-fluoro-4-methylquinoline

### a) N-(3-Fluoro-phenyl)-3-oxo-butyramide

Prepared by the procedure described for preparation of N-aryl-3-oxobutanamides in: Frohberg, P.; Drutkowski, H.; Wagner, C. Eur. J. Org. Chem. 2002, 1654-1663, with 3-fluoroaniline as aryl component. ¹H NMR (CDCl₃) δ 9.26 (br s, 1H), 7.51 (m, 1H), 7.14-7.32 (m, 2H), 6.81 (ddd, 1H), 3.59 (s, 2H), 2.33 (s, 3H). MS (ESI) 196.1 (M+H⁺).

### b) 7-Fluoro-4-methylquinolin-2-ol

N-(3-Fluoro-phenyl)-3-oxo-butyramide (11.2 g, 57 mmol) was added in portions to 10 mL sulfuric acid. The mixture was heated to 95° C. and then kept at this temperature for 15 min. The reaction mixture was cooled to rt. and poured into ice-water (40 mL). The resulting slurry was suspended in water (200 mL) and neutralized with approx. 20 mL NH₄OH (25% aq.). The crude product was filtered off and suspended in CH₂Cl₂:EtOH (1:1, 250 mL). The suspension was filtered and the filtrate was concentrated to approx. ⅔ of its volume. Recrystallization of this filtrate gave 2.1 g (22%) of the title compound as a white solid. ¹H NMR (CDCl₃) δ 12.3 (br s, 1H), 7.66 (dd, 1H), 7.13 (dd, 1H), 6.97 (ddd, 1H), 6.54 (s, 1H), 2.50 (s, 3H). MS (ESI) 178.1 (M+H⁺).

### c) 2-Chloro-7-fluoro-4-methylquinoline

7-Fluoro-4-methylquinolin-2-ol (2.1 g, 12 mmol) was added to POCl₃ (25 g, 160 mol) at rt. followed by heating to reflux. After 10 min at this temperature the mixture was cooled to rt., poured into ice-water (150 mL) and stirred at ambient temperature over night. Extraction with dichloromethane and concentration of the organic phases gave a white solid. Recrystallization from EtOH and a few drops of water gave 1.4 g (60%) of the title compound as a white solid. ¹H NMR (CDCl₃) δ 7.95 (dd, 1H), 7.63 (dd, 1H), 7.34 (ddd, 1H), 7.20 (s, 1H), 2.67 (s, 3H). ¹³C NMR (CDCl₃) δ 163.6 (d, J=251 Hz), 152.0, 149.0 (d, J=13 Hz), 147.9, 126.1 (d, J=10 Hz), 124.2, 122.1 (d, J=2 Hz), 117.0 (d, J=25 Hz), 113.2 (d, J=21 Hz), 18.8. MS (ESI) 196.1 (M+H⁺).

### 2-Chloro-7-difluoromethoxy-4-methylquinoline

A mixture of 2-chloro-4-methylquinolin-7-ol and 2-bromo-4-methylquinolin-7-ol (2.12 g, approx. 10 mmol) and KOH (1.6 g, 30 mmol) was dissolved in 2-propanol. Chlorodifluoromethane (Freon 22) was bubbled into the reaction, with vigorous stirring, in 5 to 30 min periods during 2 h (temperature was kept below 40° C.). The reaction mixture was poured into H₂O (75 mL) and extracted with CH₂Cl₂ (100 and 50 mL). The combined organic phases was washed with NaOH (1.5 M, aq.) and concentrated. Filtration of the residue through silica gel, eluting with MeOH, followed by concentration and recrystallization of the residue in EtOH gave the title compound as a ˜2:1 mixture with 2-bromo-7-difluoromethoxy-4-methyl-quinoline, in total 1.83 g (approx. 70% yield).

¹H NMR (CDCl₃) δ 7.97 (d, 1H), 7.68 (m, 1H), 7.36 (m, 1H), 7.23 (s, 1H), 6.68 (t, 1H, OCHF₂), 2.68 (s, 3H). MS (ESI) 244.1 (M+H⁺).

### 2-Chloro-7-isopropoxy-4-methylquinoline

### a) 2-Chloro-4-methyl-quinolin-7-ol

2-Chloro-7-methoxy-4-methylquinoline (12.1 g, 58 mmol) was stirred in HBr (130 mL, 48% aq.) at reflux temperature for 2 days and then cooled on an ice-bath. To the mixture was added H₂O (40 mL) and the mixture was then cautiously made basic with NaOH (270 mL, 5 M, aq.) and filtered. The filtrate was neutralized with HCl (50 mL, 10% aq.) and AcOH (10 mL). The is solid material was filtered, recrystallized from MeOH and dried to give the title compound as a ˜2:1 mixture with 2-bromo-4-methyl-quinolin-7-ol, in total 8.7 g (approx. 70-80% yield).

¹H NMR (DMSO-d₆) δ 7.94 (d, 1H), 7.15-7.24 (m, 2H), 7.14 (s, 1H), 2.61 (s, 3H). MS (ESI) 194.1 (M+H⁺).

### b) 2-Chloro-7-isopropoxy-4-methylquinoline

The mixture of 2-chloro-4-methylquinolin-7-ol and 2-bromo-4-methylquinolin-7-ol (2.0 g, approx. 10 mmol, from above) was heated to 80° C. with 2-bromopropane (1.95 mL, 21 mmol) and Cs₂CO₃ (5.0 g, 15 mmol) in DMF (35 mL) for 15 h. The reaction mixture was cooled to rt., poured into H₂O (50 mL), extracted with CH₂Cl₂ (2×75 mL) and concentrated. Purification of the residue by flash chromatography [40 g silica gel, 4×7 cm column, eluting with EtOAc/heptane (1:4)] afforded the title compound as a ˜2:1 mixture with 2-bromo-7-isopropoxy-4-methyl-quinoline, in total 2.1 g (approx. 80% yield) as an oil. The oil was made to solidify by adding a few drops of Et₂O followed by evaporation under vacuum.

¹H NMR (CDCl₃) δ 7.82 (d, 1H), 7.34 (m, 1H), 7.17 (m, 1H), 7.08 (s, 1H), 4.71 (m, 1H), 2.63 (s, 3H), 1.41 (d, 6H). MS (ESI) 236.2 (M+H⁺).

### 2-Chloro-7-methylsulfonyloxy-4-methylquinoline

To a mixture of 2-chloro-4-methylquinolin-7-ol and 2-bromo-4-methylquinolin-7-ol (1.85 g, approx. 9 mmol) in CH₂Cl₂ (50 mL) was added triethylamine (1.0 g, 10 mmol) and methanesulfonyl chloride (1.1 g, 9.5 mmol). The mixture was allowed to reach rt. and was kept at this temperature for 2 h. The reaction mixture was poured into NaHCO₃ (50 mL, aq., sat.) and the phases were separated. The aqueous phase was extracted with CH₂Cl₂ and the combined organic phases were concentrated. Purification of the residue by flash chromatography [30 g silica gel, eluting with CH₂Cl₂/MeOH (98:2)] afforded the title compound as a ˜2:1 mixture with 2-bromo-7-methylsulfonyloxy-4-methylquinoline, in total 2.16 g (approx. 85% yield) as a white solid.

¹H NMR (CDCl₃) δ 8.04 (d, 1H), 7.91 (d, 1H), 7.57 (dd, 1H), 7.30 (s, 1H), 3.23 (s, 3H), 2.71 (s, 3H). MS (ESI) 272.1 (M+H⁺).

### 2-Chloro-8-methoxy4-methylquinoline

### a) N-(2-Methoxy-phenyl)-3-oxo-butyramide

O-anisidine (30 g, 0.24 mol) was added to ethyl acetoacetate (160 g, 1.22 mol) at 100° C. and the mixture was heated at reflux at 160° C. under an atmosphere of nitrogen. After 19 h the mixture was allowed to cool and ethyl acetate (500 mL) was added to the mixture and the solution was washed twice with a 10% aq. HCl solution (150 mL). The organic layer was dried over Na₂SO₄ and evaporated. The resulting yellow residue was recrystallized from Et₂O, giving 10.53 g (50.7 mmol) of the title compound.

¹H NMR (CDCl₃) δ 8.02 (d, 1H), 7.63 (dd, 1H), 6.90-7.09 (m, 3H), 3.88 (s, 3H), 3.66 (s, 3H), 2.26 (s, 3H). MS (ESI) 208.1 (M+H⁺).

### b) 8-Methoxy-4-methyl-1H-quinolin-2-one

N-(2-Methoxy-phenyl)-3-oxo-butyramide (24.4 g, 0.12 mol) was added portion wise to sulfuric acid (30.5 g, 0.31 mol) at 0° C. The mixture was stirred at 95° C. under an atmosphere of nitrogen. The mixture was cooled to room temperature after which it solidified. Ice-cold water was added to the solid and the aq. solution was made basic with 25% aq. ammonia and extracted with ethyl acetate. The organic layer was separated and dried over Na₂SO₄. Purification of the residue by flash chromatography [eluting with CH₂Cl₂/MeOH (95:5)] gave 6 g (31.7 mmol) of the title compound as a yellow solid. ¹H NMR (DMSO-d₆) δ 10.58 (br s, 1H), 7.26-7.29 (m, 1H), 7.13-7.16 (m, 2H), 6.42 (d, 1H), 3-89 (s, 3H), 2.41 (s, 3H). MS (ESI) 190.1 (M+H⁺).

### c) 2-Chloro-8-methoxy-4-methylquinoline

8-Methoxy-4-methyl-1H-quinolin-2-one (6 g, 31.7 mmol) was dissolved in POCl₃ (30 mL) and the mixture was stirred at 110° C. under an atmosphere of nitrogen. The mixture was cooled to room temperature, poured over ice and extracted with CH₂Cl₂. The organic layer was separated and dried over Na₂SO₄. After evaporation of the solvent the residue was purified by flash chromatography (eluting with CH₂Cl₂) to give 4.73 g of the title compound as a solid.

¹H NMR (CDCl₃) δ 7.42-7.47 (m, 2H), 7.23 (s, 1H), 7.01-7.10 (m, 1H), 4.03 (s, 3H), 2.62 (s, 3H). ¹³C NMR (CDCl₃) δ 155.1, 149.7, 147.7, 139.5, 128.2, 126.9, 123.3, 115.5, 108.7, 56.1, 19.1. MS (ESI) 208.1 (M+H⁺).

### (1S,3S)-N-(7-Methoxy-4-methylquinolin-2-yl)cyclopentane-1,3-diamine

### a) (1S,3S)-tert-Butyl {3-[(7-methoxy-4-methylquinolin-2-yl)amino]cyclopentyl}carbamate

2-Chloro-7-methoxy-4-methylquinoline (0.455 g, 2.19 mmol), (1S,3S)- tert-butyl (3-aminocyclopentyl)carbamate (0.605 g, 3.02 mmol), palladium acetate (54 mg, 0.24 mmol), BINAP (0.151 g, 0.243 mmol) and cesium carbonate (1.97 g, 6.04 mmol) were added to 7 mL of dioxan in a microwave tube equipped with a magnetic stirring bar. The tube was capped and flushed with argon and the mixture was stirred and heated at 70° C. for 4 h. The mixture was filtered through celite which was washed with dioxan. The filtrate was evaporated and the residue was partitioned between water and EtOAc. The aqueous layer was extracted with EtOAc and the combined organic layer was washed with water, dried over Na₂SO₄, filtered and evaporated. The crude product was purified on a 300×50 mm Kromasil C8 column 100 Å 10μ and eluted with a gradient of CH₃CN:0.1M NH₄OAc 10:90-100:0. The pertinent fractions were combined and the organic solvent evaporated. The residue was made alkaline by NaOH (aq) and extracted three times with EtOAc. The combined organic layer was washed with water, dried over Na₂SO₄, filtered and evaporated. Yield: 0.422 g (52%).

1H NMR (500 MHz, CDCl₃) δ 7.63 (d, 1H), 7.05 (d, 1H), 6.87 (dd, 1H), 6.32 (s, 1H), 4.65-4.50 (m, 2H), 4.43 (m, 1H), 4.13 (m, 1H), 3.90 (s, 3H), 2.51 (s, 3H), 2.31 (m, 1H), 2.21 (m, 1H), 2.00 (m, 1H), 1.95 (m, 1H), 1.60-1.45 (m, 11H, thereof 1.45 (s, 9H)).

### b) (1S,3S)-N-(7-Methoxy-4-methylquinolin-2-yl)cyclopentane-1,3-diamine

(1S,3S)-tert-Butyl {3-[(7-methoxy-4-methylquinolin-2-yl)amino]cyclopentyl}carbamate (0.327 g, 0.880 mmol; from step a above) was dissolved in 12 mL of DCM and 3 mL of TFA was added. The mixture was allowed to react for 30 min and then evaporated. The residue was poured into water which was made alkaline by NaOH (aq) and extracted three times with EtOAc. The combined organic layer was washed with water, dried over Na₂SO₄, filtered and evaporated. Yield: 0.23 g (96%).

¹H NMR (500 MHz, CDCl₃) δ 7.63 (d, 1H), 7.03 (d, 1H), 6.85 (dd, 1H), 6.37 (s, 1H), 4.81 (bs, 1H), 4.41 (m, 1H), 3.89 (s, 3H), 3.56 (m, 1H), 2.51 (s, 3H), 2.34 (m, 1H), 2.08 (m, 1H), 1.90-1.80 (m, 2H), 1.72 (bs, 2H), 1.51 (m, 1H), 1.40 (m, 1H).

### 1-Methyl-1H-indazole-3-carbaldehyde

### a) (1-Methyl-1H-indazol-3-yl)methanol

1-Methyl-1H-indazole-3-carboxylic acid (0.500 g, 2.84 mmol) was dissolved in dry THF and Et₃N (0.435 mL, 3.12 mmol) was added. The mixture was stirred and cooled to −18° C. and isobutyl chloroformate (0.426 g, 3.12 mmol) was added dropwise. After 30 min the slurry was filtered and the filtrate was cooled again to −18° C. Sodium borohydride (0.322 g, 8.51 mmol) was added plus a few drops of water. When foaming had subsided 8 mL of water was added, the cooling bath was removed and the reaction mixture was stirred for 1 h. The THF was evaporated and the residue was diluted with a few mL of water and extracted three times with EtOAc. The combined organic layer was washed with water, dried over Na₂SO₄, filtered and evaporated. The crude product was chromatographed on a pre-packed SiO₂-column (Isolute, 20 g) eluted with DCM:MeOH 95:5. Yield: 0.320 g (70%).

¹H NMR (500 MHz, CDCl₃) δ 7.80 (d, 1H), 7.39 (m, 1H), 7.32 (d, 1H), 7.15 (t, 1H), 5.01 (bd, 2H), 3.96 (s, 3H), 2.82 (bs, 1H).

### b) 1-Methyl-1H-indazole-3-carbaldehyde

(1-Methyl-1H-indazol-3-yl)methanol (0.320 g, 1.97 mmol, from step a above) was dissolved in 25 mL of DCM and Dess-Martin periodinane (0.920 g, 2.17 mmol) was added. The mixture was stirred for 30 min after which 150 mL of diethyl ether was added and the suspension was hydrolysed by addition of 50 ml of 2M NaOH and stirring for 10 min. The ether layer was washed with 1M NaOH and water, dried over Na₂SO₄, filtered and evaporated. The crude product was chromatographed on a pre-packed SiO₂-column (Isolute, 10 g) eluted with DCM:MeOH 98:2. Yield: 0.271 g (86%).

¹H NMR (300 MHz, CDCl₃) δ 10.21 (s, 1H), 8.29 (m, 1H), 7.50-7.43 (m, 2H), 7.36 (m, 1H), 4.18 (s, 3H).

### 1-[4-(trifluoromethyl)phenyl]-1H-pyrrole-3-carbaldehyde

To a solution of 2,5-dimethoxy-3-tetrahydrofurancarboxaldehyde (8.0 g, 49.9 mmol) in acetic acid (120 mL) was added 4-aminobenzotrifluoride (8.05 g, 49.9 mmol) and the mixture was heated at reflux under an atmosphere of nitrogen until HPLC indicated that starting material was consumed. The reaction mixture was concentrated and the residue was dissolved in EtOAc (500 mL) and washed with 2M NaOH (aq) (100 mL) and brine. The organic phase was dried (Na₂SO₄) and then evaporated to dryness. The residue was purified on SiO₂ eluted with DCM and finally DCM:MeOH (98:2) to give 8.56 g (72%) of the title compound (94% pure, HPLC purity).

¹H NMR (CDCl₃) δ 9.87 (s, 1H), 7.76 (m, 2H), 7.72 (m, 1H), 7.55 (m, 2H), 7.14 (m, 1H), 6.84 (m, 1H). ¹³C NMR (CDCl₃) δ 185.5, 142.2, 129.4 (q, J=33 Hz), 129.0, 127.4 (q, J=4 Hz), 126.8, 123.8 (q, J=272 Hz), 122.1, 121.1, 110.5. MS (ESI) 240 (M+1H⁺).

### 3-Formyl-1-methyl-1H-indole-5-carbonitrile

1H-indole-5-carbonitrile (5.9 mmol, 1008 mg) was dissolved in 25 mL of THF and the solution was cooled to 0° C. under N₂-athmosphere. Sodium hydride (10.4 mmol, 250 mg) was added carefully in portions and iodomethane (9.6 mmol, 1368 mg) was added. The mixture was stirred at 0° C. for 1 h. More iodomethane (4.8 mmol, 684 mg) was added and the stirring continued for 45 min. The mixture was poured over ice and the resulting slurry was extracted with EtOAc. The organic layer was dried over Na₂SO₄, filtered and evaporated. This gave 1.0 g (92%) of the title product.

¹H NMR (500 MHz, MeOH-d₄) δ 9.94 (s, 1H), 8.55 (s, 1H), 8.26 (s, 1H), 7.71 (d, 1H), 7.65 (d, 1H), 3.98 (s, 3H)

### 5-Fluoro-1-methyl-1H-indole-3-carbaldehyde

5-Fluoro-1H-indole-3-carbaldehyde (6.1 mmol, 990 mg) was dissolved in 15 mL of THF and the solution was cooled to 0° C. under N₂-athmosphere. Sodium hydride (8.3 mmol, 200 mg) was added carefully in portions and iodomethane (8.1 mmol, 1150 mg) was added. The mixture was stirred at 0° C. for 1 h. More iodomethane (8.1 mmol, 1150 mg) was added and the stirring continued for 50 min. The mixture was poured over ice and the resulting slurry was extracted with EtOAc. The organic layer was dried over Na₂SO₄, filtered and evaporated. This gave 960 mg (89%) of the title product.

¹H NMR (400 MHz, CDCl₃) δ 9.96 (s, 1H), 8.27-8.22 (m, 1H), 7.66 (s, 1H), 7.11-7.02 (m, 2H), 3.84 (s, 3H)

### 5-Bromo-1-methyl-1H-indole-3-carbaldehyde

5-Bromo-1H-indole-3-carbaldehyde (4.8 mmol, 1076 mg) was dissolved in 15 mL of THF and the solution was cooled to 0° C. under N₂-athmosphere. Sodium hydride (11.7 mmol, 280 mg) was added carefully in portions and iodomethane (8.1 mmol, 1150 mg) was added. The mixture was stirred at 0° C. for 1 h. More iodomethane (8.1 mmol, 1150 mg) was added and the stirring continued for 50 min. The mixture was poured over ice and the resulting slurry was extracted with EtOAc. The organic layer was dried over Na₂SO₄, filtered and evaporated. This gave 1037 mg (91%) of the title product.

1H NMR (400 MHz, MeOH-d₄) δ 9.82 (s, 1H), 8,29 (m, 1H), 8.06 (s, 1H), 7.43 (m, 2H), 3.89 (s, 3H)

### 5-chloro-3-formyl-N,N-dimethyl-1H-indole-1-carboxamide

5-Chloro-1H-indole-3-carbaldehyde (1.5 g, 8.35 mmol) in THF (40 mL) was added drop wise to a solution of NaH (0.24 g, 10.0 mmol) in THF (10 nmL) at room temperature under an atmosphere of nitrogen. After 10 minutes dimethylcarbamyl chloride (1.26 g, 11.7 mmol) was added drop wise to the mixture and the mixture was stirred at r.t. until HPLC indicated that starting material was consumed. Water was added to the mixture, the THF was removed by evaporation and the aq. layer was extracted with CH₂Cl₂. The combined organic layers was evaporated to dryness. Purification of the residue by flash chromatography eluting with CH₂Cl₂ (100%) to CH₂Cl₂:MeOH (99:1) gave an oily residue. The residue was dissolved in CH₂Cl₂ and washed with sat. aq. Na₂CO₃ and evaporated to give 1.74 g (6.9 mmol, 83% yield) of the title compound.

¹H NMR (MeOD-d₄) δ 9.95 (s, 1H), 8.33 (s, 1H), 8.14 (d, 1H), 7.57 (d, 1H), 7.32 (dd, 1H), 3.10 (s, 6H). ¹³C NMR(MeOD-d₄) δ 187.4, 154.3, 140.2, 136.1, 130.7, 127.3, 126.5, 122.2, 120.8, 115.8, 38.6 MS (ESI) 251.1 (M+H⁺).

### 5-chloro-1-(methylsulfonyl)-1H-indole-3-carbaldehyde

5-Chloro-1H-indole-3-carbaldehyde (1.5 g, 8.35 mmol) in THF (50 mL) was added drop wise to a solution of NaH dispersed in mineral oil (0.24 g, 10.0 mmol) in THF (20 mL) at room temperature under an atmosphere of nitrogen. After 10 minutes methanesulfonyl chloride (1.34 g, 11.7 mmol) was added drop wise to the mixture and the mixture was stirred at r.t. until HPLC indicated that starting material was consumed. Water was added to the mixture and the THF was removed by evaporation. The aq. layer was extracted with CH₂Cl₂ and the organic layer was evaporated to dryness. The resulting reddish residue was washed with Et₂O to give 0.50 g of the title compound as a solid.

¹H NMR (DMSO-d₆) δ 10.09 (s, 1H), 8.70 (s, 1H), 8.16 (d, 1H), 7.93 (d, 1H), 7.55 (dd, 1H), 3.71 (s, 3H). ¹³C NMR (DMSO-d₆) δ 186.8, 139.6, 133.2, 129.5, 126.9, 125.9, 120.8, 119.7, 115.0, 41.7.

### (1S,3S)-N-(7-methoxy-4-methylquinolin-2-yl)cyclohexane-1,3-diamine

### a) Benzyl {(1S,3S)-3-[benzyloxycarbonyl-(7-methoxy-4-methylquinolin-2-yl)amino]cyclohexyl}carbamate

A mixture of (1S,3S)-Dibenzyl-cyclohexane-1,3-diylbiscarbamate (0.106 g, 0.28 mmol), 2-chloro-7-methoxy-4-methylquinoline (0.057 g, 0.27 mmol), Pd(OAc)₂ (0.006 g, 0.03 mmol), BINAP (0.017 g, 0.03 mmol), Cs₂CO₃ (0.267 g, 0.82 mmol)) in toluene (1 mL) under an atmosphere of N₂ was stirred at 70° C. for 24 h. The reaction mixture was cooled to rt, diluted with EtOAc/MeOH 10:1, filtered through a short plug of silica and concentrated. The residue was purified on a Biotage Horizon silica cartridge (gradient heptane, 10% EtOAc→100% EtOAc) to give 0.126 g (66%) of the title compound.

¹H NMR (400 MHz, MeOH-d₄) δ 7.87 (d, J=9.1 Hz, 1H), 7.31-7.15 (m, 12H), 6.97 (s, 1H), 5.09-5.01 (m, 4H), 4.49-4.39 (m, 1H), 3.90 (bs, 1H), 3.85 (s, 3H), 2.56 (s, 3H), 2.14 (bd, J=13.3 Hz, 1H), 1.99 (bd, J=11.5 Hz, 1H), 1.75-1.20 (m, 6H); ¹³C NMR (101 MHz, MeOH-d₄) δ 162.5, 158.1, 156.5, 153.5, 149.8, 148.5, 138.4, 137.7, 129.4, 129.0, 128.9, 128.8, 128.7, 126.2, 123.5, 122.1, 120.6, 108.1, 68.3, 67.2, 56.0, 55.6, 48.7, 36.4, 32.2, 30.2, 21.4, 18.7; LC-MS [M+H]⁺ 554.2.

### b) (1S,3S)-N-(7-methoxy-4-methylquinolin-2-yl)cyclohexane-1,3-diamine

Benzyl {(1S,3S)-3-[benzyloxycarbonyl-(7-methoxy-4-methylquinolin-2-yl)amino]cyclohexyl}carbamate (0.126 g, 0.18 mmol) and 10% Pd on activated carbon (0.020 g) in ethanol (5 mL) was stirred under an atmosphere of H₂. After 4 h, the mixture was filtered through Celite and concentrated. The residue was purified on an Isolute 2 g Flash Si column eluted with EtOAc/MeOH 5:1, 1% NEt₃ to yield 0.047 g (90%) of the title compound.

¹H NMR (400 MHz, MeOH-d₄) δ 7.61 (d, J=9.1 Hz, 1H), 7.04 (d, J=2.4 Hz, 1H), 6.80 (dd, J=9.1, 2.6 Hz, 1H), 6.49 (d, J=0.8 Hz, 1H), 4.34-4.28 (m, 1H), 3.85 (s, 3H), 3.09-3.02 (m, 1H), 2.45 (d, J=0.8 Hz, 3H), 1.96-1.88 (m, 1H), 1.80-1.55 (m, 6H), 1.36-1.26 (m, 1H); ¹³C NMR (101 MHz, MeOH-d₄) δ 162.2, 158.6, 150.8, 146.1, 125.8, 119.4, 113.7, 111.3, 106.4, 55.7, 47.1, 46.6, 40.4, 35.1, 32.0, 20.7, 18.7.

### EXAMPLES

### Example 1

### N,N-dimethyl-2-[(3-{[(5-pyridin-2-yl-2-thienyl)methyl]amino}cyclohexyl)amino]quinoline-4-carboxamide

### a) 2-Chloroquinoline-4-carbonyl chloride

2-chloroquinoline-4-carboxylic acid (0.5 g, 2.4 mmol) was slurried in 5 mL of DCM. Oxalyl chloride (0.41 mL, 4.8 mmol) was added and the reaction was started by the addition of two drops of DMF. The reaction mixture was stirred at room temperature over night. The solvent was evaporated to yield a brown solid (0.575 g) which was used without further purification.

### b) 2-Chloro-N,N-dimethylquinoline-4-carboxamide

2-chloroquinoline-4-carbonyl chloride (4.4 g, 19.5 mmol) was added to an ice-cold solution of dimethyl amine hydrochloride (1.6 g, 19.5 mmol) in Et₃N (5.4 mL) and DCM (46 mL). The ice bath was removed and the reaction mixture was stirred at room temp for 2.5 h and was then diluted with 150 mL of DCM. After washing with water and brine, the solution was dried over Na₂SO₄, filtered and evaporated. Flash chromatography (SiO₂, EtOAc) gave a brownish, solid compound (4.2 g, 91%).

¹H NMR (400 MHz, CDCl₃) δ 8.02 (d, 1H), 7.70-7.77 (m, 2H), 7.57 (m, 1H), 7.30 (s, 1H), 3.22 (s, 3H), 2.82 (s, 3H). LC-MS [M+H]⁺ 234.9, 236.8.

### c) 2-[(3-Aminocyclohexyl)aminol-N,N-dimethylquinoline-4-carboxamide

2-chloro-N,N-dimethylquinoline-4-carboxamide (0.42 g, 1.79 mmol) and cyclohexane-1,3-diamine (0.82 g, 7.2 mmol) were dissolved in pyridine (4 mL) and the solution was heated in a microwave oven at 175° C. for 20 minutes. The solvent was removed and the residue was purified using flash chromatography (SiO₂, 5:1 EtOAc:MeOH with 1% Et₃N) to yield the title compound as a mixture of stereoisomers (171 mg, 31%).

¹H NMR (400 MHz, MeOH-d₄; mixture of diastereomers) δ 7.63 (d, 1H), 7.52 (m, 1H), 7.41 (m, 1H), 7.20 (m, 1H), 6.72 (s, 1H, minor), 6.62 (s, 1H, major), 4.43 (m, 1H, minor), 4.03 (m, 1H, major), 3.191 (s, 3H, minor), 3.187 (s, 3H, major), 3.09 (m, 1H, minor), 2.86 (s, 3H), 2.85 (m, 1H, major), 2.27-2.34 (m, 1H), 1.40-2.15 (m, 7H). LC-MS [M+H]⁺ 313.1

### d) N,N-dimethyl-2-[(3-{[(5-pyridin-2-yl-2-thienyl)methyl]amino}cyclohexyl)aminol quinoline-4-carboxamide

Pol-BH₃CN (146 mg, ca 0.60 mmol) was suspended (swollen) in 0.6 mL of DCM. 2-[(3-aminocyclohexyl)amino]-N,N-dimethylquinoline-4-carboxamide (42 mg, 0.13 mmol) was dissolved in 1.2 mL of DCM:MeOH 1:1 and was mixed with a solution of 5-pyridin-2-ylthiophene-2-carbaldehyde (20 mg, 0.11 mmol) in 0.6 mL of DCM.

The combined solution was added to the polymer bound reducing agent and 0.06 mL of HOAc was added. The mixture was heated in a microwave oven at 100° C. for 10 minutes. The solution was cooled, filtered, evaporated and re-dissolved in DCM (1 mL). Aldehyde Wang resin (0.10 g, loading 2.66 mmol/g) was added and the mixture was stirred at room temperature for 24 hours. The polymer was filtered off and was washed with DCM:MeOH 1:1. The combined solutions was applied to a 1 g Isolute SCX-2 ion exchange column which was washed with 10 mL of MeOH. Elution with 7 mL of 10% Et₃N in MeOH gave the crude title product, which was further purified by flash chromatography (SiO₂, DCM:MeOH 9:1) to yield the title compound as a mixture of stereoisomers (36 mg, 55%).

¹H NMR (400 MHz, MeOH-d₄; mixture of diastereomers) δ 8.43 (m, 1H, major), 8.40 (m, 1H, minor), 7.38.7.80 (m, 6H), 7.14-7.23 (m, 2H+1H, minor), 7.01 (d, 1H, major), 6.90 (s, 1H, minor), 6.62 (s, 1H, major), 4.43 (m, 1H, minor), 4.00 (s, 2H, and m, 1H, major), 3.17 (s, 3H, major), 3.16 (s, 3H, minor), 2.97 (m, 1H, minor), 2.83 (s, 3H, major), 2.81 (s, 3H, minor), 2.76 (m, 1H, major), 2.40 (m, 1H), 1.40-2.10 (m, 7H). ¹³C NMR (101 MHz, MeOH-d₄, major isomer) δ 169.6, 156.2, 152.7, 149.0, 148.4, 145.6, 143.7, 143.2, 137.3, 130.0, 127.0, 125.8, 125.1, 124.2, 122.3, 122.0, 119.1, 119.0, 110.2, 54.4, 45.6, 44.6, 38.9, 37.9, 33.7, 32.5, 31.7, 22.9. LC-MS [M+H]⁺ 486.2, 487.2

### Example 2

### (1S,3S)-N-(6-chloro-4-methylquinolin-2-yl)-N′-[(1-methyl-1H-indol-3-yl)methyl]cyclohexane-1,3-diamine

### a) Benzyl {(1S,3S)-3-[benzyloxycarbonyl-(6-chloro-4-methylquinolin-2-yl)amino]cyclohexyl}carbamate

(1S,3S)-Dibenzyl-cyclohexane-1,3-diylbiscarbamate (406 mg, 1.00 mmol), 2,6-dichloro-4-methylquinoline (270 mg, 1.27 mmol), palladium(II)acetate (23 mg, 0.10 mmol), BINAP (800 mg, 1.28 mmol), Cs₂CO₃ (830 mg, 2.55 mmol) and 3.5 mL toluene was sealed under nitrogen in a vial. The mixture was heated at 70° C. for 48 h. DCM was added and the mixture was washed with water (3×50 mL). The organic phase was dried over Na₂SO₄ and evaporated. The residue was purified on a pre-packed SiO₂-column (Isolute, 50 g) eluted with DCM:EtOAc 10:2 to yield 530 mg (80%) of the title compound.

1H NMR (500 MHz, CDCl₃) δ 7.92 (m, 2H), 7.61 (dd, 1H), 7.38-7.21 (m, 10H), 7.11 (s, 1H), 5.18 (bd, 2H), 5.11 (bd, 2H), 4.41 (m, 1H), 4.05 (m, 1H), 2.63 (s, 3H), 2.13-1.35 (m, 8H).

### b) (1S,3S)-N-(6-chloro-4-methylquinolin-2-yl)cyclohexane-1,3-diamine

Benzyl {(1S,3S)-3-[benzyloxycarbonyl-(6-chloro-4-methylquinolin-2-yl)amino]cyclohexyl}carbamate (530 mg, 0.85 mmol) was hydrogenated at rt and 1 atm for 6 h with 10% Pd-C 50% water (160 mg) in ethanol (30 mL). The catalyst was filtered off through hyflo. Since there were still 30% starting material left, the hydrogenation was restarted with fresh catalyst (80 mg). After 3 h all starting material was consumed. The catalyst was filtered off through hyflo and the solvent was evaporated. The residue was purified on a pre-packed SiO₂-column (Isolute, 20 g) eluted with DCM:MeOH (containing 1% NH₂OH aq) 10:1 to yield 160 mg (59%) of the title compound.

¹H NMR (500 MHz, CDCl₃) δ 7.69 (d, 1H), 7.57 (d, 1H), 7.43 (dd, 1H), 6.51 (s, 1H), 4.30 (m, 1H), 3.13 (m, 1H), 2.52 (s, 3H), 1.92-1.55 (m, 6H), 1.36-1.25 (m, 2H)

### c) (1S,3S)-N-(6-chloro-4-methylquinolin-2-yl)-N′-[(1-methyl-1H-indol-3-yl)methyl]cyclohexane-1,3-diamine

Pol-BH₃CN (506 mg, 2.67 mmol) was suspended in 1.2 mL of DCM. (1S,3S)-N-(6-chloro-4-methylquinolin-2-yl)cyclohexane-1,3-diamine (160 mg, 0.55 mmol) dissolved in 3 mL of MeOH:DCM 1:2, 1-methylindole-3-carbaldehyde (70 mg, 0.44 mmol) dissolved in 1.2 mL MeOH:DCM 1:1 and 0.16 mL HOAc was added. The mixture was heated in a microwave oven at 100° C. for 10 minutes. The reaction mixture was cooled, filtered and the solvent was evaporated. The residue was first purified on a pre-packed SiO₂-column (Isolute, 20 g) eluted with DCM:MeOH (containing 1% NH₄OH aq) 10:1. The compound was further purified on HPLC (C8-column 250×20, gradient 0.1M NH₄OAc, 5% CH₃CN to 100% CH₃CN). After freeze-drying the pure fractions 87 mg (36%) of the title compound was obtained.

¹H NMR (400 MHz, MeOH-d₄) δ 7.72 (d, 1H), 7.59-7.54 (m, 2H), 7.42 (dd, 1H), 7.27 (d, 1H), 7.17 (s, 1H), 7.14 (t, 1H), 6.96 (t, 1H), 6.64 (s, 1H), 4.43 (m, 1H), 4.34 (s, 2H), 3.58 (s, 3H), 3.37 (m, 1H), 2.56 (bd, 1H), 2.45 (s, 3H), 2.12 (bd, 1H), 1.85-1.54 (m, 6H). ¹³C NMR (101 MHz, MeOH-d₄) δ 156.5, 145.7, 144.5, 137.2, 130.8, 129,5, 127.2, 127.1, 126,8, 124,5, 122.8, 122.1, 119.8, 118.0, 113.8, 109.5, 103.7, 51.9, 45.5, 38.8, 32.2, 31.8, 29.5, 28.7, 19.3, 17.5. LC-MS [M+H]⁺ 433.2

### Example 3 and 4

### (1S,3S)-N-(6-fluoro-4-methylquinolin-2-yl)-N′-(3-thienylmethyl)cyclohexane-1,3-diamine and

### (1R,3R)-N-(6-fluoro-4-methylquinolin-2-yl)-N′-(3-thienylmethyl)cyclohexane-1,3-diamine

The title compounds (435 mg) was prepared as an enantiomerically enriched mixture (˜20% ee) by a method analogous to that described for Example 2 starting from dibenzyl trans-cyclohexane-1,3-diylbiscarbamate (˜20% ee) and the enantiomers were separated on a Chiralcel OJ column (250×20 mm i.d.) using MeOH:Et₃N 100:0.1 as eluent. The collected fractions containing the pure enantiomers were evaporated, solvents were removed and each residue was re-dissolved in CH₃CN/H₂O and freeze dried. The enantiomeric ratio in the starting material is maintained in the products. Thus, the absolute configuration of the major enantiomer was assumed to be (1S,3S).

### (1S,35)-N-(6-fluoro-4-methylquinolin-2-yl)-N′-(3-thienylmethyl)cyclohexane-1,3-diamine major enantiomer (158 mg, 99.2% ee)

¹H NMR (400 MHz, CDCl₃) δ 7.60 (dd, 1H), 7.36 (dd, 1H), 7.23-7.29 (m, 2H), 7.10 (m, 1H), 7.05 (m, 1H), 6.51 (s, 1H), 4.63 (m, 1H), 4.31 (m, 1H), 3.85 (s, 2H), 2.94 (m, 1H), 2.50 (s, 3H), 1.40-1.95 (m, 9H). ¹³C NMR (101 MHz, CDCl₃) δ 159.3, 156.9, 155.9, 145.2, 144.6, 142.0, 128.5, 127.8, 125.9, 124.0, 121.6, 118.7, 118.5, 112.2, 107.9, 107.7, 52.1, 46.4, 46.2, 37.6, 32.1, 31.9, 20.1, 19.1. LC-MS [M+H]⁺ 370.2 [α]_{D}=−130.7° (c 1, MeOH)

### (1R,3R)-N-(6-fluoro-4-methylquinolin-2-yl)-N′-(3-thienylmethyl)cyclohexane-1,3-diamine minor enantiomer (101 mg, 98.4% ee) LC-MS [M+H]⁺ 370.2 [α]_{D}=+125.6° (c 1, MeOH)

### Example 5

### (1S3S)-N-(6-fluoro-4-methoxyquinolin-2-yl)-N′-(3-thienylmethyl)cyclohexane-1,3-diamine

Starting from dibenzyl (1S,3S)-cyclohexane-1,3-diylbiscarbamate (described earlier) and 2-chloro-6-fluoro-4-methoxyquinoline (described earlier), the title compound (56 mg) was prepared by a method analogous to that described for Example 2.

¹H NMR (400 MHz, MeOH-d₄) δ 7.45-7.55 (m, 2H), 7.19-7.7.28 (m, 2H), 7.09 (m, 1H), 7.03 (m, 1H), 6.23 (s, 1H), 4.35 (m, 1H), 3.94 (s, 3H), 3.78 (s, 2H), 2.86 (m, 1H), 2.05 (m, 1H), 1.55-1.85 (m, 6H), 1.36 (m, 1H). ³C NMR (101 MHz, MeOH-d₄) δ 162.1, 158.9, 158.1, 156.6, 145.5, 140.5, 127.7, 126.5, 125.4, 122.0, 118.4, 118.2, 117.9, 105.7, 105.5, 90.9, 55.0, 51.1, 45.7, 44.9, 35.9, 31.2, 30.9, 19.7. LC-MS [M+H]⁺ 386.2

### Example 6

### (1S,3S)-N-(6-fluoro-4-methylquinolin-2-yl)-N′-[(1-methyl-1H-indol-3-yl)methyl]cyclopentane-1,3-diamine

### a) Tert-butyl {(1S,3S)-3-[(6-fluoro-4-methylquinolin-2-yl)amino]cyclopentyl}carbamate

A mixture of 2-chloro-6-fluoro-4-methylquinoline (0.54 g, 2.76 mmol), tert-butyl [(1S,3S)-3-aminocyclopentyl]carbamate (0.69 g, 3.45 mmol), Cs₂CO₃ (2.02 g, 6.21 mmol), palladium(II) acetate (43 mg, 0.193 mmol), and BINAP (0.12 g, 0.193 mmol) in dioxane (10 mL) was heated and stirred at 80° C. for 5 h. The reaction mixture was cooled to room temperature, filtered through a plug of celite and the plug washed with EtOAc and MeOH. The combined filtrate was concentrated and the residue purified by flash chromatography and eluted with heptane:EtOAc 1:1. Yield: 434 mg (44%)

¹H NMR (400 MHz, MeOH-d₄) δ 7.59 (dd, 1H), 7.39 (dd, 1H), 7.25 (m, 1H), 6.60 (s, 1H), 4.44 (m, 1H), 4.03 (m, 1H), 2.44 (d, J=0.8 Hz, 3H), 1.80-2.30 (m, 4H), 1.45-1.55 (m, 2H), 1.43 (s, 9H). LC-MS [M+H]⁺ 360.3

### b) (1S,3S)-N-(6-fluoro-4-methylquinolin-2-yl)cyclopentane-1,3-diamine

tert-butyl {(1S,3S)-3-[(6-fluoro-4-methylquinolin-2-yl)amino]cyclopentyl}carbamate (0.434 g, 1.21 mmol) was dissolved in DCM (4.6 mL). TFA (2.3 ml) was added and the mixture was stirred at room temperature for 4 h. The pH of the solution was adjusted to about 10 with 2M NaOH (aq.) and the aqeous solution was extracted with EtOAc. The combined organic phase was dried over Na₂SO₄, filtered and evaporated. The resulting product (0.39 g) was used in the subsequent step without further purification.

LC-MS [M+H]⁺ 260.2

### c) (1S,3S)-N-(6-fluoro-4-methylquinolin-2-yl)-N′-[(1-methyl-1H-indol-3-yl)methyl]cyclopentane-1,3-diamine

The title compound (387 mg) was prepared from (1S,3S)-N-(6-fluoro-4-methylquinolin-2-yl)cyclopentane-1,3-diamine and 1-methyl-1H-indole-3-carbaldehyde by a method analogous to that described for Example 1 (step d).

¹H NMR (400 MHz, MeOH-d₄) δ 7.55-7.60 (m, 2H), 7.36 (dd, 1H), 7.27 (d, 1H), 7.23 (m, 1H), 7.13 (m, 1H), 7.07 (s, 1H), 7.01 (m, 1H), 6.58 (s, 1H), 4.46 (m, 1H), 3.88 (s, 2H), 3.70 (s, 3H), 3.35 (m, 1H), 2.42 (d, J=0.8 Hz, 3H), 2.05-2.30 (m, 2H), 1.85-1.98 (m, 2H), 1.43-1.58 (m, 2H). ¹³C NMR (101 MHz, MeOH-d₄) δ 159.1, 156.8, 144.8, 144.1, 137.3, 127.8, 127.2, 127.1, 123.9, 121.4, 118.8, 118.3, 117.7, 117.5, 113.5, 112.1, 109.1, 107.6, 107.4, 56.9, 51.0, 42.2, 39.5, 31.7, 31.5, 31.0, 17.5. LC-MS [M+H]⁺ 403.2

### Example 7

### N-(6-chloroquinolin-2-yl)-N′-(3-thienylmethyl)cyclohexane-1,3-diamine

### a) N-(6-chloroquinolin-2-yl)cyclohexane-1,3-diamine

2,6-dichloroquinoline (198 mg, 1.0 mmol,) and cyclohexane-1,3-diamine (457 mg, 4.0 mmol) were refluxed in pyridine (10 mL) for 48 h. The solvent was evaporated and the residue was purified on a pre-packed SiO₂-column (Isolute, 10 g) eluted with DCM:MeOH (containing 1% NH₂OH aq) 10:1 to yield 100 mg (36.3%) of the title compound.

¹H NMR (500 MHz, MeOH-d₄) δ 7.72-7.67 (m, 1H), 7.55-7.50 (m, 2H), 7.41-7.38 (m, 1H), 6.80 (d, 1H, minor isomer), 6.71 (d, 1H, major isomer), 4.38 (bs, 1H, minor isomer), 3.98 (m, 1H, major isomer), 3.04 (m, 1H, minor isomer), 2.79 (m, 1H, major isomer), 2.25-1.01 (m, 8H).

### b) N-(6-chloroquinolin-2-yl)-N′-(3-thienylmethyl)cyclohexane-1,3-diamine

Pol-BH₃CN, (190 mg, 1.0 mmol) was suspended in 0.6 mL of DCM. N-(6-chloroquinolin-2-yl)cyclohexane-1,3-diamine (55 mg, 0.2 mmol) dissolved in 1.2 mL of MeOH:DCM 3:1, thiophene-3-carbaldehyde (22 mg, 0.2 mmol) dissolved in 0.6 mL MeOH:DCM 1:1 and 0.06 mL HOAc were added. The mixture was heated in a microwave oven at 100° C. for 10 minutes. The reaction mixture was cooled, filtered and evaporated. The residue was purified on a pre-packed SiO₂-column (Isolute, 5 g) eluted with DCM:MeOH (containing 1% NH₂OH aq) 10:0.5 to yield 20 mg (26%) of the title compound as a mixture of stereoisomers.

¹H NMR (500 MHz, MeOH-d₄) δ 7.75-7.70 (m, 1H), 7.58-7.52 (m, 2H), 7.43-7.38 (m, 1H), 7.37-7.35 (m, 1H, major isomer), 7.29-7.27 (m, 1H, minor isomer), 7.27-7.25 (m, 1H, major isomer), 7.14 (m, 1H, minor isomer), 7.11 (dd, 1H, major isomer), 7.05 (dd, 1H, minor isomer), 6.8 (d, 1H, minor isomer) 6.72 (d, 1H, major isomer), 4.4 (m, 1H, minor isomer), 3.98 (m, 1H, major isomer) 3.83 (s, 2H, major isomer) 3.81 (s, 2H, minor isomer), 2.89 (m, 1H, minor isomer), 2.69 (m, 1H, major isomer), 2.41-1.06 (m, 8H). ¹³C NMR (125.6 MHz, MeOH-d₄) δ 157.1, 146.5, 140.3, 136.0, 129.4, 127.7, 126.7, 126.5, 126.2, 125.6, 124.1, 122.2, 114.4, 54.8, 44.9, 38.9, 32.6, 31.6, 22.9, 19.7

### Example 8

### N-(6-chloroquinolin-2-yl)-N]-[(1-methyl-1H-pyrrol-2-yl)methyl[cyclohexane-1,3-diamine

Pol-BH₃CN (190 mg, 1.0 mmol) was suspended in 0.6 mL of DCM. N-(6-chloroquinolin-2-yl)cyclohexane-1,3-diamine (55 mg, 0.2 mmol, from Example 7 Step a) dissolved in 1.2 mL of MeOH:DCM 3:1, 1-methylpyrrole-2-carbaldehyde (22 mg, 0.2 mmol) dissolved in 0.6 mL MeOH:DCM 1:1 and 0.06 mL HOAc was added. The mixture was heated in a microwave oven at 100° C. for 10 minutes. The reaction mixture was cooled, filtered and evaporated. The residue was purified on a pre-packed SiO₂-column (Isolute, 5 g) eluted with DCM:MeOH 10:2 to yield 30 mg (36%) of the title compound as a mixture of stereoisomers.

¹H NMR (300 MHz, MeOH-d₄) δ 7.8-7.73 (m, 1H), 7.62-7.52 (m, 2H), 7.47-7.39 (m, 1H), 6.87-6.3 (m, 2H), 6.28 (m, 1H, major isomer), 6.16 (m, 1H, minor isomer), 6.06 (t, 1H, major isomer), 5.97 (t, 1H, minor isomer), 4.48 (m, 1H, minor isomer), 4.19 (s, 2H, major isomer), 4.16 (s, 2H, minor isomer), 4.07 (m, 1H, major isomer), 3.67 (s, 3H, major isomer), 3.61 (s, 3H, minor isomer), 3.42-3.18 (m, 1H), 2.70-1.23 (m, 8H). ¹³C NMR (75 MHz, MeOH-d₄) δ 156.8, 146.3, 136.1, 129.5, 126.9, 126.6, 126.5, 126.2, 124.1, 123.5, 114.3, 111.2, 107.4, 55.4, 52.9, 39.6, 36.2, 32.9, 31.8, 29.1, 22.8. LC-MS [M+H]⁺ 369.0

### Example 9

### N-(6-chloroquinolin-2-yl)-N′-(quinolin-3-ylmethyl)cyclohexane-1,3-diamine

Pol-BH₃CN, (1 mmol, 190 mg) was suspended in 0.6 mL of DCM. N-(6-chloroquinolin-2-yl)cyclohexane-1,3-diamine (0.2 mmol, 55 mg, prepared as described in Example 7 Step a) dissolved in 1.2 mL of MeOH:DCM3:1, quinoline-3-carbaldehyde (0.2 mmol, 31 mg) dissolved in 0.6 mL MeOH:DCM 1:1 and 0.06 mL HOAc was added. The mixture was heated in a microwave oven at 100° C. for 10 minutes. The reaction mixture was cooled, filtered and evaporated. The residue was purified on a pre-packed SiO₂-column (Isolute, 5 g) eluted with DCM:MeOH 20:3 to yield 27 mg (32%) of the title compound as a mixture of stereoisomers.

¹H NMR (300 MHz, MeOH-d₄) δ 8.9 (m, 1H, major isomer) 8.87 (m, 1H, minor isomer), 8.38 (m, 1H, major isomer), 8.26 (m, 1H, minor isomer), 8.08-7.90 (m, 2H), 7.82-7.50 (m, 5H), 7.42 (d, 1H, major isomer), 7.39 (d, 1H, minor isomer), 6.78-6.70 (m, 1H), 4.44 (m, 1H, minor isomer), 4.21 (s, 2H, major isomer), 4.19 (s, 2H, minor isomer), 4.04 (m, 1H, major isomer), 3.16 (m, 1H, minor isomer), 3.02 (m, 1H, major isomer), 2.64-1.16 (m, 8H) ¹³C NMR (75 MHz, MeOH-d₄) δ 156.8, 151.1, 146.9, 146.3, 137.1, 136.0, 130.2, 130.0, 129.5, 128.1, 128.0, 127.8, 127.3, 126.5, 126.4, 126.2, 124.1, 114.3, 55.8, 37.8, 32.3, 30.6, 22.9, 22.4, 19.7 LC-MS [M+H]⁺ 417.00

### Example 10

### N⁶,N⁶-dimethyl-N²-{3-[(3-thienylmethyl)amino]cyclohexyl}quinoline-2,6-diamine

### a) N²-(3-aminocyclohexyl)-N⁶,N⁶-dimethylquinoline-2,6-diamine

2-chloro-N,N-dimethylquinolin-6-amine (100 mg, 0.48 mmol), cyclohexane-1.3-diamine (163 mg, 1.43 mmol), palladium(II)acetate (9 mg, 0.04 mmol), BINAP (18 mg, 0.06 mmol) and 2.5 mL toluene was sealed under nitrogen in a microwave vial. The mixture was heated in a microwave oven at 150° C. for 20 minutes. The reaction mixture was cooled, filtered and the solvent was evaporated. The residue was purified on a pre-packed SiO₂-column (Isolute, 10 g) eluted with DCM:MeOH (containing 1% NH₄OH aq) 10:2 to yield 45 mg (33%) of the title compound as a mixture of stereoisomers.

¹H NMR (500 MHz, MeOH-d₄) δ 7.69 (d, 1H), 7.52 (d, 1H), 7.22 (dd, 1H), 6.85 (d, 1H), 6.64 (d, 1H), 4.03 (m, 1H, minor isomer), 3.89 (m, 1H major isomer), 3.04 (m, 1H, minor isomer), 2.90 (s, 6H), 2.78 (m, 1H, major isomer), 2.27-0.98 (m, 8H)

### b) N⁶,N⁶-dimethyl-N²-{3-[(3-thienylmethyl)amino]cyclohexyl}quinoline-2,6-diamine

Pol-BH₃CN (190 mg, 1.0 mmol) was suspended in 0.6 mL of DCM. N²-(3-aminocyclohexyl)-N⁶,N⁶-dimethylquinoline-2,6-diamine (40 mg, 0.14 mmol) dissolved in 1.2 mL of MeOH:DCM 3:1, thiophene-3-carbaldehyde (20 mg, 0.17 mmol) dissolved in 0.6 mL MeOH/DCM 1:1 and 0.06 mL HOAc was added. The mixture was heated in a microwave oven at 100° C. for 10 minutes. The reaction mixture was cooled, filtered and the solvent was evaporated. The residue was first purified on a pre-packed SiO₂-column (Isolute, 5 g) eluted with DCM:MeOH (containing 1% NH₂OH aq) 10:1 yielding a crude product which was further purified by automated HPLC to give 30 g (54%) of the title compound.

¹H NMR (300 MHz, MeOH-d₄) δ 7.89 (d, 1H), 7.83-7.11 (m, 5H), 6.91 (d, 1H), 6.81 (d, 1H), 4.24 (s, 2H), 3.95 (m, 1H), 3.23 (m, 1H), 2.96 (s, 6H), 2.65-1.24 (m, 8H) LC-MS [M+H]381.16

### Example 11

### (1S,3S)-N-[(4-chloro-1-methyl-1H-pyrazol-3-yl)methyl]-N′-(6-methoxy-4-methylquinolin-2-yl)cyclopentane-1,3-diamine

Pol-BH₃CN (190 mg, 1 mmol) was suspended in 0.6 mL of DCM. (1S,3S)-N-(6-methoxy-4-methylquinolin-2-yl)cyclopentane-1,3-diamine (50 mg, 0.18 mmol) dissolved in 1.2 mL of MeOH:DCM 3:1, 4-chloro-1-methyl-1H-pyrazole-3-carbaldehyde (27 mg, 0.18 mmol) dissolved in 0.6 mL MeOH:DCM 1:1 and 0.06 mL HOAc was added. The mixture was heated in a microwave oven at 100° C. for 10 minutes. The reaction mixture was cooled, filtered and the solvent was evaporated. The residue was purified by automated HPLC to give 24.6 mg (33%) of the title compound.

¹H NMR (400 MHz, MeOH-d₄) δ 7.71 (s, 1H), 7.60 (d, 1H), 7.22 (dd, 1H), 7.18 (d, 1H), 6.71 (s, 1H), 4.48 (m, 1H), 4.05 (s, 2H) 3.88 (s, 3H), 3.86 (s, 3H), 3.69 (m, 1H), 2.55 (s, 3H), 2.38-2.26 (m, 2H), 2.21-2.05 (m, 2H), 1.78-1.63 (m, 2H). ¹³C NMR (101 MHz, MeOH-d₄) δ 156.9, 155,6, 148.00, 143.2, 140.3, 131.1, 125,3, 124.8, 121.7, 113.6, 110.1, 105.5, 58.2, 56,1, 52.4, 41.9, 39.8, 38.3, 32.3, 30.1, 19.2. LC-MS [M+H]⁺ 400.4

### Example 12

### (1S,3S)-N-(6-methoxy-4-methylquinolin-2-yl)-N′-(1,2,3-thiadiazol-4-ylmethyl)cyclopentane-1,3-diamine

The title compound (31 mg) was prepared using the procedure described for the preparation of Example 11.

¹H NMR (400 MHz, MeOH-d₄) δ 8.97 (s, 1H), 7.62 (d, 1H), 7.25 (dd, 1H), 7.18 (d, 1H), 6.76 (s, 1H), 4.90 (s, 2H), 4.53 (m, 1H), 3.88 (s, 3H), 3.59 (m, 1H), 2.56 (s, 3H), 2.40-2.20 (m, 2H), 2.19-2.20 (m, 2H), 1.75-1.63 (m, 2H). ¹³C NMR (101 MHz, MeOH-d₄) δ 160.3, 157.2, 154.8, 149.3, 138.3, 137.2, 124.5, 124.0, 122,0, 113.4, 105.8, 58.4, 56.1, 52.7, 44.7, 39.1, 32.4, 30.9, 19.3. LC-MS [M+H]⁺ 370.4.

### Example 13

### (1S,3S)-N-(6-methoxy-4-methylquinolin-2-yl)-N′-[(5-pyridin-2-yl-2-thienyl)methyl]cyclopentane-1,3-diamine

The title compound (34 mg) was prepared using the procedure described for the preparation of Example 11

¹H NMR (400 MHz, MeOH-d₄) δ 8.48 (d, 1H), 7.84-7.77 (m, 2H), 7.62-7.58 (m, 2H), 7.29-7.17 (m, 4H), 6.70 (s, 1H), 4.5 (m, 1H), 4.29 (s, 2H), 3.88 (s, 3H), 3.69 (m, 1H), 2.55 (s, 3H), 2.36-2.27 (m, 2H), 2.21-2.03 (m, 2H), 1.80-1.65 (m, 2H). ¹³C NMR (101 MHz, MeOH-d₄) δ 156.9, 155.7, 150.3, 147.8, 146.7, 139.9, 138.6, 130.9, 126.3, 125.5, 124.8, 123.7, 121.6, 120.4, 113.8, 105.5, 58.0, 56.1, 52.4, 46.1, 38.5, 32.3, 30.3, 19.1, 15.4, 5.4. LC-MS [M+H]⁺ 445.5.

### Example 14

### (1S,3S)-N-({1-[(2-chloro-1,3-thiazol-5-yl)methyll-1H-indol-3-yl]methyl)-N′-(6-methoxy-4-methylquinolin-2-yl)cyclopentane-1,3-diamine

The title compound (31 mg) was prepared using the procedure described for the preparation of Example 11.

¹H NMR (400 MHz, MeOH-d₄) δ 7.74 (d, 1H), 7.59 (d, 1H), 7.55 (d, 2H), 7.51 (d, 1H), 7.30-7.14 (m, 4H), 6.68 (s, 1H), 5.59 (s, 2H), 4.51 (m, 1H), 4.39 (s, 2H), 3.88 (s, 3H), 3.81 (m, 1H), 2.54 (s, 3H), 2.43-2.11 (m, 4H), 1.90-1.66 (m, 2H). ¹³C NMR (101 MHz, MeOH-d₄) δ 156.8, 155.8, 153.0, 147.6, 140.9, 140.8, 139.4, 137.5, 130.5, 128.8, 125.8, 124.9, 124.0, 121.8, 121.6, 119.7, 113.7, 111.0, 107.6, 105.3, 57.9, 56.0, 52.2, 43.1, 41.9, 37.5, 32.1, 29.4, 19.1. LC-MS [M+H]⁺ 532.5.

### Example 15

### (1S,3S)-N-(6-methoxy-4-methylquinolin-2-yl)-N′-({5-[1-methyl-5-(trifluoromethyl)-1H-pyrazol-3-yl]-2-thienyl}methyl)cyclopentane-1,3-diamine

The title compound (33 mg) was prepared using the procedure described for the preparation of Example 11.

¹H NMR (400 MHz, MeOH-d₄) δ7.56 (d, 1H), 7.23 (d, 1H), 7.17 (dd, 1H), 7.15-7.10 (m, 2H), 6.70 (s, 1H), 6.64 (s, 1H), 4.45 (m, 1H), 4.08 (s, 2H), 3.97 (s, 3H), 3.86 (s, 3H), 3.46 (m, 1H), 2.50 (s, 3H), 2.34-2.13 (m, 2H), 2.05-1.91 (m, 2H), 1.65-1.52 (m, 2H). LC-MS [M+H]⁺ 516.5.

### Example 16

### (1S,3S)-N-(2,2′-bithien-5-ylmethyl)-N′-(6-methoxy-4-methylquinolin-2-yl)cyclopentane-1,3-diamine

Pol-BH₃CN (190 mg, 1.0 mmol) was suspended in 0.6 mL of DCM. (1S,3S)-N-(6-methoxy-4-methylquinolin-2-yl)cyclopentane-1,3-diamine (50 mg, 0.18 mmol) dissolved in 1.2 mL of MeOH:DCM 3:1, 2,2′-bithiophene-5-carbaldehyde (36 mg, 0.18 mmol) dissolved in 0.6 mL MeOH:DCM 1:1 and 0.06 mL HOAc was added. The mixture was heated in a microwave oven at 100° C. for 10 minutes. The reaction mixture was cooled, filtered and the solvent was evaporated. The residue was purified on a pre-packed SiO₂-column (Isolute, 5 g) eluted with DCM:MeOH 10:1 to yield 39 mg (44%) of the title compound.

¹H NMR (400 MHz, MeOH-d₄) δ 7.60 (d, 1H), 7.33 (dd, 1H), 7.22 (dd, 1H), 7.18 (dd, 1H), 7.16 (d, 1H), 7.11-7.07 (m, 2H), 7.02 (dd, 1H), 6.70 (s, 1H), 4.47 (m, 1H), 4.22 (s, 2H), 3.87 (s, 3H), 3.66 (m, 1H), 2.53 (s, 3H), 2.34-2.24 (m, 2H), 2.20-2.04 (m, 2H), 1.76-1.66 (m, 2H); LC-MS [M+H]⁺ 450.14

### Example 17

### N⁴,N⁴-dimethyl-N²-{3-[(3-thienylmethyl)amino]cyclohexyl}quinoline-2,4-diamine

### a) N²-(3-aminocyclohexyl)-N⁴,N⁴-dimethylquinoline-2,4-diamine

A mixture of 2-chloro-N,N-dimethylquinolin-4-amine (0.102 g 0.494 mmol), cyclohexane-1,3-diamine (0.141 g, 1.23 mmol), NaO^{t}Bu (0.017 g, 0.73 mmol), Pd(OAc)₂ (0.008 g, 0.034 mmol), and 2-(di-^{t}butylphosphino)biphenyl (0.017 g, 0.057 mmol) in toluene (1 mL) under an N₂-atmosphere was subjected to microwave heating single node 140° C., 10 min. The reaction mixture was cooled to room temperature, diluted with EtOAc:MeOH 5:1 containing 1% Et₃N and loaded on a short (˜2 cm) SiO₂ column. Elution with EtOAc:MeOH 5:1 containing 1% Et₃N gave 92 mg (65%) of the title compound as a mixture of diastereomers (˜5:1).

¹H NMR (400 MHz, MeOH-d₄) δ 7.81 (dd, 1H), 7.55 (bd, 1H), 7.40 (ddd, 1H), 7.11 (ddd, 1H), 6.26 (s, 1H, minor isomer), 6.15 (s, 1H, major isomer), 4.33 (m, 1H, minor isomer), 3.94 (tt, 1H, major isomer), 3.06 (m, 1H, minor isomer), 2.90 (s, 6H, minor isomer), 2.88 (s, 6H, major isomer), 2.81 (tt, 1H, major isomer), 2.26 (m, 1H, major isomer), 2.08-1.00 (m, 7H). LC-MS [M+H]⁺ 285.3.

### b) N⁴,N⁴-dimethyl-N²-{3-[(3-thienylmethyl)amino]cyclohexyl}quinoline-2,4-diamine

N⁴,N²-(3-aminocyclohexyl)-N⁴,N⁴-dimethylquinoline-2,4-diamine (0.036 g, 0.13 mmol) in DCM:MeOH 1:1 (1.2 mL), thiophene-3-carbaldehyde (0.11 mmol, 0.012 g) in DCM (0.6 mL) and HOAc (0.060 mL) was added to Pol-BH₃CN (0.15 g, 0.6 mmol, pre-swollen in DCM, 0.6 mL). The resultant mixture was subjected to microwave heating single node 100° C., 10 min. The resin was filtered off and washed with portions (1-2 mL) of DCM and MeOH, and the filtrate was concentrated. The residue was dissolved in DCM (1 mL), Pol-CHO (0.1 g, 0.3 mmol) added, and the slurry was stirred at room temperature for 16 h. The resin was filtered off and washed with portions (1-2 mL each) of DCM and MeOH. The filtrate was loaded on a 1 g Isolute SCX-2 ion exchange column, washed with MeOH (10 mL), and the product eluted with MeOH containing 10% Et3N to give 0.034 g (93%) of the title compound as a mixture of diastereomers (˜5:1).

¹H NMR (400 MHz, MeOH-d₄) δ 7.84-7.80 (m, 1H), 7.55 (bd, 1H), 7.43-7.38 (m, 1H), 7.34 (dd, 1H, major isomer), 7.26-7.02 (m, 3H), 6.24 (s, 1H, minor isomer), 6.15 (s, 1H, major isomer), 4.33 (m, 1H, minor isomer), 3.94 (tt, major isomer), 3.80 (s, 2H, major isomer), 3.78 (s, 2H, minor isomer), 2.90 (s, 6H, minor isomer), 2.88 (s, 6H, major isomer), 2.86 (m, 1H, minor isomer), 2.68 (tt, 1H, major isomer), 2.36 (m, 1H, major isomer), 2.08-1.04 (m, 7H). ¹³C NMR (101 MHz, MeOH-d₄, major isomer) δ 159.8, 159.0, 150.4, 141.6, 130.0, 128.9, 126.7, 126.2, 125.3, 123.3, 121.6, 120.5, 99.5, 55.9, 49.5, 46.1 44.2, 40.4, 34.1, 32.8, 24.1. LC-MS [M+H]⁺ 381.3.

### Example 18

### N⁴,N⁴-dimethyl-N²-[3-({[2-(phenylsulfonyl)-1,3-thiazol-5-yl]methyl}amino)cyclohexyl]quinoline-2,4-diamine

N²-(3-aminocyclohexyl)-N⁴,N⁴-dimethylquinoline-2,4-diamine (0.013 g, 0.046 mmol, see earlier) in DCM:MeOH 1:1 (0.6 mL), 2-(phenylsulfonyl)-1,3-thiazole-5-carbaldehyde (0.008 g, 0.03 mmol) in DCM (0.3 mL) and HOAc (0.030 mL) was added to Pol-BH₃CN (0.15 g, 0.6 mmol, pre-swollen in DCM, 0.3 mL). The resultant mixture was subjected to microwave heating single node 100° C. for 10 minutes. The resin was filtered off and washed with portions (1-2 mL) of DCM and MeOH, and the filtrate was concentrated. The residue was dissolved in DCM (1 mL), Pol-CHO (100 mg) added, and the slurry was stirred at room temperature for 16 h. The resin was filtered off and washed with portions (1-2 mL each) of DCM and MeOH. The filtrate was loaded on a 1 g Isolute SCX-2 ion exchange column, washed with MeOH (10 mL), and the product eluted with MeOH containing 10% Et₃N to give 0.010 g (61%) of the title compound as a mixture of diastereomers (˜5:1).

¹H NMR (400 MHz, MeOH-d₄) δ 8.06-7.08 (m, 10H), 6.21 (s, 1H, minor isomer), 6.14 (s, 1H, major isomer), 4.31 (m, 1H, minor isomer), 4.07 (s, 2H, major isomer), 4.05 (s, 2H, minor isomer), 3.91 (tt, 1H, major isomer), 2.90 (s, 6H), 2.87 (m, 1H, minor isomer), 2.66 (tt, 1H, major isomer), 2.33 (m, 1H), 2.04-1.01 (m, 7H). LC-MS [M+H]⁺ 522.2.

### Example 19

### N²-(3-{[(2,4-dimethoxypyrimidin-5-yl)methyl]amino}cyclohexyl)-N⁴,N⁴-dimethylquinoline-2,4-diamine

N²-(3-aminocyclohexyl)-N⁴,N⁴-dimethylquinoline-2,4-diamine (0.038 g, 0.13 mmol, see earlier) in DCM:MeOH 1:1 (1.2 mL), 2,4-dimethoxypyrimidine-5-carbaldehyde (0.019 g, 0.11 mmol) in DCM (0.6 mL) and HOAc (0.060 mL) was added to Pol-BH₃CN (0.15 g, 0.6 mmol, pre-swollen in DCM, 0.6 mL). The resultant mixture was subjected to microwave heating single node 100° C., 10 min. The resin was filtered off and washed with portions (1-2 mL) of DCM and MeOH, and the filtrate was concentrated. The residue was dissolved in DCM (1 mL), Pol-CHO (0.1 g, 0.3 mmol) added, and the slurry was stirred at room temperature for 16 h. The resin was filtered off and washed with portions (1-2 mL each) of DCM and MeOH. The filtrate was loaded on a 1 g Isolute SCX-2 ion exchange column, washed with MeOH (10 mL), and the product eluted with MeOH containing 10% Et₃N to give 0.041 g (83%) of the title compound as a mixture of diastereomers (˜5:1).

¹H NMR (400 MHz, MeOH-d₄) δ 8.16 (s, 1H, major isomer), 8.11 (s, 1H, minor isomer), 7.81 (dd, 1H, minor isomer), 7.80 (dd, 1H, major isomer), 7.56-7.52 (m, 1H), 7.40 (ddd, 1H), 7.14-7.08 (m, 1H), 6.23 (s, 1H, minor isomer), 6.15 (s, 1H, major isomer), 4.33 (m, 1H, minor isomer), 4.00-3.91 (m, 7H), 3.68 (s, 2H, major isomer), 3.65 (d, 2H, minor isomer), 2.90 (s, 6H, minor isomer), 2.88 (s, 6H, major isomer), 2.85 (m, 1H, minor isomer), 2.63 (tt, 1H, major isomer, 2.35 (m, 1H, major isomer), 2.06-1.02 (m, 7H). ¹³C NMR (101 MHz, MeOH-d₄, major isomer) δ 171.1, 166.1, 159.8, 159.0, 158.9, 150.4, 130.0, 126.3, 125.3, 121.6, 120.5, 113.9, 99.5, 56.2, 55.3, 54.7, 49.5, 44.2, 42.8, 40.6, 34.1, 32.9, 24.1. LC-MS [M+H]⁺ 437.3.

### Example 20

### 3-(6-Methoxy-4-methylquinolin-2-yl)-N-methyl-N-(3-thienylmethyl)-3-azabicyclo [3.2.1]octan-8-amine

### a) 3-(6-Methoxy-4-methylquinolin-2-yl)-N-methyl-3-azabicyclo[3.2.1]octan-8-amine

2-chloro-6-methoxy-4-methylquinoline (0.60 g, 2.89 mmol), tert-butyl 3-azabicyclo[3.2.1]oct-8-yl(methyl)carbamate (0.50 g, 2.07 mmol, from WO0147893), NaO^{t}Bu (0.32 g, 3.3 mmol), palladium(II) acetate (46 mg, 0.20 mmol) and BINAP (111 mg, 0.37 mmol) in dry toluene (4.5 mL) was heated in a microwave oven 135° C. for 15 minutes. The. reaction mixture was cooled to room temperature, filtered through a plug of Celite and the plug washed with EtOAc:MeOH 1:1 (500 mL). The combined filtrate was concentrated and the residue was purified by flash chromatography (SiO₂, EtOAc:n-heptane 1:2) to yield the intermediate, Boc-protected, derivative (130 mg) which was dissolved in 10 mL of EtOAc saturated with HCl(g). After stirring at room temperature for 1 h, the solvent was evaporated and the residue was dissolved in water. After washing with EtOAc, pH was adjusted to about using 2 M NaOH (aq.). The aqeous phase was extracted with EtOAc. The organic phase was washed with brine, dried with Na₂SO₄, filtered and concentrated to yield the title compound (76 mg, 12%)

¹H NMR (400 MHz, CDCl3) δ 7.63 (d, 1H), 7.18 (dd, 1H), 7.05 (d, 1H), 6.78 (s, 1H), 3.87 (m, 2H), 3.86 (s, 3H), 3.57 (br s, ˜2H), 3.33 (d, 2H), 2.85 (m, 1H), 2.52 (s, 3H), 2.48 (s, 3H), 2.31 (s br, 2H), 1.6-1.8 (m, 4H). LC-MS [M+H]⁺ 312.3, 313.3

### b) 3-(6-Methoxy4-methylquinolin-2-yl)-N-methyl-N-(3-thienylmethyl)-3-azabicyclo[3.2.1]octan-8-amine

Pol-BH₃CN (45 mg, ca 0.24 mmol) was suspended (swollen) in 0.3 mL of DCM. 3-(6-methoxy-4-methylquinolin-2-yl)-N-methyl-3-azabicyclo[3.2.1]octan-8-amine (42 mg, 0.134 mmol, from Step a) and thiophene-3-carbaldehyde (18 mg, 0.16 mmol) were dissolved in 4.5 mL of MeOH:HOAc 10:1. The solution was added to the polymer bound reducing agent and the mixture was heated in a microwave oven at 120° C. for 10 minutes. The solution was cooled, filtered, evaporated and re-dissolved in DCM:MeOH (1 mL) and loaded on a Ig Isolute SCX-2 ion exchange column which was washed with 10 mL of MeOH. Elution with 7 mL of 10% Et₃N in MeOH gave the crude title product, which was further purified by flash chromatography (SiO₂, DCM:MeOH 95:5) to yield the title compound (32 mg, 59%).

¹H NMR (400 MHz, CDCl₃) δ 7.67 (d, 1H), 7.20-7.26 (m, 2H), 7.08-7.10 (m, 2H), 7.02 (dd, 1H), 6.84 (s, 1H), 3.90 (s, 3H), 3.89 (m, 2H), 3.66 (s, 2H), 3.52 (dd, 2H), 2.57 (s, 3H), 2.44 (br s, ˜2H), 2.36 (m, 1H), 2.20 (s, 3H), 1.74-1.83 (m, 4H). ¹³C NMR (101 MHz, CDCl₃) δ 158.0, 154.7, 143.8, 143.5, 140.1, 128.67, 128.70, 125.4, 123.6, 122.4, 120.2, 110.2, 103.4, 66.7, 55.8, 54.9, 46.6, 40.7, 36.3, 26.9, 19.7. LC-MS [M+H]⁺ 408.3

### Example 21

### 6-Methoxy-4-methyl-N-[((1R,2S)-2-{[(1-methyl-1H-indol-3-yl)methyl]amino}cyclopentyl)methyl]quinolin-2-amine

### a) Tert-butyl [(1S,2R)-2-(aminomethyl)cyclopentyl]carbamate

tert-butyl [(1S,2R)-2-(azidomethyl)cyclopentyl]carbamate (0.070 g, 0.291 mmol) and 10% Pd on activated carbon (12 mg) in EtOH (5 mL) was thoroughly degassed, and then stirred under an atmosphere of H₂ over night. The mixture was filtered through Celite and concentrated to give 0.062 g (99%) of the title compound.

¹H NMR (400 MHz, MeOH-d₄) δ 3.55-3.49 (m, 1H), 2.72 (dd, J=12.6, 5.7 Hz, 1H), 2.54 (dd, J=12.6, 7.3 Hz, 1H), 1.99-1.25 (m, 16H); ¹³C NMR (101 MHz, MeOH-d₄) δ 158.2, 79.8, 56.4, 50.07, 45.8, 33.6, 29.5, 28.8, 23.2.

### b) Tert-butyl ((1S,2R)-2-{[(6-methoxy-4-methylquinolin-2-yl)amino]methyl}cyclopentyl)carbamate

A mixture of 2-chloro-6-methoxy-4-methylquinoline (0.056 g, 0.27 mmol), tert-butyl [(1S,2R)-2-(aminomethyl)cyclopentyl]carbamate (0.059 g, 0.28 mmol), Cs₂CO₃ (0.220 g, 0.674 mmol), Pd(OAc)₂ (0.005 g, 0.02 mmol), and BINAP (0.013 g, 0.020 mmol) in dioxane (1 mL) under an atmosphere of N₂ was stirred at 80° C. for 21 h. The reaction mixture was cooled to rt, diluted with EtOAc/MeOH 5:1, filtered through a short plug of silica and concentrated. Purification on a 5 g Isolute Flash Si column eluted with a stepwise gradient CH₂Cl₂→heptane→heptane/EtOAc 3:1→2:1→1:1 gave 0.035 g (34%) of the title compound.

¹H NMR (400 MHz, MeOH-d₄) δ 7.55 (d, J=8.9 Hz, 1H), 7.14 (dd, J=8.9, 2.8 Hz, 1H), 7.11 (d, J=2.8 Hz, 1H), 6.60 (s, 1H), 3.84 (s, 3H), 3.64-3.58 (m, 1H), 3.44 (br, 1H), 3.31 (dd, J=13.1, 7.1 Hz, 1H), 2.48 (s, 3H), 2.08-1.33 (m, 16H); LC-MS [M+H]⁺ 386.2.

### c) 6-Methoxy-4-methyl-N-[((1R,2S)-2-{[(1-methyl-1H-indol-3-yl)methyl]amino}cyclopentyl)methyl]quinolin-2-amine

tert-butyl ((1S,2R)-2-{[(6-methoxy-4-methylquinolin-2-yl)amino]methyl}cyclopentyl)-carbamate (0.035 g, 0.091 mmol) was dissolved in CH₂Cl₂ (4 mL) and TFA (1 mL) added. After 2 h, toluene (˜25 mL) was added and the mixture concentrated. The residue and 1-methylindole-3-carbaldehyde (0.021 g, 0.13 mmol) were dissolved in MeOH (1 mL). After 25 h, NEt₃ was added and the mixture allowed to react for an additional 2 days. A second portion of 1-methylindole-3-carbaldehyde (0.035 g, 0.22 mmol) was added and after an additional day, NaBH₄ (0.025 g, 0.66 mmol) was added and the resulting mixture stirred for 15 min. 1M HCl was added and the mixture stirred for an additional 10 min. 1M NaOH (10 mL) was added and the mixture extracted with 2×15 mL EtOAc. The combined organic layers were dried with MgSO₄, filtered and concentrated. The residue was dissolved in CH₂Cl₂ and extracted with 1M HCl. 1M NaOH was added to the aqueous layer until pH14 followed by extraction with EtOAc. The organic layer was dried with MgSO₄, filtered and concentrated to give 0.018 g (46%) of the title compound. ¹H NMR (400 MHz, MeOH-d₄) δ 7.46 (d, J=7.9 Hz, 1H), 7.42 (d, J=9.1 Hz, 1H), 7.26 (d, J=8.3 Hz, 1H), 7.14-7.09 (m, 2H), 7.05, (dd, J=9.1, 2.8 Hz, 1H), 6.97-6.93 (m, 2H), 6.45 (s, 1H), 3.96 (d, J=13.5 Hz, 1H), 3.84 (d, J=13.5 Hz, 1H), 3.84 (s, 3H), 3.64 (s, 3H), 3.43-3.37 (m, 2H), 3.02 (ddd, J=7.0, 7.0, 7.0 Hz, 1H), 2.43 (s, 3H), 2.19-1.37 (m, 7H); ¹³C NMR (101 MHz, MeOH-d₄) δ 157.6, 156.1, 145.3, 144.0, 138.5, 129.0, 128.7, 127.7, 125.2, 122.6, 120.7, 120.0, 119.4, 114.1, 112.7, 110.2, 104.9, 63.9, 56.0, 46.4, 45.9, 43,4, 33.0, 32.7, 30.5, 24.1, 18.9; LC-MS [M+H]⁺ 429.2.

### Example 22

### (1S,3S)- N-(6-Fluoro-4-methylquinolin-2-yl)-N′-[(1-methyl-1H-pyrrolo[2,3-b]pyridin-3-yl)methyl]cyclopentane-1,3-diamine

### a) 1-Methyl-1H-pyrrolo[2,3-b]pyridine 1H-Pyrrolo[2,3-b]pyridine (1.00 g, 8.46 mmol) was dissolved in 10 mL of DMF and cooled on an ice bath. NaH (0.203 g, 8.47 mmol) was added and after 30 min methyl iodide (0.527 mL, 8.47 mmol) was added. The reaction mixture was stirred overnight at room temperature and then poured into 100 mL of water, extracted three times with EtOAc. The combined organic layer was washed with water, dried over Na₂SO₄, filtered and evaporated and a clean product was obtained. Yield: 0.950 g (85%).

¹H NMR (300 MHz, CDCl₃) δ 8.34 (dd, 1H), 7.90 (dd, 1H), 7.18 (d, 1H), 7.05 (dd, 1H), 6.45 (d, 1H), 3.90 (s, 3H).

### b) 1-Methyl-1H-pyrrolo[2,3-b]pyridine-3-carbaldehyde

POCl₃ (1.2 g, 7.9 mmol) was added dropwise with stirring to 10 mL of DMF at 0° C. After stirring 10 min a solution of 1-methyl-1H-pyrrolo[2,3-b]pyridine (0.95 g, 7.2 mmol) in 5 mL of DMF was added during 1 min. The reaction mixture was stirred for 1 h at 0° C. and a further 30 min at 60° C. It was poured into water which was made alkaline with NaHCO₃ (aq) and extracted 3 times with EtOAc. The combined organic layer was washed with water, dried over Na₂SO₄, filtered and evaporated. The crude product (0.85 g, 74%) was sufficiently pure to be used in the subsequent step below.

¹H NMR (300 MHz, CDCl₃) δ 9.97 (s, 1H), 8.55 (m, 1H), 8.44 (m, 1H), 7.84(s, 1H), 7.27 (m, 1H), 3.97 (s, 3H).

### c) (1S,3S)- N-(6-Fluoro-4-methylquinolin-2-yl)-N′-[(1-methyl-1H-pyrrolo[2,3-b]pyridin-3-yl)methyl]cyclopentane-1,3-diamine

(1S,3S)-N-(6-Fluoro-4-methylquinolin-2-yl)cyclopentane-1,3-diamine (76 mg, 0.29 mmol; see Ex 6b) and 1-methyl-1H-pyrrolo[2,3-b]pyridine-3-carbaldehyde (47 mg, 29 mmo; from step b above) were dissolved in 2 mL of methanol and allowed to react overnight. Sodium borohydride (50 mg, 1.3 mmol) was added and the reaction mixture was stirred for 15 min after which 5 mL of 2M HCl was added and after an additional 5 min the mixture was made alkaline by addition of 2M NaOH. The mixture was extracted three times with EtOAc and the combined organic layer was washed with water, dried over Na₂SO₄, filtered and evaporated. The crude product was chromatographed on a pre-packed SiO₂-column (Isolute, 5 g) eluted with DCM:MeOH:Et₃N 100:5:1 to give 78 mg (65%) of the title compound after freeze-drying from dioxane.

¹H NMR (400 MHz, CDCl₃) δ 8.30 (m, 1H), 7.93 (m, 1H), 7.60 (dd, 1H), 7.32 (dd, 1H), 7.24 (m, 1H), 7.07, (s, 1H), 7.01 (dd, 1H), 6.48 (s, 1H), 4.93 (m, 1H), 4.42 (m, 1H), 3.89 (s, 2H), 3.81 (s, 3H), 3.37 (m, 1H), 2.45 (s, 3H), 2.35-2.20 (m, 2H), 2.15-1.95 (m, 2H), 1.80 (m, 1H), 1.50 (m, 1H). ¹³C NMR (101 MHz, CDCl₃) additional signals due to C—F coupling δ 159.9, 157.5, 156.9, 148.9, 145.6, 145.3, 143.8, 129.0, 128.9, 128.0, 127.9, 124.7, 124.6, 120.7, 119.4, 119.1, 116.0, 113.0, 112.7, 108.5, 108.3, 58.3, 52.6, 44.2, 41.6, 33.3, 32.7, 31.9, 19.7. LC-MS [M+H]⁺ 404.2

### Example 23

### (1S,3S)-3-[({3-[(7-Methoxy-4-methylquinolin-2-yl)amino]cyclopentyl}amino)methyl]-1-methyl-1H-indole-6-carbonitrile

### a) 3-Formyl-1H-indole-6-carbonitrile

POCl₃ (0.593 g, 3.87 mmol) was added dropwise with stirring to 5 mL of DMF. After stirring for 10 min 1H-indole-6-carbonitrile (0.500 g, 3.52 mmol) was added in portions. The reaction mixture was stirred for 1 h at ambient temperature and a further 1 h at 40° C. It was then poured into ice water which was made alkaline with NaOH (aq). Thereafter it was heated to 100° C. for 1 min and cooled again with ice and extracted 3 times with EtOAc. The combined organic layer was washed with water, dried over Na₂SO₄, filtered and evaporated. The crude product was recrystallised from water-ethanol to give 0.379 g (63%) of the desired product.

¹H NMR (500 MHz, MeOH-d₄) δ 9.98 (s, 1H), 8.33 (s, 1H), 8.31 (d, 1H), 7.89 (m, 1H), 7.52 (m, 1H).

### b) 3-Formyl-1-methyl-1H-indole-6-carbonitrile

3-Formyl-1H-indole-6-carbonitrile (0.379 g, 2.22 mmol; from step a above) was dissolved in 5 mL of DMF and NaH (80 mg, 3.3 mmol) was added. The mixture was stirred for 5 min after which methyl iodide (0.21 mL, 3.3 mmol) was added. The mixture was allowed to react for 30 min and was then poured into 100 mL of water. The product crystallized and was filtered off, washed with water and dried. Yield: 0.367 g (89%).

¹H NMR (500 MHz, MeOH-d₄) δ 9.92 (s, 1H), 8.31 (d, 1H), 8.29 (s, 1H), 8.02 (s, 1H), 7.56 (dd, 1H), 3.97 (s, 3H).

### c) (1S,3S)-3-[({3-[(7-Methoxy-4-methylquinolin-2-yl)amino]cyclopentyl}amino)methyl]-1-methyl-1H-indole-6-carbonitrile

(1S,3S)-N-(7-Methoxy-4-methylquinolin-2-yl)cyclopentane-1,3-diamine (77 mg, 0.28 mmol) and 3-formyl-1-methyl-1H-indole-6-carbonitrile (52 mg, 0.28 mmol) were dissolved in 2 mL of methanol and allowed to react for two days. Sodium borohydride (54 mg, 1.4 mmol) was added and the reaction mixture was stirred for 30 min after which 5 mL of 2M HCl was added and after an additional 10 min the mixture was made alkaline by addition of 2M NaOH. The mixture was extracted four times with EtOAc and the combined organic layer was washed with water, dried over Na₂SO₄, filtered and evaporated. The crude product was chromatographed on a pre-packed SiO₂-column (Isolute, 5 g) eluted with DCM:MeOH:Et₃N 100:5:1 to give 97 mg (77%) of the title compound after freeze-drying from dioxane.

¹H NMR (400 MHz, CDCl₃) δ 7.67 (d, 1H), 7.59 (d, 1H), 7.55 (m, 1H), 7.28 (dd, 1H), 7.14 (s, 1H), 7.02 (d, 1H), 6.83 (dd, 1H), 6.34 (s, 1H), 4.79 (bd, 1H), 4.41 (m, 1H), 3.89 (s, 2H), 3.85 (s, 3H), 3.70 (s, 3H), 3.35 (m, 1H), 2.47 (s, 3H), 2.30 (m, 1H), 2.08 (m, 1H), 1.99 (m, 1H), 1.82 (m, 1H), 1.55-1.45 (m, 2H). ¹³C NMR (101 MHz, CDCl₃) δ 161.0, 157.4, 150.1, 145.2, 136.1, 131.2, 130.7, 125.0, 122.0, 121.0, 120.1, 118.6, 115.1, 114.4, 113.6, 108.7, 106.0, 104.2, 57.8, 55.5, 51.9, 43.2, 41.1, 33.1, 32.7, 32.1, 19.1. LC-MS [M+H]⁺ 440.2.

### Example 24

### (1S,3S)-N-(6-Fluoro-4-methylquinolin-2-yl)-N′-[(1-methyl-1H-indol-2-yl)methyl]cyclopentane-1,3-diamine

Pol-BH₃CN (289 mg, 1.53 mmol) was suspended (swollen) in 0.8 mL of DCM for 15 min. To this were added (1S,3S)-N-(6-fluoro-4-methylquinolin-2-yl)cyclopentane-1,3-diamine (69 mg, 0.27 mmol; from Example 6b) dissolved in 1.6 mL of DCM:MeOH 1:1, 1-methyl-1H-indole-2-carbaldehyde (39 mg, 0.24 mmol) dissolved in 0.8 mL of DCM, and 80 Pl of HOAc. The mixture was heated in a microwave oven at 100° C. for 10 min. The solution was cooled, filtered, evaporated and dissolved in toluene, evaporated, re-dissolved in toluene and evaporated. The residue was dissolved in 1.3 mL of DCM and aldehyde Wang resin (197 mg, 0.93 mmol) was added and the mixture was stirred at room temperature overnight. The polymer was filtered off and the filtrate was applied to a 1 g Isolute SCX-2 ion exchange column which was washed with 10 mL of MeOH. Elution with 10 mL of 10% Et₃N in MeOH gave the crude title product, which was further purified on a pre-packed SiO₂-column (Isolute, 2 g) eluted with DCM:MeOH:Et₃N 100:5:1. Further purification was done on a 20×250 mm Kromasil C8 column and eluted with a gradient of CH₃CN:0.1 M NH₄OAc 10:90-100:0. The pertinent fractions were combined and the organic solvent evaporated. The product was freeze dried from water. Yield: 44 mg (41%).

¹H NMR (400 MHz, MeOH-d₄) δ 7.57 (dd, 1H), 7.45 (d, 1H), 7.36 (dd, 1H), 7.27 (d, 1H), 7.22 (dd, 1H), 7.10 (t, 1H), 6.98 (t, 1H), 6.58 (s, 1H), 6.39 (s, 1H), 4.47 (m, 1H), 3.92 (s, 2H), 3.68 (s, 3H), 3.41 (m, 1H), 2.40 (s, 3H), 2.25 (m, 1H), 2.13 (m, 1H), 2.02-1.86 (m, 2H), 1.61-1.45 (m, 2H). ¹³C NMR (101 MHz, MeOH-d₄) δ 159.16, 156.72, 144.64, 144.23, 138.04, 137.04, 127.81, 127.16, 127.07, 123.97, 123.88, 121.17, 119.95, 119.13, 117.79, 117.55, 113.53, 108.90, 107.68, 107.46, 100.94, 57.45, 50.86, 43.18, 39.12, 31.55, 30.66, 28.71, 17.51. LC-MS [M+H]⁺ 403.2

### Example 25

### (1S,3S)-N-(6-Fluoro-4-methylquinolin-2-yl)-N-({1-[3-(trifluoromethyl)pyridin-2-yl]-1H-indol-3-yl}methyl)cyclopentane-1,3-diamine

Pol-BH₃CN (252 mg, 1.33 mmol) was suspended (swollen) in 0.8 mL of DCM for 15 min. To this were added (1S,3S)-N-(6-fluoro-4-methylquinolin-2-yl)cyclopentane-1,3-diamine (60 mg, 0.23 mmol; from Ex 6b) dissolved in 1.6 mL of DCM:MeOH 1:1, 1-[3-(trifluoromethyl)pyridin-2-yl]-1H-indole-3-carbaldehyde (60 mg, 0.21 mmol) dissolved in 0.8 mL of DCM, and 80 μl of HOAc. The mixture was heated in a microwave oven at 100° C. for 10 min. The solution was cooled, filtered, evaporated and dissolved in toluene, evaporated, re-dissolved in toluene and evaporated. The residue was dissolved in 1.3 mL of DCM and aldehyde Wang resin (171 mg, 0.81 mmol) was added and the mixture was stirred at room temperature overnight. The polymer was filtered off and the filtrate was applied to a 1 g Isolute SCX-2 ion exchange column which was washed with 10 mL of MeOH. Elution with 10 mL of 10% Et₃N in MeOH gave the crude title product, which was further purified on a 20×250 mm Kromasil C8 column and eluted with a gradient of CH₃CN:0.1M NH₄OAc 10:90-100:0. The pertinent fractions were combined and evaporated. The residue was freeze dried from water and the product was obtained as a partial acetate salt (ca 0.7 eq. HOAc) Yield: 61 mg (41%).

¹H NMR (400 MHz, MeOH-d₄) δ 8.80 (dd, 1H), 8.39 (dd, 1H), 7.77 (m, 1H), 7.70 (m, 1H), 7.61-7.55 (m, 2H), 7.39 (dd, 1H), 7.28-7.13 (m, 4H), 6.60 (s, 1H), 4.54 (m, 1H), 4.31 (s, 2H), 3.70 (m, 1H), 2.44 (d, 3H), 2.36-2.04 (m, 4H), 1.89 (s, 2.1H), 1.82-1.59 (m, 2H). ¹³C NMR(101 MHz, MeOH-d₄) δ 180.16, 160.39, 158.02, 157.74, 154.03, 150.08, 145.75, 145.42, 139.47, 138.99, 130.41, 128.76, 128.46, 128.38, 125.36, 125.22, 125.13, 124.55, 124.11, 122.75, 122.42, 119.72, 118.97, 118.73, 114.82, 112.50, 111.55, 108.88, 108.66, 57.95, 51.92, 42.07, 38.41, 32.37, 30.16, 24.24, 18.67. LC-MS [M+H]⁺ 534.2

### Example 26

### (1S,3S)- N-(6-Fluoro-4-methylquinolin-2-yl)-N′-[(1-methyl-1H-indazol-3-yl)methyl]cyclopentane-1,3-diamine

Pol-BH₃CN (260 mg, 1.38 mmol) was suspended (swollen) in 0.8 mL of DCM for 15 min. To this were added (1S,3S)-N-(6-fluoro-4-methylquinolin-2-yl)cyclopentane-1,3-diamine (62 mg, 0.24 mmol; from Ex 6b) dissolved in 1.6 mL of DCM:MeOH 1:1, 1-methyl-1H-indazole-3-carbaldehyde (34 mg, 0.22 mmol) dissolved in 0.8 mL of DCM, and 80 μl of HOAc. The mixture was heated in a microwave oven at 100° C. for 10 min. The solution was cooled, filtered, evaporated and dissolved in toluene, evaporated, re-dissolved in toluene and evaporated. The residue was dissolved in 1.3 mL of DCM and aldehyde Wang resin (177 mg, 0.84 mmol) was added and the mixture was stirred at room temperature overnight. The polymer was filtered off and the filtrate was applied to a 1 g Isolute SCX-2 ion exchange column which was washed with 10 mL of MeOH. Elution with 10 mL of 10% Et₃N in MeOH gave the crude title product, which was further purified on a pre-packed SiO₂-colurn (Isolute, 5 g) eluted with DCM:MeOH:Et₃N 100:5:1. Further purification was done on a Biotage Horizon silica column eluting with EtOAc.MeOH 95:5->0:100.Yield: 41 mg (42%).

¹H NMR (400 MHz, MeOH-d₄) δ 7.78 (d, 1H), 7.56 (dd, 1H), 7.42 (d, 1H), 7.39-7.31 (m, 2H), 7.22 (m, 1H), 7.10 (m, 1H), 6.57 (s, 1H), 4.64 (m, 1H), 4.07 (s, 2H), 3.96 (s, 3H), 3.35 (m, 1H), 2.41 (s, 3H), 2.24 (m, 1H), 2.10 (m, 1H), 1.98-1.85 (m, 2H), 1.58-1.44 (m, 2H) ¹³C NMR(101 MHz, MeOH-d₄) δ 159.12, 156.76, 144.76, 144.13, 142.70, 141.11, 127.23, 127.15, 126.63, 123.93, 123.85, 122.39, 120.31, 120.26, 117.73, 117.48, 113.49, 109.07, 107.61, 107.38, 57.24, 50.93, 43.32, 39.53, 34.17, 31.63, 31.00, 17.51. LC-MS [M+H]⁺ 404.3

### Example 27

### (1S,3S)-N-(7-Methoxy-4-methylquinolin-2-yl)-N′-({1-[4-(trifluoromethyl)phenyl]-1H-pyrrol-3-yl}methyl)cyclopentane-1,3-diamine

(1S,3S)-N-(7-Methoxy-4-methylquinolin-2-yl)cyclopentane-1,3-diamine (100 mg, 0.37 mmol) and 1-[4-(trifluoromethyl)phenyl]-1H-pyrrole-3-carbaldehyde (88 mg, 0.37 mmol) where dissolved in 10 ml of DCM and NaBH(OAC)₃ (195 mg, 0.92 mmol) was added. The mixture was stirred at rt for 20 h. The reaction was quenched with saturated NH₄Cl, 30 mL DCM was added and the mixture was washed with water. The organic phase was separated and the solvent evaporated. The compound was purified on a pre-packed SiO-column (Isolute, 5 g) eluted with DCM/MeOH (containing 1% NH₄OH aq) 10:1 to give 30 mg (16%) of the title compound.

¹H NMR (400 MHz, MeOH-d₄) δ 7.68-7.55 (m, 5H), 7.25-7.21 (m, 2H), 7.04 (d, 1H), 6.81 (dd, 1H), 6.43 (s, 1H), 6.37 (m, 1H), 4.46 (m, 1H), 3.84 (s, 3H), 3.69 (s, 2H), 3.37 (m, 1H), 2.44 (s, 3H), 2.30-2.09 (m, 2H), 1.93 (m, 2H), 1.61-1.45 (m, 2H) ¹³C NMR (101 MHz, MeOH-d₄ ) δ 161.08, 157.69, 149.58, 144.93, 143.33, 126.78, 126.75, 126.71, 125.78, 124.67, 124.45, 123.08, 119.15, 119.11, 118.17, 117.60, 112.52, 111.86, 109.95, 105.24, 56.76, 54.47, 50.99, 44.09, 39.27, 31.66, 30.73, 17.56 LC-MS [M+H]⁺ 495.09

### Example 28

### 3-[({(1S,3S)-3-[(7-Methoxy-4-methylquinolin-2-yl)amino]cyclopentyl}amino)methyl]-1-methyl-1H-indole-5-carbonitrile

The title compound (40 mg, 30%) was prepared using the procedure described for the preparation of Example 27.

¹H NMR (400 MHz, MeOH-d₄) δ 8.05 (s, 1H), 7.59 (d, 1H), 7.41 (d, 1H), 7.37 (dd, 1H), 7.27 (s, 1H), 7.02 (d, 1H), 6.79 (dd, 1H), 6.41 (s, 1H), 4.44 (m, 1H), 3.89 (s, 2H), 3.83 (s, 3H), 3.75 (s, 3H), 3.34 (m, 1H), 2.42 (s, 3H), 2.29-2.07 (m, 2H), 1.98-1.84 (m, 2H), 1.58-1.45 (m, 2H) ¹³C NMR (101 MHz, MeOH-d₄ ) δ 161.04, 157.66, 149.54, 144.91, 138.83, 130.66, 127.48, 124.65, 124.43, 124.14, 120.66, 118.15, 113.50, 112.50, 110.39, 109.94, 105.20, 101.44, 57.10, 54.49, 51.01, 41.79, 39.43, 31.81, 31.71, 30.88, 17.58 LC-MS [M+H]⁺ 440.1

### Example 29

### (1S,3S)-N-{[5-difluormethoxy-1H-indol-3-yl]methyl}-N′-(7-methoxy-4-methylquinolin-2-yl)cyclopentane-1,3-diamine

### a) 5-{[tert-butyl(dimethyl)silyl]oxy}-1H-indole

1-H-indol-5-ol (1.5 g, 11.3 mmol) was dissolved in DMF (5 mL). Imidazole (1.9 g, 28.2 mmol) and tertbutyldimethylchlorosilane (2.0 g, 13.5 mmol) were added. After stirring at rt for 1.5 h, 20 mL of water was added. The solution was extracted with EtOAc (2×20 mL) and the combined organic phase was washed with water, dried over Na₂SO₄, filtered and evaporated. The resulting oil (3.0 g, containing some DMF) was used in the subsequent step without further purification.

LC-MS [M-H]⁻ 246.05.

### b) 5-{[tert-butyl(dimethyl)silyl]oxy}-1-methyl-1H-indole

5-{[tert-butyl(dimethyl)silyl]oxy}-1H-indole (1.8 g, 7.5 mmol) was dissolved in dry THF (35 mL) and the falsk was placed in an ice bath. NaH (283 mg, 11.2 mmol) was added portionwise until gas evolution had ceased. MeI (2.11 g, 14.9 mmol) was added dropwise. The mixture was stirred for another 1.5 h and was then poured onto crushed ice. The slurry was extracted with EtOAc (3×25 mL) and the combined organic phase was dried over Na₂SO₄, filtered, concentrated and purified or an Isolute 10 g Flash Si pre-packed column eluted with EtOAc/MeOH 9:1 to give 1.42 g (73%) of the title compound.

¹H NMR (400 MHz, CDCl₃) δ 7.16 (d, 1H), 7.08 (d, 1H), 7.01 (d, 1H), 6.81 (dd, 1H), 6.38 (dd, 1H), 3.75 (s, 3H), 1.04 (s, 9H), 0.22 (s, 6H).

### c) 1-methyl-1H-indol-5-ol

5-{[tert-butyl(dimethyl)silyl]oxy}-1-methyl-1H-indole (1.42 g, 5.44 mmol) was dissolved in dry THF (30 mL) and tetra-n-butylammoniumfluoride, trihydrate (1.56 g, 6.00 mmol) was added. After stirring at rt for 2 h, the solution was concentrated, redissolved in a small portion of CH3CN and filtered through a plug of SiO₂. The eluent was concentrated and purified on an Isolute 10 g Flash Si pre-packed column eluted with EtOAc/MeOH 1:1 to give 0.66 g (83%) of the title compound as a solid.

¹H NMR (400 MHz, MeOD) δ 7.14 (d, 1H), 7.01 (d, 1H), 6.91 (d, 1H), 6.71 (dd, 1H), 6.22 (dd, 1H), 3.69(s, 3H).

### d) 5-(difluoromethoxy)-1-methyl-1H-indole

1-methyl-1H-indol-5-ol (0.40 g, 2.72 mmol) was dissolved in iso-PrOH (10 mL) and 30% aq- KOH (10 mL) in a three necked flask equipped with a dry ice condensor. The flask was placed in an oil batch at 70° C. and CHClF₂ (“Freon 22”) was bubbled into the solution for 45 min (at a rate allowing gentle reflux of the Freon). After stirring for an additional 30 min thr oil bath was removed. After the solution had reached rt (2 h), water (100 mL) was added. The solution was extracted with Et₂O and EtOAc and the combined organic phase was washed with 2M aq. NaOH and water. The reulting organic phase was dried over Na₂SO₄, filtered, concentrated and purified is on an Isolute 10 g Flash Si pre-packed column eluted with EtOAc/MeOH 3:1 to give 246 mg (46%) of the title compound.

¹H NMR (400 MHz, MeOD) δ 7.28 (m, 2H), 7.15 (d, 1H), 6.95 (dd, 1H), 6.64 (t, J_{H,F}=76 Hz, 1H), 6.39 (dd, 1H), 3.67 (s, 3H).

### e) 5-(difluoromethoxy)-1-methyl-1H-indole-3-carbaldehyde

Phosphorus oxychloride (0.21 g, 1.37 mmol) was added dropwise to DMF (2.87 g, 37 mmol) under intert atmospere. 5-(difluoromethoxy)-1-methyl-1H-indole (246 mg, 1.25 mmol), dissolved in DMF (0.8 mL) was added dropwise and the resulting mixture was stirred at 35° C. for 35 min. The reaction mixture was oured onto crushed ice. The resulting solution was made alkaline by dropwise addition of a solution of NaOH (240 mg) in H₂O (125 mL). The solution was boiled for 1 minute and was then cooled and extracted with EtOAc.The combined organic phase was dried over Na₂SO₄, filtered and concentrated to give 262 mg (93%) of the title compound as a red solid which was used in the subsequent step without further purification.

¹H NMR (400 MHz, CDCl₃) δ 9.92 (s, 1H), 8.04 (d, 1H), 7.68 (s, 1H), 7.30 (d, 1H), 7.13 (dd, 1H), 6.55 (t, J_{H,F}=74 Hz, 1H), 3.77 (s, 3H).

### e) (1S,3S)-N-{[5-difluormethoxy-1H-indol-3-yl]methyl}-N′-(7-methoxy-4-methylquinolin-2-yl)cyclopentane-1,3-diamine

Using the method described in the preparation of Example 26, the title compound (39 mg, 39%) was obtained as a colourless solid.

¹H NMR (400 MHz, CDCl₃) δ 7.63 (d, 1H), 7.40 (d, 1H), 7.22 (d, 1H), 7.02 (m, 2H), 6.86 (dd, 1H), 6.50 (t, J_{H,F}=75 Hz, 1H), 6.38 (s, 1H), 4.83 (bs, 1H), 4.40 (m, 1H), 3.90 (s, 2H), 3.88 (s, 3H), 3.73 (s, 2H), 3.40 (m, 1H), 2.51 (s, 3H), 2.32 (m, 1H), 2.10 (m, 1H), 2.02 (m, 1H), 1.86 (m, 1H), 1.59-1.47 (m, 2H). LC-MS [M+H]⁺ 481.3.

### Example 30

### (1S,2S,4R,6S)-N-(6-methoxy-4-methylquinolin-2-yl)-N′-(3-thienylmethyl)bicyclo[2.2.1]heptane-2,6-diamine

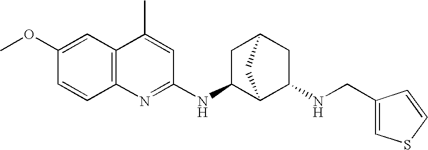

### a) (3R)-3-Hydroxy-1-methylpyrrolidine-2,5-dione

To D(+)-malic acid (14.89 g, 111 mmol) dissolved in hot ethanol (25 mL) was slowly added 2M dimethyl amine in THF (56 mL, 112 mmol). The resulting mixture was concentrated, suspended in o-xylene and heated to reflux using a Dean-Stark head until water evolution ceased (˜3 h). The mixture was concentrated, dissolved in ethyl acetate, filtered through a plug of silica and concentrated. Crystallization from EtOAc/hexane gave 8.94 g (62%) of the title compound.

¹H NMR (400 MHz, MeOH-d₄) δ 4.57 (dd, J=8.4, 4.3 Hz, 1H), 3.04 (dd, J=18.0, 8.4 Hz, 1H), 2.95 (s, 3H), 2.49 (dd, J=18.0, 4.2 Hz, 1H).

### b) (3R)-1-Methyl-2,5-dioxopyrrolidin-3-yl acrylate

To a stirred solution of (3R)-3-hydroxy-1-methylpyrrolidine-2,5-dione (20.35 g, 158 mmol) and triethyl amine (33 mL, 237 mmol) in dry CH₂Cl₂ (250 mL) at 0° C. under an argon atmosphere acryloyl chloride (16 mL, 197 mmol,) was added dropwise. The resulting mixture was stirred for 2 h at 0° C. and 1 h at rt. Water (20 mL) was added and the mixture was stirred for an additional 15 min, washed sequentially with 1M HCl and aqueous NaHCO₃ (sat). The organic layer was dried with MgSO₄, filtered and concentrated. The residue was purified by flash chromatography on silica (gradient heptane/EtOAc 3:1→1:1). Crystallization of the residue from EtOAc/hexane gave 17.44 g (60%) of the title compound.

¹H NMR (400 MHz, CDCl₃) δ 6.51 (dd, J=17.3, 1.2 Hz, 1H), 6.17 (dd, J=17.3, 10.5 Hz, 1H), 5.96 (dd, J=10.5, 1.2 Hz, 1H), 5.52 (dd, J=8.7, 4.6 Hz, 1H), 3.21 (dd, J=18.3, 8.7 Hz, 1H), 3.06 (s, 3H), 2.71 (dd, J=18.3, 4.6 Hz, 1H); ¹³C NMR (101 MHz, CDCl₃) δ 173.6, 173.4, 165.1, 133.5, 127.0, 67.8, 36.0, 25.3.

### c) (3R)-1-Methyl-2,5-dioxopyrrolidin-3-yl(1S,2S,4S)-bicyclo[2.2.1]hept-5-ene-2-carboxylate

To a stirred solution of (3R)-1-methyl-2,5-dioxopyrrolidin-3-yl acrylate (17.33 g, 94.6 mmol) in CH₂Cl₂/hexane 4:1 (200 mL) at −35° C. under an argon atmosphere was added 1M TiCl₄ in hexane (10 mL). After 30 min when the mixture had attained −25° C., freshly distilled cyclopentadiene (8.20 g, 124 mmol) was added and the mixture stirred for an additional 1 h 30 min after which the mixture had attained −10° C. The reaction was quenched by addition af finely powdered Na₂CO₃×10 H₂O (10 g). After warming to rt the mixture was filtered and concentrated. Recrystallization from EtOAc/hexane gave 19.54 g (83%) of the title compound.

¹H NMR (400 MHz, CDCl₃) δ 6.23 (dd, J=5.6, 3.0 Hz, 1H), 5.93 (dd, J=5.6, 2.8 Hz, 1H), 5.34 (dd, J=8.7, 4.6 Hz, 1H), 3.24 (bs, 1H), 3.13, (dd, J=18.3, 8.7 Hz, 1H), 3.08-3.04 (m, 1H), 3.05 (s, 3H), 2.94 (bs, 1H), 2.60 (dd, J=18.3, 4.6 Hz, 1H), 1.94 (ddd, J=11.8, 9.3, 3.7 Hz, 1H), 1.48-1.42 (m, 2H), 1.30 (d, J=8.3 Hz, 1H).

### d) (1S,2S,4S)-Bicyclo[2.2.1]hept-5-ene-2-carboxylic acid

To a stirred solution of (3R)-1-methyl-2,5-dioxopyrrolidin-3-yl (1S,2S,4S)-bicyclo[2.2.1]hept-5-ene-2-carboxylate (5.50 g, 22.1 mmol) in THF (70 mL) was added a solution of LiOH (2.35 g, 98.1 mmol) in H₂O (55 mL). After 63 h, the mixture was concentrated until ˜50 mL remained. The aqueous solution was acidified with conc HCl and extracted with 2×75 mL n-pentane/CH₂Cl₂ 98:2. The combined organic layers were dried with MgSO₄, filtered and concentrated to give 3.01 g (99%) of the title compound.

¹H NMR (400 MHz, CDCl₃) δ 6.20 (dd, J=5.6, 3.0 Hz, 1H), 5.99 (dd, J=5.6, 2.8 Hz, 1H), 3.23 (bs, 1H), 2.99, (dt, J=9.3, 4.0 Hz, 1H), 2.91 (bs, 1H), 1.91 (ddd, J=11.8, 9.4, 3.6 Hz, 1H), 1.46-1.37 (m, 2H), 1.28 (d, J=8.3 Hz, 1H); ¹³C NMR (101 MHz, CDCl₃) δ 181.0, 138.1, 132.6, 49.9, 45.9, 43.4, 42.8, 29.3.

### e) (3S,3aR,5S,6S,6aS)-6-Iodohexahydro-2H-3,5-methanocyclopenta[b]furan-2-one

To a stirred solution of (1S,2S,4S)-bicyclo[2.2.1]hept-5-ene-2-carboxylic acid (3.00 g, 21.7 mmol) and Na₂CO₃×10H₂O (34.9 g, 122 mmol) in H₂O (200 mL) was added droppwise over 15 min a solution of I₂ (8.84 g, 34.8 mmol) and KI (17.38 g, 104.7 mmol) in H₂O (100 mL). The resulting mixture was stirred for 30 min. CH₂Cl₂ (100 ML) and 1M Na₂S₂O₃ (100 mL) was added and the mixture stirred until the brown colour disappeared. The phases were separated and the organic layer washed with aqueous Na₂CO₃ (sat), dried with MgSO₄, filtered and concentrated to give 4.44 g (77%) of the title compound.

¹H NMR (400 MHz, CDCl₃) δ 5.05 (d, J=5.0 Hz, 1H), 3.83 (d, J=2.6 Hz, 1H), 3.14 (tdd, J=4.8, 1.4, 2.6 Hz, 1H), 2.65 (d, J=1.0 Hz, 1H), 2.50 (dd, J=11.3, 4.6 Hz, 1H), 2.30 (dd, J=11.5, 1.8 Hz, 1H), 2.01 (ddd, J=13.5, 11.3, 4.0 Hz, 1H), 1.81-1.75 (mn, 2H); ¹³C NMR (101 MHz, CDCl₃) δ 179.5, 89.1, 47.0, 46.8, 37.7, 37.6, 34.7, 30.3.

### f) (1R,2S,4S)-6-Oxobicyclo[2.2.1]heptane-2-carboxylic acid

A solution of KOH (4.35 g, 56.1 mmol) in MeOH (7.5 mL) and H₂O (30 mL) was added to (3S,3aR,5S,6S,6aS)-6-iodohexahydro-2H-3,5-methanocyclopenta[b]furan-2-one (6.60 g, 25.0 mmol). The resulting mixure was stirred for 4 days, acidified with conc HCl and extracted with 4×30 mL EtOAc. The combined organic layers were washed with brine (15 mL), dried with MgSO₄, filtered and concentrated to give 4.04 g (purity 70% by ¹H NMR, 73% yield) of the title compound,

¹H NMR (400 MHz, CDCl₃) δ 2.98 (br, 1H), 2.89 (d, J=4.2 Hz, 1H), 2.68 (bs, 1H), 2.11-2.02 (m, 2H), 1.98 (dd, J=16.7, 3.6 Hz, 1H), 1.86-1.78 (m, 2H), 1.69 (bd, J=10.7 Hz, 1H).

### g) (1R,2S,4S,6S)-6-{Benzyl[(benzyloxy)carbonyl]amino}bicyclo[2.2.1]heptane-2-carboxylic acid

(1R,2S,4S)-6-oxobicyclo[2.2.1 ]heptane-2-carboxylic acid (4.00 g, 154 mmol) and benzylamine (3.00 g, 28.0 mmol) were dissolved in MeOH (100 mL). After 22 h, the mixture was concentrated and dissolved in CH₂Cl₂ (150 mL). Sodium triacetoxyborohydride (9.6 g, 45.3 mmol) was added and the resulting mixture stirred for 23 h. A second portion of sodium triacetoxyborohydride (3.1 g. 14.6 mmol) was added and after an additional 2 h, the mixture was concentrated. 1M NaOH (100 mL) was added and the resulting mixture stirred for 1 h, neutralized with conc HCl and the volume reduced to ˜100 mL. Dioxane (100 mL), Na₂CO₃×10H₂O (40.0 g, 140 mmol) and N-(benzyloxycarbolnyloxy)succinimide (9.7 g, 38.9 mmol) was added, and the resulting mixture stirred for 3 days. A second portion of N-(benzyloxycarbolnyloxy)succinimide (2.0 g, 8.0 mmol) was added and the mixture stirred over night. H₂O (500 mL) was added followed by acidification with conc HCl and extraction with 2×100 mL EtOAc. The combined organic layers were washed with brine (25 mL), dried with MgSO₄, filtered and concentrated. Purification by Flash chromatography (heptane/EtOAc 3:1) gave 2.2 g of an impure product. The impure product was dissolved in EtOAc (10 mL) and loaded on a 15 g Isolute NH₂-ion exchange column, washed with EtOAc (50 mL), MeOH (50 mL), and eluted with MeOH/HOAc 10:1 (50 mL). The product fraction was diluted with toluene, concentrated and crystallized from EtOAc/hexane to give 1.41 g (20%) of the title compound.

¹H NMR (400 MHz, MeOH-d₄) δ 7.31-7.09 (m,10H), 5.12 (d, J=12.6 Hz, 1H), 5.08 (d, J=12.6 Hz, 1H), 4.69 (d, J=17.3 Hz, 1H), 4.49 (d, J=17.3 Hz, 1H), 4.14 (dd, J=7.6, 5.3 Hz, 1H), 2.73 (ddd, J=11.3, 5.5, 4.4 Hz, 1H), 2.59 (bd, J=3.6 Hz, 1H), 2.25 (bs, 1H), 1.85 (ddd, J=12.9, 8.3, 2.2 Hz, 1H), 1.70-1.50 (m, 4H), 1.36 (d, J=10.1 Hz, 1H); ¹³C NMR (101 MHz, MeOH-d₄) δ 176.2, 157.3, 139.4, 136.9, 128.3, 128.2, 127.8, 127.6, 126.6, 126.0, 67.1, 55.7, 46.8, 45.2, 44.3, 39.9, 38.2, 36.5, 30.2; LC-MS [M−H]⁻ 378.1.

### h) Benzyl[(1R,2S,4S,6S)-6-aminobicyclo[2.2.1]hept-2-yl]benzylcarbamate

(1R,2S,4S,6S)-6-{benzyl[(benzyloxy)carbonyl]amino}bicyclo[2.2.1]heptane-2-carboxylic acid (1.39 g, 3.66 mmol) was dissolved in CH₂Cl₂/toluene and concentrated. The resulting syrup and NEt₃ (0.66 mL, 4.74 mmol) was dissolved in dry acetone (6 mL). To the stirred mixture at 0° C. was added ethyl chloroformate. After 30 min sodium azide (0.351 g, 5.40 mmol) dissolved in H₂O (2 mL) was added. The resulting mixture was stirred for 2 h at 0° C. followed by addition of H₂O (100 mL) and extraction with 3×25 mL toluene. The combined organic layers were dried with MgSO₄, filtered and the filter cake washed with toluene (25 mL). The filtrate was stirred and heated to 100° C. for 30 min, concentrated and dissolved in THF (100 mL). 1M HCl (10 mL) was added and the mixture left for 3 days. H₂O (100 mL) was added and the mixture neutralized with 1M NaOH (10 mL). The THF was removed by reducing the volume to ˜100 mnL on a rotary evaporator. 1M NaOH (10 mL) was added and the mixture extracted with 2×50 mL EtOAc. To the combined organic layers MeOH (3 mL) was added and the the resulting solution washed with 0.02M NaOH (50 mL). After addition of abs EtOH (50 mL), the organic layer was concentrated and purified on an Isolute 10 g Flash Si pre-packed column eluted with EtOAc/MeOH 5:1 containing 1% NEt₃, to give 0.931 g of the title compound (73%).

¹H NMR (400 MHz, CDCl₃) δ 7.32-7.14 (m, 10H), 5.16 (s, 2H), 4.68-4.64 (m, 2H), 4.55 (d, J=16.7 Hz, 1H), 3.20 (ddd, J=10.2, 5.0, 5.0 Hz, 1H), 2.14 (bs, 1H), 2.06 (d, J=3.0 Hz, 1H), 1.92-1.80 (m, 2H), 1.63-1.57 (m, 1H), 1.48 (d, J=9.9 Hz, 1H), 1.31-1.20 (m, 3H), 0.55 (ddd, J=12.6, 5.1, 2.6 Hz, 1H); ¹³C NMR (101 MHz, CDCl₃) δ 157.0, 139.7, 136.9, 128.5, 128.4, 127.9, 126.7, 126.2, 67.2, 52.4, 51.7, 47.8, 47.3, 39.9, 37.7, 37.3, 36.9; LC-MS [M+H]⁺ 351.2.

### i) Benzyl benzyl{(1R,2S,4S,6S)-6-[(6-methoxy-4-methylquinolin-2-yl)amino]bicyclo[2.2.1]hept-2-yl}carbamate

A mixture of 2-chloro-6-methoxy-4-methylquinoline (0.050 g, 0.24 mmol), benzyl [(1R,2S,4S,6S) 6-aminobicyclo[2.2.1]hept-2-yl]benzylcarbamate (0.067 g, 0.19 mmol), Cs₂CO₃ (0.153 g, 0.47 mmol), Pd(OAc)₂ (0.005 g, 0.022 mmol,), and BINAP (0.015 g, 0.024 mmol) in toluene (1.5 mL) under an atmosphere of N₂ was stirred at 90° C. for 40 h. The reaction mixture was cooled to rt, diluted with EtOAc/MeOH 10:1, filtered through a short plug of silica and concentrated. Purification on a 5 g Isolute Flash Si columnn eluted with a stepwise gradient CH₂Cl₂→heptane→heptane/EtOAc 3:1→1:1→EtOAc gave 0.049 g (49%) of the title compound.

¹H NMR (400 MHz, CDCl₃) δ 7.54 (d, J=9.1 Hz, 1H), 7.26-6.99 (m, 12H), 6.34 (bs, 1H), 5.10 (d J=12.4 Hz, 1H), 5.02 (d, J=12.4 Hz, 1H), 4.76 (bs, 1H), 4.64 (d, J=16.9 Hz, 1H), 4.52-4.45 (m, 2H), 4.07 (bs, 1H), 3.90 (s, 3H), 2.79 (d, J=3.4 Hz, 1H), 2.46 (s, 3H), 2.29-2.17 (m, 2H), 2.0-1.82 (m, 1H), 1.69-1.62 (m, 1H), 1.57 (d, J=10.2 Hz, 1H), 1.47 (d, J=10.2 Hz, 1H), 0.83-0.87 (m, 1H); ¹³C NMR (101 MHz, CDCl₃) δ 157.1, 155.6, 154.9, 143.7, 143.6, 139.4, 137.0, 128.6, 128.5, 128.0, 126.7, 126.3, 124.3, 120.0, 112.2, 103.7, 67.3, 55.8, 53.1, 52.8, 47.7, 45.1, 40.7, 37.1, 37.0, 36.5, 19.1; LC-MS [M+H]⁺ 522.2.

### j) (1S,2S,4R,6S)-N-(6-methoxy-4-methylquinolin-2-yl)bicyclo[2.2.1]heptane-2,6-diamine

benzyl benzyl {(1R,2S,4S,6S)-6-[(6-methoxy-4-methylquinolin-2-yl)amino]bicyclo[2.2.1]hept-2-yl}carbamate (0.025 g, 0.048 mmol) and 10% Pd on activated carbon (20 mg) in EtOH (5 mL) was thoroughly degassed, and then stirred under an atmosphere of H₂. After 40 h, the mixture was filtered through Celite and concentrated to give 0.012 g (84%) of the title compound.

¹H NMR (400 MHz, MeOH-d₄) δ 7.56 (d, J=9.1 Hz, 1H), 7.18-7.14 (m, 2H), 6.67 (s, 1H), 4.26 (ddd, J=11.3, 4.5, 4.5 Hz, 1H), 3.87 (s, 3H), 3.33 (m, 1H), 2.65 (d, J=3.6 Hz, 1H), 2.51 (s, 3H), 2.31 (bs, 1H), 2.18-2.10 (m, 1H), 1.91 (ddd, J=12.8, 8.0, 2.0 Hz, 1H), 1.68 (d, J=10.2 Hz, 1H), 1.49 (d, J=10.2 Hz, 1H), 1.33-1.27 (m, 1H), 0.93 (ddd, J=12.7, 4.7, 2.9 Hz, 1H); LC-MS [M+H]⁺ 298.2.

### k) (1S,2S,4R,6S)-N-(6-methoxy-4-methylquinolin-2-yl)-N′-(3-thienylmethyl)bicyclo[2.2.1]heptane-2,6-diamine

A solution of thiophene-3-carbaldehyde (0.005 g, 0.040 mmol) in MeOH (0.3 mL) was added to (1S,2S,4R,6S)-N-(7-methoxy-4-methylquinolin-2-yl)bicyclo[2.2.1]heptane-2,6-diamine (0.012 g, 0.040 mmol) and Pol-BH₃CN (0.110 g) in 0.9 mL CH₂Cl₂. HOAc (0.03 mL) was added and the resulting slurry subjected to microwave single node heating 100° C. for 10 min. LC/MS indicated ˜50% conversion of (1S,2S,4R,6S)-N-(7-methoxy-4-methylquinolin-2-yl)bicyclo[2.2.1 ]heptane-2,6-diamine into product. A second portion of thiophene-3-carbaldehyde (0.002 mg, 0.020 mmol) in MeOH (0.15 mL) and Pol-BH₃CN (0.050 g) was added and the resulting slurry subjected to microwave single node heating 100° C. for an additional 10 min. The resin was filtered off and washed with portions (1-2 mL) of CH₂Cl₂ and MeOH. The filtrate was concentrated and purified by C8-HPLC (0.1M ammonium acetate buffer: 5% CH₃CN→100% CH₃CN) to give, after freeze-drying, 0.006 g (33%) of the title compound as an acetate salt.

¹H NMR (400 MHz, MeOH-d₄) δ 7.64 (d, J=9.5 Hz, 1H), 7.28 (dd, J=4.9, 2.9 Hz, 1H), 7.25-7.21 (m, 2H), 7.13 (d, J=1.8 Hz, 1H), 6.96 (dd, J=5.0, 1.2 Hz, 1H), 6.65 (s, 1H), 4.30 (ddd, J=11.1, 4.6, 4.6 Hz, 1H), 4.09 (d, J=13.7 Hz, 1H), 4.03 (d, J=13.7 Hz, 1H), 3.89 (s, 3H), 3.40 (dd, J=7.9, 4.1 Hz, 1H), 3.19 (d, J=3.6 Hz, 1H), 2.57 (s, 3H), 2.43 (bs, 1H), 2.22-2.14 (m, 1H), 2.00-1.94 (m, 1H), 1.93 (acetate), 1.78 (d, J=11.1 Hz, 1H), 1.66-1.60 (m, 2H), 1.04 (ddd, J=12.9, 4.8, 2.8 Hz, 1H); LC-MS [M+H]⁺ 394.2.

### Example 31

### (1R,2S,4S,6S)-N-(6-methoxy-4-methylquinolin-2-yl)-N′-(3-thienylmethyl)bicyclo[2.2.1]heptane-2,6-diamine

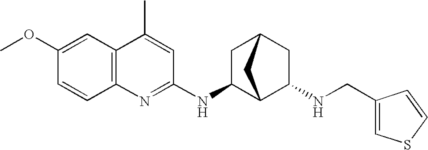

### a) Dibenzyl (2S,6S)-bicyclo[2.2.1]heptane-2,6-diylbiscarbamate

Benzyl [(1R,2S,4S,6S)-6-aminobicyclo[2.2.1]hept-2-yl]benzylcarbamate (0.056 g, 0.16 mmol) and 10% Pd on activated carbon (25 mg) in EtOH (10 mL) was thoroughly degassed, and then stirred under an atmosphere of H₂. After 18 h, the mixture was filtered through Celite, and the filter cake rinsed with 20 mL EtOH. To the filtrate was added benzyl chloroformate (0.091 mL, 0.64 mmol) and N,N-diisopropylethylamine (0.111 mL, 0.64 mmol). After 1 h, the mixture was concentrated, dissolved in EtOAc (25 mL), washed sequentially with 1M HCl and H₂O, dried with MgSO₄, filtered and concentrated. Purification on a 2 g Isolute Flash Si column eluted with a stepwise gradient CH₂Cl₂→heptane→heptane/EtOAc 3:1→1:1 gave 0.039 g (62%) of the title compound.

¹H NMR (400 MHz, CDCl₃) δ 7.37-7.27 (m, 10H), 5.16-5.00 (m, 5H), 4.80-4.70 (br, 1H), 4.00-3.75 (br, 2H), 2.42 (bs, 1H), 2.26 (bs, 1H), 2.11-2.01 (m, 1H), 1.92-1.84 (m, 1H), 1.43-1.23 (m, 3H), 0.72 (bd, J=11.7 Hz, 1H); ¹³C NMR (101 MHz, CDCl₃) δ 156.5, 155.8, 136.7, 128.8, 128.7 128.4, 66.9, 51.1, 47.8, 47.0, 41.7, 36.6, 36.4, 35.6, 32.1.

### b) (1R,2S,4S,6S)-N-(6-methoxy-4-methylquinolin-2-yl)bicyclo[2.2.1]heptane-2,6-diamine

A mixture of 2-chloro-6-methoxy-4-methylquinoline (0.23 g, 0.11 mmol), dibenzyl (2S,6S)-bicyclo[2.2.1]heptane-2,6-diylbiscarbamate (0.039 g, 0.099 mmol), Cs₂CO₃ (0.090 g, 0.28 mmol), Pd(OAc)₂ (0.003 g, 0.013 mmol), and BINAP (0.009 g, 0.014 mmol) in toluene (1 mL) under an atmosphere of N₂ was stirred at 70° C. for 40 h. The reaction mixture was cooled to rt, diluted with EtOAc/MeOH 10:1, filtered through a short plug of silica and concentrated. Purification on a 2 g Isolute Flash Si column eluted with a stepwise gradient CH₂Cl₂→heptane→heptane/EtOAc 3:1→1:1 gave 0.029 g (purity ˜60% by ¹H-NMR) of dibenzyl {(1R,2S,4S,6S)-6-[(6-methoxy-4-methylquinolin-2-yl) amino]bicyclo[2.2.1]hept-2,6-diyl}biscarbamate along with unreacted dibenzyl (2S,6S)-bicyclo[2.2.1]heptane-2,6-diylbiscarbamate. The product mixture and 10% Pd on activated carbon (14 mg) in EtOH (5 mL) was thoroughly degassed, and then stirred under an atmosphere of H₂. After 1 h, the mixture was filtered through Celite and concentrated. Purification on a 0.5 g Isolute Flash Si column eluted with EtOAc/MeOH containing 1% NEt₃ gave 0.009 g (31%) of the title compound.

¹H NMR (400 MHz, MeOH-d₄) δ 7.62 (d, J=8.9 Hz, 1H), 7.20-7.15 (m, 2H), 6.63 (s, 1H), 4.24 (dd, J=8.1 Hz, 1H), 3.87 (s, 3H), 3.58 (ddd, J=11.1, 4.4, 4.4 Hz, 1H), 2.59 (d, J=4.0 Hz, 1H), 2.51 (s, 3H), 2.40 (bs, 1H), 2.16-2.02 (m, 2H), 1.76 (d, J=10.8 Hz, 1H), 1.61-1.54 (m, 1H), 1.47 (d, J=10.8 Hz, 1H), 1.10 (ddd, J=13.3, 3.6, 3.6 Hz, 1H); LC-MS [M+H]⁺ 298.2.

### c) (1R,2S,4S,6S)-N-(6-methoxy-4-methylquinolin-2-yl)-N′-(3-thienylmethyl)bicyclo[2.2.1]heptane-2,6-diamine

A solution of thiophene-3-carbaldehyde (0.003 g, 0.030 mmol) in MeOH (0.3 mL) was added to (1R,2S,4S,6S)-N-(6-methoxy-4-methylquinolin-2-yl)bicyclo[2.2.1]heptane-2,6-diamine (0.009 g, 0.030 mmol) and Pol-BH₃CN (0.120 g) in 0.9 mnL CH₂Cl₂. HOAc (0.03 mL) was added and the resulting slurry subjected to microwave single node heating 100° C. for 10 min. The resin was filtered off and washed with portions (1-2 mL) of CH₂Cl₂ and MeOH. The filtrate was concentrated and purified by C8-HPLC (0.1M ammonium acetate buffer:5% CH₃CN→100% CH₃CN) to give, after freeze-drying, 0.008 g (58%) of the title compound as an acetate salt.

¹H NMR (400 MHz, MeOH-d₄) δ 7.66 (d, J=2.0 Hz, 1H), 7.56 (dd, J=4.9, 2.9 Hz, 1H), 7.42 (d, J=9.1 Hz, 1H), 7.33 (dd, J=4.9, 1.1 Hz, 1H), 7.17 (d, J=2.8 Hz, 1H), 7.07 (dd, J=9.1, 2.8 Hz, 1H), 6.65 (s, 1H), 5.10 (d, J=13.3 Hz, 1H), 4.59 (d, J=13.3 Hz, 1H), 4.26 (dd, J=8.2, 2.9 Hz, 1H), 3.86 (s, 3H), 3.54 (ddd, J=11.0, 4.3, 4.3 Hz, 1H), 2.80 (d, J=3.6 Hz, 1H), 2.52 (s, 3H), 2.41 (bs, 1H), 2.14-2.04 (m, 2H), 1.93 (acetate), 1.74 (d, J=10.7 Hz, 1H), 1.62-1.56 (m, 1H), 1.42 (d, J=10.7 Hz, 1H), 1.14 (ddd, J=13.3, 3.6, 3.6 Hz, 1H); LC-MS [M+H]⁺ 394.2.

### Example 32

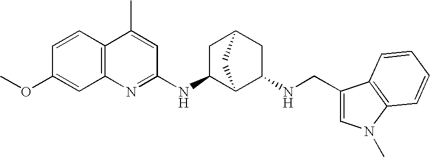

### (1S,2S,4R,6S)-N-(7-methoxy-4-methylquinolin-2-yl)-N′-[(1-methyl-1H-indol-3-yl)methyl]bicyclo[2.2.1]heptane-2,6-diamine

### a) Benzyl benzyl{(1R,2S,4S,6S)-6-[(7-methoxy-4-methylquinolin-2-yl)amino]bicyclo[2.2.1]hept-2-yl}carbamate

A mixture of 2-chloro-7-methoxy-4-methylquinoline (0.220 g, 1.06 mmol), benzyl [(1R,2S,4S,6S)-6-aminobicyclo[2.2.1]hept-2-yl]benzylcarbamate (0.270 g, 0.770 mmol), NaO^{t}Bu (0.121 g, 1.26 mmol), Pd(OAc)₂ (0.011 g, 0.049 mmol), and BINAP (0.029 g, 0.046 mmol) in toluene (2.5 mL) under an atmosphere of N₂ was subjected to microwave single node heating 140° C. for 15 min. The reaction mixture was cooled to rt, diluted with EtOAc/MeOH 10:1, filtered through a short plug of silica and concentrated. Purification on a 10 g Isolute Flash Si column eluted with a stepwise gradient CH₂Cl₂→heptane→heptane/EtOAc 3:1→1:1 gave 0.277 g (69%) of the title compound.

¹H NMR (400 MHz, CDCl₃) δ 7.63 (d, J=8.9 Hz, 1H), 7.27-7.00 (m, 10H), 6.99 (d, J=2.4 Hz, 1H), 6.89 (dd, J=9.0, 2.5 Hz, 1H), 6.20 (br, 1H), 5.11 (d, J=12.5 Hz, 1H), 5.03 (d, J=12.5 Hz, 1H), 4.91 (br, 1H), 4.66 (d, J=16.7 Hz, 1H), 4.53-4.45 (m, 2H), 4.12-4.04 (m, 1H), 3.88 (s, 3H), 2.82 (d, J=3.0 Hz, 1H), 2.44 (s, 3H), 2.31-2.16 (m, 2H), 1.99-1.89 (m, 1H), 1.71-1.64 (m, 1H), 1.58 (d, J=10.2 Hz, 1H), 1.48 (d, J=10.2 Hz, 1H), 0.82 (bd, J=13 Hz, 1H); ¹³C NMR(101 MHz, CDCl₃) δ 160.8, 157.4, 157.1, 150.1, 144.7, 139.5, 137.0, 128.6, 128.5, 128.0, 126.7, 126.2, 124.8, 118.7, 113.7, 109.6, 106.5, 67.3, 55.5, 53.2, 52.7, 47.7, 45.1, 40.8, 37.1, 37.0, 36.5, 18.9.

### b) (1S,2S,4R,6S)-N-(7-methoxy-4-methylquinolin-2-yl)bicyclo[2.2.1]heptane-2,6-diamine

benzyl benzyl {(1R,2S,4S,6S)-6-[(7-methoxy-4-methylquinolin-2-yl)amino]bicyclo[2.2.1 ]hept-2-yl}carbamate (0.204 g, 0.391 mmol) and 10% Pd on activated carbon (100 mg) in EtOH (25 mL) was thoroughly degassed, and then stirred under an atmosphere of H₂. After 64 h, the mixture was filtered through Celite and concentrated. Purification on a 5 g Isolute Flash Si column eluted with EtOAc/MeOH, containing 1% NEt₃ gave 0.074 g (64%) of the title compound.

¹H NMR (400 MHz, MeOH-d₄) δ 7.65 (d, J=8.9 Hz, 1H), 7.06 (d, J=2.6 Hz, 1H), 6.83 (dd, J=8.9, 2.6 Hz, 1H), 6.50 (s, 1H), 4.26 (ddd, J=11.1, 4.6, 4.6 Hz, 1H), 3.86 (s, 3H), 3.25 (dd, J=7.8, 3.7 Hz, 1H), 2.58 (d, J=3.4 Hz, 1H), 2.47 (s, 3H), 2.29 (bs, 1H), 2.16-2.08 (m, 1H), 1.89 (dd, J=12.7, 8.0, 2.2 Hz, 1H), 1.67 (bd, J=10.3 Hz, 1H), 1.45 (bd, J=10.3 Hz, 1H), 1.25 (ddd, J=12.8, 7.3, 3.7 Hz, 1H), 0.91 (ddd, J=12.7, 4.7, 2.9 Hz, 1H); LC-MS [M+H]⁺ 298.3.

### c) (1S,2S,4R,6S)-N-(7-methoxy-4-methylquinolin-2-yl)-N′-[(1-methyl-1H-indol-3- yl)methyl]bicyclo[2.2.1]heptane-2,6-diamine

(1S,2S,4R,6S)-N-(7-methoxy-4-methylquinolin-2-yl)bicyclo[2.2.1]heptane-2,6-diamine (0.074 g, 0.249 mmol) and 1-methylindole-3-carbaldehyde (0.040 g, 0.249 mmol) was dissolved in MeOH (1 mL). After 16 h, Pol-BH₃CN (0.150 g) was added and the resulting slurry stirred for 2 days. The resin was filtered off and washed with portions (1-2 mL) of CH₂Cl₂ and MeOH. The filtrate was concentrated and purified on a 5 g Isolute Flash Si column eluted with EtOAc/MeOH, containing 1% NEt₃ to give 0.071 g (64%) of the title compound.

¹H NMR (400 MHz, MeOH-d₄) δ 7.68 (d, J=9.1 Hz, 1H), 7.39 (d, J=8.1 Hz, 1H), 7.20 (d, J=8.3 Hz, 1H), 7.10-7.05 (m, 2H), 6.91-6.84 (m, 2H), 6.64 (s, 1H), 6.43 (s, 1H), 4.22 (ddd, J=11.1, 4.5, 4.5 Hz, 1H), 3.87 (s, 3H), 3.82 (d, J=13.7, Hz, 1H), 3.69 (d, J=13.7 Hz, 1H), 3.47 (s, 3H), 3.15 (dd, J=7.4, 4.4 Hz, 1H), 2.97 (d, J=3.4 Hz, 1H), 2.91 (d, J=1.0 Hz, 3H), 2.26 (bs, 1H), 2.16-2.07 (m, 1H), 1.78 (ddd, J=12.4, 8.0, 2.1 Hz, 1H), 1.67 (d, J=10.3 Hz, 1H), 1.44 (d, J=10.3 Hz, 1H), 1.31 (ddd, J=12.7, 7.2, 3.9 Hz, 1H), 0.91 (ddd, J=12.7, 4.8, 2.8 Hz, 1H); ¹³C NMR (101 MHz, MeOH-d₄) δ 161.2, 158.2, 149.7, 144.7, 137.3, 127.8, 127.5, 124.7, 121.2, 118.7, 118.2, 118.1, 112.6, 111.6, 110.2, 108.9, 105.4, 54.5, 51.3, 48.2, 43.7, 41.4, 40.0, 36.6, 35.9, 34.7, 31.3, 17.6; LC-MS [M+H]⁺ 441.3.

### Example 33

### 6-Methoxy-4-methyl-N-[(1S,2R)-2-({[(1-methyl-1H-indol-3-yl)methyl]amino}methyl)cyclopentyl]quinolin-2-amine

### a) Tert-butyl [(1S,2S)-2-(hydroxymethyl)cyclopentyl]carbamate

To (1S,2S)-2-[(tert-butoxycarbonyl)amino]cyclopentanecarboxylic acid (0.995 g, 4.34 mmol) in THF (5 mL) at 0° C. under N₂ was added sequentially triethyl amine (0.67 mL, 4.8 mmol) and ethyl chloroformate (0.46 mL, 4.8 mmol). The resulting mixture was stirred for 30 min at 0° C. The temperature allowed to attain rt, the precipitate filtered off and the filtrate added droppwise to NaBH₄ (0.247 g, 6.54 mmol) in H₂O at 0° C. The resulting mixture was stirred for 30 min at 0° C., and 2 h at rt. The reaction mixture was acidified to pH1 by addition of 2M HCl and extracted with 10×5 mL EtOAc. The combined organic layers were washed with aqueous NaHCO₃ (sat), dried with MgSO₄, filtered and concentrated to give 0.607 g (65%) of the title compound.

¹H NMR (400 MHz, MeOH-d₄) δ 3.61-3.50 (m, 2H), 3.44 (dd, J=10.8, 6.5 Hz, 1H), 1.95-1.33 (m, 16H).

### b) {(1S,2S)-2-[(Tert-butoxycarbonyl)amino]cyclopentyl}methyl methanesulfonate

To a stirred solution of tert-butyl [(1S,2S)-2-(hydroxymethyl)cyclopentyl]carbamate (0.591 g, 2.74 mmol) in CH₂Cl₂ (10 mL) at 0° C. was added sequentially NEt₃ (0.76 mL, 5.5 mmol) and methanesulfonyl chloride (0.32 mL, 4.1 mmol). The resulting mixture was allowed to attain rt and stirred for 16 h. 1M HCl (10 mL) was added, the phases separated and the aqueous layer extracted with CH₂Cl₂ (10 mL). The combined organic layers were dried with MgSO₄, filtered and concentrated. The residue was dissolved in MeOH and crystallized at −78° C. to give 0.611 g (76%) of the title compound.

¹H NMR (400 MHz, MeOH-d₄) δ 4.28 (dd, J=9.8, 5.1 Hz, 1H), 4.16 (dd, J=9.8, 6.7 Hz, 1H), 3.67 (ddd, J=7.9, 7.9, 7.9 Hz, 1H), 3.07 (s, 3H), 2.13-1.40 (m, 16H); ¹³C NMR (101 MHz, MeOH-d₄) δ 156.9, 78.8, 72.0, 53.9, 45.6, 35.9, 32.4, 27.7, 27.2, 22.0.

### c) Tert-butyl [(1S,2R)-2-(azidomethyl)cyclopentyl]carbamate

A stirred mixture {(1S,2S)-2-[(tert-butoxycarbonyl)amino]cyclopentyl}methyl methanesulfonate (0.420 g, 1.43 mmol) and NaN₃ (0.249 g, 3.83 mmol) in DMF (2 mL) was heated to 160° C. for 15 min. The mixture was cooled to rt, H₂O (25 mL) and EtOAc (15 mL) was added. The phases were separated and the aqueous layer extracted with EtOAc (15 mL). The combined organic layers were dried MgSO₄, filtered and concentrated to give 0.309 g (90%) of the title compound.

¹H NMR (400 MHz, MeOH-d₄) δ 6.66 (br, 1H), 3.65-3.57 (m, 1H), 3.45 (dd, J=12.3, 4.8 Hz, 1H 3.29 (d, J=12.3, 6.8 Hz, 1H), 2.02-1.88 (m, 3H), 1.77-1.59 (m, 2H), 1.52-1.37 (m, 11H); ¹³C NMR (101 MHz, MeOH-d₄) δ 157.0, 78.7, 55.0, 54.2, 45.9, 32.3, 28.2, 27.6, 21.9.

### d) N-[(1S,2R)-2-(azidomethyl)cyclopentyl]-6-methoxy-4-methylquinolin-2-amine

A mixture of 2-chloro-6-methoxy-4-methylquinoline (0.040 g, 0.193 mmol), tert-butyl [(1S,2R)-2 (azidomethyl)cyclopentyl]carbamate (0.040 g, 0.166 mmol), NaO^{t}Bu (0.029 g, 0.302 mmol), Pd(OAc)₂ (0.002 g, 0.008 mmol), and BINAP (0.005 g, 0.008 mmol) in toluene (1 mL) under an atmosphere of N₂ was subjected to microwave single node heating 140° C. for 20 min. The reaction mixture was cooled to rt, diluted with EtOAc/MeOH 10:1, filtered through a short plug of silica and concentrated. Purification on a 5 g Isolute Flash Si column eluted with a stepwise gradient CH₂Cl₂→heptane→heptane/EtOAc 3:1 gave 0.033 g of tert-butyl [(1S,2R)-2-(azidomethyl)cyclopentyl](6-methoxy-4-methylquinolin-2-yl)carbamate containing ˜30% N-[(1S,2R)-2-(azidomethyl)cyclopentyl]-6-methoxy-4-methylquinolin-2-amine. The product was dissolved in CH₂Cl₂ (4 mL) and TFA (1 mL) added. After 2 h, 1M NaOH (20 mL) and CH₂Cl₂ (2(mL) was added. The phases were separated, and the organic layer dried with MgSO₄, filtered and concentrated. Purification on a 5 g Isolute Flash Si column eluted with heptane/EtOAc gave 0.011 g (21%) of the title compound.

¹H NMR (400 MHz, CDCl₃) δ 7.59 (d, J=9.1 Hz, 1H), 7.20 (dd, J=9.1, 2.8 Hz, 1H), 7.08 (d, J=2.8 Hz, 1H), 6.50 (s, 1H), 4.45 (d, J=7.7 Hz, 1H), 4.16-4.09 (m, 1H), 3.89 (s, 3H), 3.61 (dd, J=12.3, 4.9 Hz, 1H), 3.39 (d, J=12.3, 7.2 Hz, 1H), 2.53 (s, 3H), 2.25-2.17 (m, 1H), 2.06-1.94 (m, 2H), 1.79-1.71 (m, 2H), 1.55-1.45 (m, 2H); LC-MS [M+H]⁺ 312.3.

### e) N-[(1S,2R)-2-(aminomethyl)cyclopentyl]-6-methoxy-4-methylquinolin-2-amine

N-[(1S,2R)-2-(azidomethyl)cyclopentyl]-6-methoxy-4-methylquinolin-2-amine (0.011 g, 0.035 mmol) and 10% Pd on activated carbon (7 mg) in EtOH (3 mL) was thoroughly degassed, and ther stirred under an atmosphere of H₂ over night. The mixture was filtered through Celite and concentrated to give 0.010 g (99%) of the title compound.

¹H NMR (400 MHz, MeOH-d₄) δ 7.49 (d, J=9.5 Hz, 1H), 7.18-7.14 (m, 2H), 6.61 (s, 1H), 4.11 (ddd, J=6.7, 6.7, 6.7 Hz, 1H), 3.87 (s, 3H), 2.85 (dd, J=12.7, 7.2 Hz, 1H), 2.74 (dd, J=12.7, 5.4 Hz, 1H), 2.50 (s, 3H), 2.18-2.09 (m, 1H), 2.03-1.89 (m, 2H), 1.85-1.58 (m, 3H), 1.46-1.37 (m, 1H); LC-MS [M+H]⁺ 286.2.

### f) 6-Methoxy-4-methyl-N-[(1S,2R)-2-({[(1-methyl-1H-indol-3-yl)methyl]amino}methyl)cyclopentyl]quinolin-2-amine

N-[(1S,2R)-2-(aminomethyl)cyclopentyl]-6-methoxy-4-methylquinolin-2-amine (0.010 g, 0.035 mmol) and 1-methylindole-3-carbaldehyde (0.025 g, 0.157 mmol) was dissolved in MeOH (1 ML). After 3 days, NaBH4 (0.010 g, 0.264 mmol) was added and the resulting mixture stirred for 15 min 1M HCl (2 mL) was added and the mixture stirred for an additional 10 min. 1M NaOH (5 mL) and H₂O was added, and the mixture extracted with 2×15 mL EtOAc. The combined organic layers were dried with MgSO₄, filtered and concentrated. The residue was purified by C8-HPLC (0.1M ammonium acetate buffer, 40% CH₃CN, isocratic followed by gradient→100% CH₃CN) to give 0.006 g (40%) of the title compound.

¹H NMR (400 MHz, MeOH-d₄) δ 7.46 (d, J=7.8 Hz, 1H), 7.25 (d, J=8.3 Hz, 1H), 7.19 (d, J=9.1 Hz, 1H), 7.12 (ddd, J=7.5, 7.5, 0.8 Hz, 1H), 7.09 (d, J=2.8 Hz, 1H), 6.95-6.89 (m, 2H), 6.82 (s, 1H), 6.53 (s, 1H), 4.15 (ddd, J=6.7, 6.7, 6.7 Hz, 1H), 4.00 (d, J=13.4 Hz, 1H), 3.95 (d, J=13.4 Hz, 1H), 3.87 (s, 3H), 3.59 (s, 3H), 2.90 (dd, J=11.9, 9.1 Hz, 1H), 2.80 (dd, J=11.9, 4.9 Hz, 1H), 2.46 (s, 3H), 2.16-2.01 (m, 3H), 1.85-1.58 (m, 3H), 1.42-1.31 (m, 1H); LC-MS [M+H]⁺ 429.2.

### Example 34

### (1S,3S)-N-(7-Methoxy-4-methylquinolin-2-yl)-N′-[(1-methyl-1H-pyrrolo[3,2-h]quinolin-3-yl)methyl]cyclopentane-1,3-diamine

### a) 1H-Pyrrolo[3,2-h]quinoline-3-carbaldehyde

POCl₃ (1.00 g, 6.5 mmol) was added dropwise with stirring to 10 mL of DMF at 0° C. After stirring 10 min 1H-pyrrolo[3,2-h]quinoline (1.00 g, 5.9 mmol) was added. The reaction mixture was stirred for 2 h at room temperature and a further 1 hmin at 70° C. and 2 h at 90° C. It was poured into water which was made alkaline with NaOH (aq) and the mixture was heated to reflux for 1 min. After cooling it was extracted 3 times with EtOAc. The combined organic layer was washed with water, dried over Na₂SO₄, filtered and evaporated. The crude product was flash chromatographed on silica gel with DCM/MeOH 95/5 to yield 0.61 g (52%) of the desired compound.

¹H NMR (300 MHz, CDCl₃) δ 10.18 (s, 1H), 8.91 (dd, 1H), 8.48 (d, 1H), 8.34 (dd, 1H), 7.97 (s, 1H), 7.69 (d, 1H), 7.52 (dd, 1H).

### b) 1-Methyl-1H-pyrrolo[3,2-h]quinoline-3-carbaldehyde

1H-Pyrrolo[3,2-h]quinoline-3-carbaldehyde (0.61 g, 3.1 mmol; from step a above) was dissolved in 10 mL of DMF and NaH (112 mg, 4.7 mmol) was added. The mixture was stirred for 5 min after which methyl iodide (0.19 mL, 3.1 mmol) was added. The mixture was allowed to react for 30 min and was then poured into 200 mL of water. The product crystallized and was filtered off, washed with water and dried. Yield: 0.56 g (86%).

¹H NMR (300 MHz, CDCl₃) δ 10.11 (s, 1H), 8.89 (dd, 1H), 8.44 (d, 1H), 8.25 (dd, 1H), 7.76 (s, 1H), 7.63 (d, 1H), 7.42 (dd, 1H), 4.60 (s, 3H).

### c) (1S,3S)-N-(7-Methoxy-4-methylquinolin-2-yl)-N′-[(1-methyl-1H-pyrrolo[3,2-h]quinolin-3-yl)methyl]cyclopentane-1,3-diamine

(1S,3S)-N-(7-Methoxy-4-methylquinolin-2-yl)cyclopentane-1,3-diamine (85 mg, 0.31 mmol) and 1-methyl-1H-pyrrolo[3,2-h]quinoline-3-carbaldehyde (66 mg, 0.31 mmol; from step b above) were dissolved in 2 mL of methanol and allowed to react overnight. Sodium borohydride (59 mg, 1.6 mmol) was added and the reaction mixture was stirred for 30 min after which 5 mL of 2M HCl was added and after an additional 10 min the mixture was made alkaline by addition of 2M NaOH. The mixture was extracted four times with EtOAc and the combined organic layer was washed with water, dried over Na₂SO₄, filtered and evaporated. The crude product was chromatographed on a pre-packed SiO₂-column (Isolute, 5 g) eluted with DCM:MeOH:Et₃N 100:5:1 to give 92 mg (63%) of the title compound after freeze-drying from dioxane.

¹H NMR (500 MHz, CDCl₃) δ 8.85 (dd, 1H), 8.18 (dd, 1H), 7.81 (d, 1H), 7.65 (d, 1H), 7.42 (d, 1H), 7.33 (dd, 1H), 7.09 (d, 1H), 6.90 (dd, 1H), 6.40 (s, 1H), 4.83 (m, 1H), 4.51 (s, 3H), 4.46 (m, 1H), 4.04 (s, 2H), 3.91 (s, 3H), 3.47 (m, 1H), 2.52 (bd, 3H), 2.37 (m, 1H), 2.20-2.05 (m, 2H), 1.89 (m, 1H), 1.65-1.50 (m, 2H). ¹³C NMR (75 MHz, CDCl₃) δ 161.0, 157.3, 150.0, 147.6, 145.3, 140.4, 136.1, 130.1, 128.5, 126.9, 125.7, 125.0, 119.9, 119.4, 118.9, 118.6, 114.9, 113.7, 108.5, 105.9, 57.8, 55.6, 52.2, 43.5, 41.3, 37.9, 32.9, 32.2, 19.3. LC-MS [M+H]⁺ 466.2.

### Example 35

### (1S,3S)-N-(6-Fluoro-4-methylquinolin-2-yl)-N′-[(1-methyl-1H-pyrrolo[2,3-c]pyridin-3-yl)methyl]cyclopentane-1,3-diamine

### a) 1-Methyl-1H-pyrrolo[2,3-c]pyridine

1H-Pyrrolo[2,3-c]pyridine (0.500 g, 4.23 mmol) was dissolved in dry THF and cooled on an ice bath. Sodium hydride (152 mg, 6.35 mmol) was added and the reaction was stirred for 5 min. Methyl iodide (0.39 mL, 6.3 mmol) was added and the stirring continued for 30 min. The mixture was poured into water and extracted three times with diethyl ether. The combined ethereal layer was washed with water, dried over Na₂SO₄ and evaporated. The product was used as such in the following step. Yield: 180 mg (32%).

¹H NMR (300 MHz, CDCl₃) δ 8.76 (s, 1H), 8.24 (d, 1H), 7.50 (m, 1H), 7.16 (d, 1H), 6.48 (m, 1H), 3.89 (s, 3H).

### b) 1-Methyl-1H-pyrrolo[2,3-c]pyridine-3-carbaldehyde

1-Methyl-1H-pyrrolo[2,3-c]pyridine (0.180 g, 1.36 mmol) and hexamethylenetaetramine (0.38 g, 2.7 mmol) were dissolved in 5 mL of TFA and heated to 80° C. for 4 h with stirring. TFA was evaporated and the residue was partitioned between water and EtOAc. The mixture was made alkaline by addition of 2M NaOH. The aqueous layer was extracted with EtOAc twice and the combined organic layer was washed with water, dried over Na₂SO₄ and evaporated. The crude product was flash chromatographed on silica with DCM/MeOH 95/5. Yield: 99 mg (45%).

¹H NMR (300 MHz, CDCl₃) δ 10.02 (s, 1H), 8.81 (bd, 1H), 8.47 (d, 1H), 8.12 (m, 1H), 7.78 (s, 1H), 3.97 (s, 3H).

### c) (1S,3S)-N-(6-Fluoro-4-methylquinolin-2-yl)-N′-[(1-methyl-1H-pyrrolo[2,3-c]pyridin-3-yl) methyl]cyclopentane-1,3-diamine

(1S,3S)-N-(6-Fluoro-4-methylquinolin-2-yl)cyclopentane-1,3-diamine (75 mg, 0.29 mmol) and 1-methyl-1H-pyrrolo[2,3-c]pyridine-3-carbaldehyde (44 mg, 0.28 mmol) were dissolved in 5 mL of DCM and the reaction stirred for four days. The solvent was evaporated and the residue dissolved in 10 mL of methanol and sodium borohydride (100 mg, 2.6 mmol) was added. The reaction was stirred for 30 min after which the solvent was evaporated. The residue was partitioned between water and EtAOc. The aqueous layer was extracted with EtOAc twice and the combined organic layer was washed with water, dried over Na₂SO₄ and evaporated. The crude product was chromatographed on a 2 g Isolute Si column with DCM/MeOH/TEA 100/5/1. Yield: 68 mg (57%).

¹H NMR (400 MHz, CDCl₃) δ 8.72 (s, 1H), 8.25 (d, 1H), 7.62 (dd, 1H), 7.53 (m, 1H), 7.35 (dd, 1H), 7.26 (m, 1H), 7.13 (s, 1H), 6.52 (s, 1H), 4.78 (bd, 1H), 4.46 (m, 1H), 3.92 (s, 2H), 3.82 (s, 3H), 3.39 (m, 1H), 2.48 (s, 3H), 2.32 (m, 1H), 2.15-2.00 (m, 2H), 1.84 (m, 1H), 1.60-1.45 (m, 2H). ¹³C NMR (100 MHz, CDCl₃) δ 159.9, 157.6, 157.0, 145.9, 145.22, 145.18, 139.2, 135.0, 133.6, 133.0, 131.7, 129.24, 129.16, 124.8, 124.7, 119.4, 119.1, 114.40, 114.35, 112.7, 108.6, 108.3, 58.4, 52.6, 43.8, 41.7, 33.8, 33.4, 32.7, 19.8. LC-MS [M+H]⁺ 404.2.

### Example 36

### (1S,3S)-N-(7-Methoxy-4-methylquinolin-2-yl)-N′-[(1-methyl-1H-pyrrolo[3,2-b]pyridin-3-yl)methyl]cyclopentane-1,3-diamine

### a) 1H-Pyrrolo[3,2-b]pyridine-3-carbaldehyde

The compound was made according to Example 35, step b, above from 1H-pyrrolo[3,2-b]pyridine (V. A. Azimov, L. N. Yakhontov; Chem. Heterocycl. Compounds Engl. Transl. 1971, 13, 1145) (110 mg, 0.93 mmol) and hexamethylenetetramine (261 mg, 1.86 mmol) in 4 mL of TFA. Yield: 63 mg (46%). The crude material was used without purification in the next step.

¹H NMR (300 MHz, CD₃OD) δ 10.14 (s, 1H), 8.49 (m, 1H), 8.33 (s, 1H), 7.94 (m, 1H), 7.32 (m, 1H).

### b) 1-Methyl-1H-pyrrolo[3,2-b]pyridine-3-carbaldehyde

1H-Pyrrolo[3,2-b]pyridine-3-carbaldehyde (63 mg, 0.43 mmol) was dissolved in 2 mL of DMF and sodium hydride (15 mg, 0.64 mmol) was added. After stirring for 5 min methyl iodide (61 mg, 0.43 mmol) was added and the reaction mixture was stirred for 1.5 h. The mixture was poured into water and extracted three times with EtOAc. The combined organic layer was washed with water, dried over Na₂SO₄ and evaporated. The crude product was chromatographed on a 1 g Isolute Si column with DCM/MeOH 95/5. Yield: 30 mg (43%).

¹H NMR (300 MHz, CDCl₃) δ 10.24 (s, 1H), 8.60 (bd, 1H), 7.89 (s, 1H), 7.66 (d, 1H), 7.22 (m, 1H), 3.86 (s, 3H).

### c) (1S,3S)-N-(7-Methoxy-4-methylquinolin-2-yl)-N′-[(1-methyl-1H-pyrrolo[3,2-b]pyridin-3-yl)methyl]cyclopentane-1,3-diamine

(1S,3S)-N-(7-Methoxy-4-methylquinolin-2-yl)cyclopentane-1,3-diamine (51 mg, 0.19 mmol) and 1-methyl-1H-pyrrolo[3,2-b]pyridine-3-carbaldehyde (30 mg, 0.19 mmol) were dissolved in 2 mL of methanol and allowed to react overnight. Sodium borohydride (35 mg, 0.94 mmol) was added and the mixture was stirred for 30 min. The reaction was stopped by addition of 2M HCl and after 5 min stirring the mixture was made alkaline with 2M NaOH and poured into water. The aqueous layer was extracted three times with EtOAc and the combined organic layer was washed with water, dried over Na₂SO₄ and evaporated. The crude product was chromatographed on a 300×50 mm Kromasil C8 column 100 Å 10μ and eluted with a gradient of CH₃CN:0.1M NH₄OAc 20:80-100:0. The pertinent fractions were combined and the organic solvent evaporated. The residue was made alkaline by NaOH (aq) and extracted three times with EtOAc. The combined organic layer was washed with water, dried over Na₂SO₄, filtered and evaporated. Yield: 46 mg (59%).

¹H NMR (300 MHz, CDCl₃) δ 8.43 (m, 1H), 7.61 (d, 1H), 7.55 (m, 1H), 7.20 (s, 1H), 7.10 (dd, 1H), 7.01 (d, 1H), 6.84 (dd, 1H), 6.38 (s, 1H), 4.83 (bd, 1H), 4.37 (m, 1H), 4.04 (s, 2H), 3.87 (s, 3H), 3.74 (s, 3H), 3.43 (m, 1H), 2.50 (s, 3H), 2.45-2.25 (m, 2H), 2.20-1.95 (m, 2H), 1.86 (m, 1H), 1.65-1.45 (m, 2H). ¹³C NMR (100 MHz, CDCl₃) δ 161.0, 157.4, 150.0, 145.6, 145.4, 142.7, 131.0, 130.3, 124.9, 118.5, 116.6, 116.5, 114.3, 113.6, 108.3, 105.8, 57.5, 55.5, 52.2, 42.7, 41.1, 33.0, 32.8, 31.9, 19.1. LC-MS [M+H]⁺ 416.2.

### Example 37

### (1S,3S)-N-(6-Fluoro-4-methylquinolin-2-yl)-N′-(imidazo[1,2-a]pyridin-3-ylmethyl) cyclopentane-1,3-diamine

### a) Imidazo[1,2-a]pyridine-3-carbaldehyde

Imidazo[1,2-a]pyridine (0.500 g, 4.23 mmol) was dissolved in 1 mL of DMF and phosphorus oxychloride (0.71 g, 4.6 mmol) was added dropwise and the mixture was stirred and checked on LC-MS. After 1 hthe mixture was poured into water and made alkaline with 1M NaOH. The mixture was extracted three times with EtOAc and the combined organic layer was washed with water, dried over Na₂SO₄, filtered and evaporated. The crude product was flash chromatographed on silica gel with DCM:MeOH 99:1-96:4. Yield: 83 mg (13%).

1H NMR (300 MHz, CDCl₃) δ 9.95 (s, 1H), 9.50 (m, 1H), 8.32 (s, 1H), 7.80 (m, 1H), 7.56 (m, 1H), 7.13 (m, 1H).

### b) (1S,3S)-N-(6-Fluoro-4-methylquinolin-2-yl)-N′-(imidazo[1,2-a]pyridin-3-ylmethyl) cyclopentane-1,3-diamine

(1S,3S)-N-(6-Fluoro-4-methylquinolin-2-yl)cyclopentane-1,3-diamine (76 mg, 0.29 mmol) and imidazo[1,2-a]pyridine-3-carbaldehyde (43 mg, 0.29 mmol) were dissolved in 2 mL of DCM and allowed to react for 4 h. Sodium triacetoxyborohydride (112 mg, 0.53 mmol) was added and the mixture was stirred overnight. Much of the imine was left according to LC-MS. Sodium borohydride (50 mg, 1.3 mmol) was added and stirring was continued for 30 min. The mixture was acidified with 2M HCl and after 5 min the mixture was poured into water which was made alkaline with 2M NaOH. The mixture was extracted three times with EtOAc and the combined organic layer was washed with water, dried over Na₂SO₄, filtered and evaporated. The crude product was chromatographed on a 2 g Isolute Si column with DCM/MeOH/TEA 100/5/1. Yield: 91 mg (77%).

¹H NMR (400 MHz, CDCl₃) δ 8.29 (m, 1H), 7.60 (dd, 1H), 7.56 (m, 1H), 7.46 (s, 1H), 7.31 (dd, 1H), 7.23 (m, 1H), 7.13 (m, 1H), 6.77 (m, 1H), 6.46 (s, 1H), 4.85 (bd, 1H), 4.44 (m, 1H), 4.03 (s, 2H), 3.29 (m, 1H), 2.44 (bd, 3H), 2.27 (m, 1H), 2.10-1.95 (m, 2H), 1.77 (m, 1H), 1.55-1.35 (m, 2H). ¹³C NMR (100 MHz, CDCl₃) δ 159.9, 157.5, 156.9, 147.0, 145.7, 145.12, 145.08, 133.1, 129.2, 129.1, 125.7, 124.9, 124.8, 124.7, 123.3, 119.3, 119.1, 118.5, 113.0, 112.8, 108.6, 108.4, 58.6, 52.3, 43.0, 41.5, 33.1, 32.7, 19.7. LC-MS [M+H]⁺ 390.2.

### Example 38

### (1S,3S)-N-{[5-(Benzyloxy)-1-methyl-1H-indol-3-yl]methyl}-N′-(7-methoxy-4-methylquinolin-2-yl) cyclopentane-1,3-diamine

### a) 5-(Benzyloxy)-1H-indole-3-carbaldehyde

Phosphorus oxychloride (1.5 g, 9.9 mmol) was added dropwise with stirring to 15 mL of DMF cooled on an ice bath. The cooling bath was removed and the mixture was allowed to react for 15 min. (Benzyloxy)-1H-indole (CAS No 1215-59-4) (2.00 g, 8.96 mmol) was added and the mixture heated to 50-60° C. for 1.5 h. It was then poured into ice water and made alkaline with 2M NaOH. The mixture was refluxed for 2 min and after cooling filtered to give a powder which was washed with water and dried. Yield: 1.88 g (84%).

¹H NMR (400 MHz, DMSO-d₆) δ 9.88 (s, 1H), 8.20 (s, 1H), 7.68 (d, 1H), 7.50-7.25 (m, 6H), 6.96 (dd, 1H), 5.11 (s, 2H), 4.02 (s, 1H).

### b) 5-(Benzyloxy)-1-methyl-1H-indole-3-carbaldehyde

5-(Benzyloxy)-1H-indole-3-carbaldehyde (0.50 g, 2.0 mmol) was suspended in 5 mL of DMF and stirred. Sodium hydride (72 mg, 3.0 mmol) was added and when the gas evolution had subsided after 5 min methyl iodide (0.42 g, 3.0 mmol) was added and allowed to react for 30 min. The mixture was poured into water and extracted 4 times with chloroform. The combined organic layer was washed with water, dried over Na₂SO₄, filtered and evaporated. The crude product was pure. Yield: 0.50 g (95%).

¹H NMR (500 MHz, CDCl₃) δ 9.94 (s, 1H), 7.92 (bd, 1H), 7.60 (s, 1H), 7.52-7.48 (m, 2H), 7.43-7.38 (m, 2H), 7.33 (m, 1H), 7.24 (d, 1H), 7.06 (dd, 1H), 5.16 (s, 2H), 3.83 (s, 3H).

### c) (1S,3S)-N-{[5-(Benzyloxy)-1-methyl-1H-indol-3-yl]methyl}-N′-(7-methoxy-4-methylquinolin-2-yl) cyclopentane-1,3-diamine

(1S,3S)-N-(7-Methoxy-4-methylquinolin-2-yl)cyclopentane-1,3-diamine (75 mg, 0.28 mmol) and 5-(benzyloxy)-1-methyl-1H-indole-3-carbaldehyde (73 mg, 0.28 mmol) were dissolved in 2 mL of methanol and stirred for 40 h. Sodium borohydride (52 mg, 1.38 mmol) was added and stirring was continued for 30 min. The mixture was acidified with 2M HCl and after 5 min the mixture was poured into water which was made alkaline with 2M NaOH. The mixture was extracted three times with EtOAc and the combined organic layer was washed with water, dried over Na₂SO₄, filtered and evaporated. The crude product was chromatographed on a 5 g Isolute Si column with DCM/MeOH/TEA 100/5/1. Yield: 110 mg (75%).

¹H NMR (500 MHz, CDCl₃) δ 7.64 (d, 1H), 7.50-7.45 (m, 2H), 7.40-7.35 (m, 2H), 7.32 (m, 1H), 7.22-7.18 (m, 2H), 7.07 (m, 1H), 7.02-6.98 (m, 2H), 6.89 (m, 1H), 6.40 (s, 1H), 5.14 (s, 2H), 4.80 (bd, 1H), 4.44, (m, 1H), 3.92 (s, 2H), 3.90 (s, 3H), 3.71 (s, 3H), 3.43 (m, 1H), 2.52 (s, 3H), 2.34 (m, 1H), 2.11 (m, 1H), 2.05 (m, 1H), 1.86 (m, 1H), 1.60-1.50 (m, 2H). ¹³C NMR (100 MHz, CDCl₃) δ 160.7, 157.1, 152.9, 149.7, 145.0, 137.6, 132.6, 128.3, 127.9, 127.6, 127.4, 124.6, 118.2, 113.3, 112.7, 112.4, 109.9, 108.1, 105.6, 102.5, 70.9, 57.1, 55.2, 51.7, 43.1, 40.7, 32.6, 32.4, 31.6, 18.8. LC-MS [M+H]⁺ 521.3.

The title compounds of Examples 39-42 were prepared from (1S,3S)-N-(7-methoxy-4-methylquinolin-2-yl)cyclohexane-1,3-diamine by reductive amination alkylation with the appropriate aldehyde;

A mixture of (1S,3S)-N-(7-methoxy-4-methylquinolin-2-yl)cyclohexane-1,3-diamine (0.16 mmol), the aldehyde (0.16 mmol) and Pol-BH₃CN (0.100 g) in MeOH/CH₂Cl₂ 3:1 (1.5 mL), containing HOAc (0.03 mL) was stirred for 3 days. The resin was filtered off and washed with portions (1-2 mL) of MeOH. The filtrate was concentrated and purified by C8-HPLC (0.1M ammonium acetate buffer: 5% CH₃CN→100% CH₃CN). Fractions containing the product were concentrated, dissolved in EtOAc, washed with 1M NaOH, dried with MgSO₄, filtered and concentrated to give the title compounds in 39-56% yield.

### Example 39

### (1S,3S)-N-(7-methoxy-4-methylquinolin-2-yl)-N′-[3-(trifluoromethoxy)benzyl]cyclohexane-1,3-diamine

¹H NMR (500 400 MHz, CDCl₃ MeOH-d₄) δ 7.61 (d, J=8.9 Hz, 1H), 7.26-7.22 (m, 3H), 7.08-7.04 (m, 1H), 7.01 (d, J=2.4 Hz, 1H), 6.80 (dd, J=8.9, 2.4 Hz, 1H), 6.45 (s, 1H), 4.35-4.28 (m, 1H), 3.81 (s, 3H), 3.80 (d, J=13.7 Hz, 1H), 3.76 (d, J=13.7 Hz, 1H), 2.89-2.81 (m, 1H), 2.44 (s, 3H), 2.1-1.93 (mn, 1H), 1.80-1.55 (m, 6H), 1.44-1.34 (m, 1H) ¹³C NMR (100 MHz, CDCl₃) LC-MS [M+H]⁺ 460.1

### Example 40

### (1S,3S)-N-(2,1,3-benzothiadiazol-4-ylmethyl)-N′-(7-methoxy-4-methylquinolin-2-yl) cyclohexane-1,3-diamine

¹H NMR (500 400 MHz, CDCl₃ MeOH-d₄) δ 7.76 (d, J=8.5 Hz, 1H), 7.60 (d, J=9.1 Hz, 1H), 7.40-7.33 (m, 2H), 6.96 (d, J=2.2 Hz, 1H), 6.79 (dd, J=9.0, 2.3 Hz, 1H), 6.35 (s, 1H), 4.32-4.26 (m, 1H), 4.22 (s, 2H), 3.81 (s, 3H), 2.84-2.76 (m, 1H), 2.42 (s, 3H), 2.12-2.04 (m, 1H), 1.82-1.54 (m, 6H), 1.45-1.35 (m, 1H). ¹³C NMR (100 101 MHz, CDCl₃ MeOH-d₄) δ 162.2, 158.4, 156.3, 155.4, 150.7, 146.0, 133.8, 130.6, 129.2, 125.8, 121.0, 119.4, 113.7, 111.2, 106.4, 55.7, 52.3, 47.9, 46.7, 36.9, 32.6, 32.1, 20.8, 18.8 LC-MS [M+H]⁺ 434.1

### Example 41

### (1S,3S)-N-[(1,3-dimethyl-1H-pyrazol-4-yl)methyl]-N′-(7-methoxy-4-methylquinolin-2-yl)cyclohexane-1,3-diamine

¹H NMR (500 400 MHz, CDCl₃ MeOH-d₄) δ 7.62 (d, J=9.1 Hz, 1H), 7.28 (s, 1H), 7.01 (d, J=2.4 Hz, 1H), 6.80 (dd J=8.9, 2.4 Hz, 1H), 6.48 (s, 1H), 4.35-4.28 (m, 1H), 3.83 (s, 3H), 3.63 (s, 3H), 3.57 (s, 2H), 2.90-2.84 (m, 1H), 2.45 (s, 3H), 2.11 (s, 3H), 2.10-2.02 (m, 1H), 1.84-1.56 (m, 6H), 1.42-1.32 (m, 1H). ¹³C NMR (100 101 MHz, CDCl₃ MeOH-d₄) δ 162.3, 158.6, 150.8, 148.1, 146.1, 132.2, 125.8, 119.4, 118.1, 113.7, 111.3, 106.4, 55.7, 52.3, 46.7, 40.3, 38.3, 37.1, 32.4, 32.2, 20.9, 18.8, 11.3 LC-MS [M+H]⁺ 394.2

### Example 42

### (1S,3S)-N-(2-bromo-4-methoxybenzyl)-N′-(7-methoxy-4-methylquinolin-2-yl)cyclohexane-1,3-diamine

¹H NMR (500 400 MHz, CDCl₃ MeOH-d₄) δ 7.61 (d, J=8.9 Hz, 1H), 7.32 (d, J=8.9 Hz, 1H), 7.00 (d, J=2.4 Hz, 1H), 6.97 (d, J=3.1 Hz, 1H), 6.79 (dd, J=9.1, 2.4 Hz, 1H), 6.65 (dd, J=8.7, 3.0 Hz, 1H), 6.45 (s, 1H), 4.35-4.28 (m, 1H), 3.82 (s, 3H), 3.82 (d, J=13.9 Hz, 1H), 3.78 (d, J=13.9 Hz, 1H), 3.63 (s, 3H), 2.92-2.84 (m, 1H), 2.44 (s, 3H), 2.02-1.92 (m, 1H), 1.82-1.55 (m, 6H), 1.47-1.35 (m, 1H). ¹³C NMR (100 101 MHz, CDCl₃ MeOH-d₄) δ 162.2, 160.7, 158.5, 150.7, 146.1, 142.0, 134.3, 125.8, 119.4, 117.1, 115.6, 115.0, 113.7, 111.2, 106.4, 55.8, 55.7, 52.8, 51.6, 46.7, 37.6, 32.5, 32.4, 20.9, 18.8 LC-MS [M+H]⁺ 484.1

Pharmacological Properties

MCH1 Receptor Radioligand Binding.

Assays were performed on membranes prepared from CHO-K1 cells expressing the human Melanin concentrating hormone receptor 1 (MCH1r). Assays were performed in a 96-well plate format in a final reaction volume of 200 μl per well. Each well contained 6 μg of membrane proteins diluted in binding buffer (50 mM Tris, 3 mM MgCl₂, 0.05% bovine serum albumin (BSA) and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at 30° C. for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a1450 Microbeta TRILUX (Wallac , Finland).

Non-specific binding was subtracted from all values determined. Maximum binding was that determined in the absence of any competitor following subtraction of the value determined for non-specific binding. Binding of compounds at various concentrations was plotted according to the equation
y=A+((B−A)/1+((C/x)ˆAD)))
and IC₅₀ estimated where
A is the bottom plateau of the curve i.e. the final minimum y value
B is the top of the plateau of the curve i.e. the final maximum y value
C is the x value at the middle of the curve. This represents the log EC50 value when A+B=100
D is the slope factor. x is the original known x values. y is the original known y values.

The compounds exemplified herein had an IC₅₀ of less than 2 μM in the abovementioned human MCHr binding assay. Preferred compounds had an activity of less than 1 μM for example an activity of more than 0.001 and less than 1 μM. For example, the following IC₅₀s were obtained for the compounds of Example 5, 0.026 μM, Example 16, 0.094 μM, Example 20, 0.56 μM, Example 32, 0.044 μM and Example 35, 0.83 μM.

Assays were also performed on membranes prepared from HEK293 cells stably expressing the rat Melanin concentrating hormone receptor 1 (MCH1r) (Lembo et al. Nature Cell Biol 1 267-271). Assays were performed in a 96-well plate format in a final reaction volume of 200 μl per well. Each well contained 5 μg of membrane proteins diluted in binding buffer (50 mM Tris, 3 mM MgCl₂, 0.05% bovine serum albumin (BSA) and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at room temperature for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a1450 Microbeta TRILUX (Wallac, Finland). For example, the following IC₅₀ was obtained for the compound of Example 6, 0.079 μM.

Compounds of the invention have the advantage that they may be more potent, more selective, more efficacious in vivo, be less toxic, be longer acting, produce fewer side effects, be more easily absorbed, be less metabolised and/or have a better pharmacokinetic profile than, or have other useful pharmacological or physicochemical properties over, compounds known in the prior art.

1. A compound of formula I 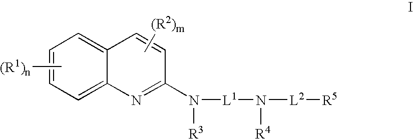
wherein
R¹ represents a C₁₋₄ alkoxy group optionally substituted by one or more fluoro, a C₁₋₄ alkyl group optionally substituted by one or more fluoro, halo, cyano, a group OSO₂C₁₋₄alkyl wherein the alkyl group is optionally substituted with one or more fluorine atoms, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
n represents 0, 1, 2 or 3;
R² represents a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H or a C₁₋₄ alkyl group;
L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively;
R⁴ represents H or a C₁₋₄ alkyl group optionally substituted by one or more of the following: fluoro or C₁₋₄ alkoxy optionally substituted by one or more fluoro;
L² represents an alkylene chain (CH₂)ₛ, in which s represents 1, 2 or 3 wherein the alkylene chain is optionally substituted by one or more of the following: fluoro or C₁₋₄ alkyl;
or L² may also represent a 5-6 membered carbocyclic ring fused to R⁵;
R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridinyl, 5H-pyrrolo[2,3-b]pyrazinyl, 1H-pyrrolo[3,2-c]pyridinyl, 1H-pyrrolo[2,3-c]pyridinyl, 1H-pyrrolo[2,3-b]pyridinyl, 1H-indazolyl, 1H-pyrrolo[3,2-h]quinolinyl, 1H-pyrrolo[3,2-b]pyridinyl, 2,1,3-benzothiadiazolyl, 2,1,3-benzoxadiazolyl, quinazolinyl or triazolyl wherein each R⁵ is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group O_{z}(CH₂)_{w}R^{z} in which z and w independently are 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro;
as well as optical isomers and racemates thereof as well as pharmaceutically acceptable salts, thereof; with the proviso that when
R¹ represents a C₁₋₄alkoxy group optionally substituted by one or more fluoro or a C₁₋₄alkyl group optionally substituted by one or more fluoro; and
n represents 0 or 1; and
R² represents a C₁₋₄alkyl group optionally substituted by one or more fluoro or a C₁₋₄alkoxy group optionally substituted by one or more fluoro; and
m represents 0 or 1; and
R³ represents H or a C₁₋₄alkyl group; and
L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group; and
L² represents an alkylene chain (CH₂), in which s represents 1, 2 or 3 wherein the alkylene chain is optionally substituted by one or more of the following: a C₁₋₄alkyl group; and
R⁵ represents aryl wherein aryl means phenyl or naphthyl each of which is optionally substituted by one or more of the following: halo, a C₁₋₄alkyl group or phenyl, or
R⁵ represents a heterocyclic group wherein the term heterocyclic group means thienyl, furyl, pyridyl, pyrrolyl, quinolinyl, indolyl, benzofuranyl or benzo[b]thienyl each of which is optionally substituted by one or more of the following: halo or a C₁₋₄alkyl group;
or L² represents a C₅₋₆cycloalkyl group which is fused to an R⁵ which is phenyl or a heteroaryl group selected from thienyl, furyl or pyrrolyl;
then R⁴ does not represent H or a C₁₋₄alkyl group; and excluding 1,4-anhydro-2,3,5-trideoxy-3-[[(3,4-dichlorophenyl)methyl]amino]-5-[(4-ethoxy-2-quinolinyl)amino]-D-erythro-pentitol.

2. A compound as claimed in claim 1 in which L¹ represents a monocyclic —(CH₂)ₚC₅₋₆(CH₂)_{q}— cycloalkyl group in which p and q are independently 0 or 1 wherein there are 3 carbon atoms between the two nitrogens bearing R³ and R⁴, respectively, wherein one of the carbons of the cycloalkyl group may be replaced by O or the group —N(R³)-L¹-, or the group L¹-N(R⁴), together represent a saturated heterocyclic ring containing from 4 to 6 carbon atoms and the nitrogen bearing R³ or R⁴ respectively.

3. A compound of formula IA 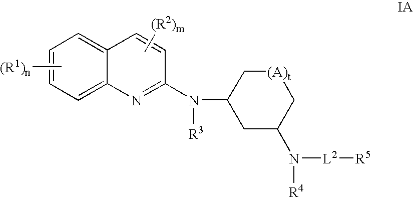
in which
R¹ represents chloro, fluoro, methoxy or a group NR^{a}R^{b} in which R^{a} and R^{b} independently represent H or a C₁₋₄alkyl group;
n represents 0, 1 or 2 and when n=1 the substituent is attached to either position 6 or 7;
R² represents a C₁₋₄alkyl group or a C₁₋₄alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R¹ and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H;
A represents CH₂ and t is 0 or 1;
R⁴ represents H;
L² represents CH₂, C(CH₃)₂ or CF₂; and
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group O_{z}(CH₂)_{w}R^{z} in which z and w independently are 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro;
as well as optical isomers and racemates thereof as well as pharmaceutically acceptable salts thereof.

4. A compound of formula IB 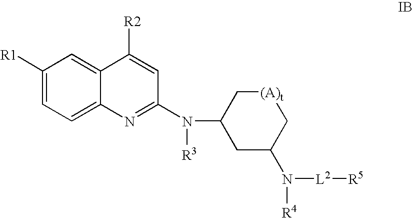
in which
R¹ represents H, methoxy, dimethylamino, chloro or fluoro;
R² represents H, a C₁₋₄alkyl group or a C₁₋₄ alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H;
A represents CH₂ and t is 0 or 1;
R⁴ represents H;
L² represents CH₂, C(CH₃)₂ or CF₂; and
R⁵ represents 2-thienyl, 3-thienyl, indol-3-yl, 2-pyrrolyl, 5-pyrimidinyl, 4-thiadiazolyl, pyrazolyl, or quinolin-2-yl each of which is optionally substituted by one or more of the following: cyano, halo, a C₁₋₄ alkyl group optionally substituted by one or more fluoro, a C₁₋₄ alkoxy group optionally substituted by one or more fluoro and in addition when R⁵ is 2-thienyl it is optionally additionally substituted by pyridyl, 2-thienyl or 3-pyrazolyl each of which is optionally substituted by halo or a C₁₋₄ alkyl group optionally substituted by one or more fluoro and when R⁵ is indol-3-yl it is optionally additionally substituted by 1-(thiazol-5-yl)methyl which is optionally substituted by halo.

5. A compound of formula IC 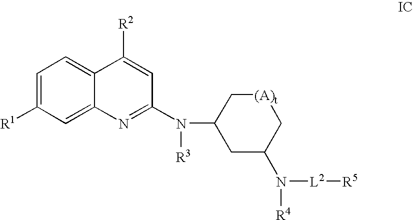
in which xR¹ represents H, methoxy, dimethylamino, chloro or fluoro;
R² represents H, a C₁₋₄alkyl group or a C₁₋₄ alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H;
A represents CH₂ and t is 0 or 1;
R⁴ represents H;
L² represents CH₂, C(CH₃)₂ or CF₂; and
R⁵ represents 2-thienyl, 3-thienyl, indol-3-yl, 2-pyrrolyl, 5-pyrimidinyl, 4-thiadiazolyl, pyrazolyl, 1H-pyrrolo[3,2-b]pyridinyl or quinolin-2-yl each of which is optionally substituted by one or more of the following: cyano, halo, a C₁₋₄ alkyl group optionally substituted by one or more fluoro, a C₁₋₄ alkoxy group optionally substituted by one or more fluoro and in addition when R⁵ is 2-thienyl it is optionally additionally substituted by pyridyl, 2-thienyl or 3-pyrazolyl each of which is optionally substituted by halo or a C₁₋₄ alkyl group optionally substituted by one or more fluoro and when R⁵ is indol-3-yl it is optionally additionally substituted by 1-(thiazol-5-yl)methyl which is optionally substituted by halo.

6. A compound as claimed in any one of claims 1 to 5 in which p is 0, q is 0 and L¹ is 1,3-cyclohexyl.

7. A compound as claimed in any one of claims 1 to 5 in which the two nitrogen atoms are in a trans orientation on the cycloalkyl ring.

8. A compound as claimed in claim 7 wherein the absolute configuration of the cycloalkyl carbon atoms to which the nitrogen atoms are attached is S, S.

9. A compound according to any one of claims 1 to 5 in which R⁵ represents one of the following:
1H-pyrrolo[3,2-c]pyridinyl;
1H-pyrrolo[2,3-b]pyridinyl;
1H-indazolyl;
1-imidazo[1,2-a]pyridinyl;
5H-pyrrolo[2,3-b]pyrazinyl;
1H-pyrrolo[3,2-b]pyridinyl;
1H-pyrrolo[3,2-h]quinolinyl;
2,1,3-benzothiadiazolyl; and
2,1,3-benzoxadiazolyl;
wherein each of these heterocycles is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or by a group O_{z}(CH₂)_{w}R^{z} in which z and w independently are 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro.

10. A compound as claimed in any one of claims 1 to 5 in which L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively; with the proviso that L¹ is not 1,4-cyclohexyl or 1,3-cyclopentyl.

11. One or more of the following compounds:
N,N-dimethyl-2-[(3-{[(5-pyridin-2-yl-2-thienyl)methyl]amino}cyclohexyl)amino]-quinoline-4-carboxamide;
(1S,3S)-N-(6-chloro-4-methylquinolin-2-yl)-N′-[(1-methyl-1H-indol-3-yl)methyl]cyclohexane-1,3-diamine;
(1S,3S)-N-(6-fluoro-4-methylquinolin-2-yl)-N′-(3-thienylmethyl)cyclohexane-1,3-diamine;
(1R,3R)-N-(6-fluoro-4-methylquinolin-2-yl)-N′-(3-thienylmethyl)cyclohexane-1,3-diamine;
(1S,3S)-N-(6-fluoro-4-methoxyquinolin-2-yl)-N′-(3-thienylmethyl)cyclohexane-1,3-diamine;
(1S,3S)-N-(6-fluoro-4-methylquinolin-2-yl)-N′-[(1-methyl-1H-indol-3-yl)methyl]cyclopentane-1,3-diamine;
N-(6-chloroquinolin-2-yl)-N′-(3-thienylmethyl)cyclohexane-1,3-diamine;
N-(6-chloroquinolin-2-yl)-N′-[(1-methyl-1H-pyrrol-2-yl)methyl]cyclohexane-1,3-diamine;
N-(6-chloroquinolin-2-yl)-N′-(quinolin-3-ylmethyl)cyclohexane-1,3-diamine;
N⁶,N⁶-dimethyl-N²-{3-[(3-thienylmethyl)amino]cyclohexyl}quinoline-2,6-diamine;
(1S,3S)-N-[(4-chloro-1-methyl-1H-pyrazol-3-yl)methyl]-N′-(6-methoxy-4-methylquinolin-2-yl) cyclopentane-1,3-diamine;
(1S,3S)-N-(6-methoxy-4-methylquinolin-2-yl)-N′-(1,2,3-thiadiazol-4-ylmethyl)cyclopentane-1,3-diamine;
(1S,3S)-N-(6-methoxy-4-methylquinolin-2-yl)-N′-[(5-pyridin-2-yl-2-thienyl)methyl]cyclopentane-1,3-diamine;
(1S,3S)-N-({1-[(2-chloro-1,3-thiazol-5-yl)methyl]-1H-indol-3-yl}methyl)-N′-(6-methoxy-4-methylquinolin-2-yl)cyclopentane-1,3-diamine;
(1S,3S)-N-(6-methoxy-4-methylquinolin-2-yl)-N′-({5-[1-methyl-5-(trifluoromethyl)-1H-pyrazol-3-yl]-2-thienyl}methyl)cyclopentane-1,3-diamine;
(1S,3S)-N-(2,2′-bithien-5-ylmethyl)-N′-(6-methoxy-4-methylquinolin-2-yl)cyclopentane-1,3-diamine;
N⁴,N⁴-dimethyl-N²-{3-[(3-thienylmethyl)amino]cyclohexyl}quinoline-2,4-diamine;
N⁴,N⁴-dimethyl-N²-[3-({[2-(phenylsulfonyl)-1,3-thiazol-5-yl]methyl}amino)-cyclohexyl]quinoline-2,4-diamine;
N²-(3-{[(2,4-dimethoxypyrimidin-5-yl)methyl]amino}cyclohexyl)-N⁴,N⁴-dimethylquinoline-2,4-diamine;
3-(6-methoxy-4-methylquinolin-2-yl)-N-methyl-N-(3-thienylmethyl)-3-azabicyclo[3.2.1]octan-8-amine;
6-methoxy-4-methyl-N-[((1R,2S)-2-{[(1-methyl-1H-indol-3-yl)methyl]amino}cyclopentyl)methyl]quinolin-2-amine;
(1S,3S)-N-(6-fluoro-4-methylquinolin-2-yl)-N′-[(1-methyl-1H-pyrrolo[2,3-b]pyridin-3-yl) methyl]cyclopentane-1,3-diamine;
(1S,3S)-3-[({3-[(7-methoxy-4-methylquinolin-2-yl)amino]cyclopentyl}amino)methyl]-1-methyl-1H-indole-6-carbonitrile;
(1S,3S)-N-(6-fluoro-4-methylquinolin-2-yl)-N′-[(1-methyl-1H-indol-2-yl)methyl]cyclopentane-1,3-diamine;
(1S,3S)-N-(6-fluoro-4-methylquinolin-2-yl)-N′-({1-[3-(trifluoromethyl)pyridin-2-yl]-1H-indol -3-yl}methyl)cyclopentane-1,3-diamine;
(1S,3S)-N-(6-fluoro-4-methylquinolin-2-yl)-N′-[(1-methyl-1H-indazol-3-yl)methyl]cyclopentane-1,3-diamine;
(1S,3S)-N-(7-methoxy-4-methylquinolin-2-yl)-N′-({1-[4-(trifluoromethyl)phenyl]-1H-pyrrol-3-yl}methyl) cyclopentane-1,3-diamine;
3-[({(1S,3S)-3-[(7-methoxy-4-methylquinolin-2-yl)amino]cyclopentyl}amino)methyl]-1-methyl-1H-indole-5-carbonitrile;
(1S,3S)-N-{[5-difluormethoxy-1H-indol-3-yl]methyl}-N′-(7-methoxy-4-methylquinolin-2-yl)cyclopentane-1,3-diamine;
(1S,2S,4R,6S)-N-(6-methoxy-4-methylquinolin-2-yl)-N′-(3-thienylmethyl)bicyclo[2.2.1 ]heptane-2,6-diamine;
(1R,2S,4S,6S)-N-(6-methoxy-4-methylquinolin-2-yl)-N′-(3-thienylmethyl)bicyclo[2.2.1]heptane-2,6-diamine;
(1S,2S,4R,6S)-N-(7-methoxy-4-methylquinolin-2-yl)-N′-[(1-methyl-1H-indol-3-yl) methyl]bicyclo[2.2.1]heptane-2,6-diamine;
6-methoxy-4-methyl-N-[(1S,2R)-2-({[(1-methyl-1H-indol-3-yl)methyl]amino}methyl)cyclopentyl]quinolin-2-amine;
(1S,3S)-N-(7-methoxy-4-methylquinolin-2-yl)-N′-[(1-methyl-1H-pyrrolo[3,2-h]quinolin-3-yl)methyl]cyclopentane-1,3-diamine;
(1S,3S)-N-(6-fluoro-4-methylquinolin-2-yl)-N′-[(1-methyl-1H-pyrrolo[2,3-c]pyridin-3-yl) methyl]cyclopentane-1,3-diamine;
(1S,3S)-N-(7-methoxy-4-methylquinolin-2-yl)-N′-[(1-methyl-1H-pyrrolo[3,2-b]pyridin-3-yl) methyl]cyclopentane-1,3-diamine;
(1S,3S)-N-(6-fluoro-4-methylquinolin-2-yl)-N′-(imidazo[1,2-a]pyridin-3-ylmethyl) cyclopentane-1,3-diamine;
(1S,3S)-N-{[5-(Benzyloxy)-1-methyl-1H-indol-3-yl]methyl}-N′-(7-methoxy-4-methylquinolin-2-yl) cyclopentane-1,3-diamine;
(1S,3S)-N-(7-Methoxy-4-methylquinolin-2-yl)-N′-[3-(trifluoromethoxy)benzyl]-cyclohexane-1,3-diamine;
(1S,3S)-N-(2,1,3-Benzothiadiazol-4-ylmethyl)-N′-(7-methoxy-4-methylquinolin-2-yl) cyclohexane-1,3-diamine;
(1S,3S)-N-[(1,3-Dimethyl-1H-pyrazol-4-yl)methyl]-N′-(7-methoxy-4-methylquinolin-2-yl)cyclohexane-1,3-diamine; and
(1S,3S)-N-(2-Bromo-4-methoxybenzyl)-N′-(7-methoxy-4-methylquinolin-2-yl)cyclohexane-1,3-diamine;
and pharmaceutically acceptable salts thereof.

12. (canceled)

13. A pharmaceutical formulation comprising a compound as defined in any one of claims 1 to 5 or claim 11 and a pharmaceutically acceptable adjuvant, diluent or carrier.

14. (canceled)

15. A method of treating obesity, a psychiatric disorder, anxiety, an anxio-depressive disorder, depression, bipolar disorder, ADHD, a cognitive disorder, a memory disorder, schizophrenia, epilepsy, a neurological disorder and a pain related disorder, comprising administering a pharmacologically effective amount of a compound as claimed in any one of claims 1 to 5 or claim 11 to a patient in need thereof.

16. (canceled)

17. A process for the preparation of a compound of formula I 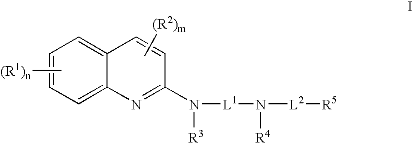
comprising reacting a compound of formula II 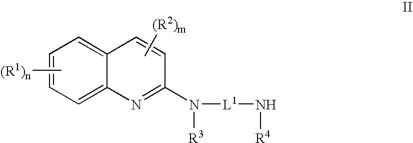
in which
R¹ represents a C₁₋₄alkoxy group optionally substituted by one or more fluoro, a C₁₋₄ alkyl group optionally substituted by one or more fluoro, halo, cyano, a group OSO₂C₁₋₄ alkyl wherein the alkyl group is optionally substituted with one or more fluorine atoms, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R in which R^{c} and R^{d} independently represent H or a C₁₋₄alkyl group or R^{c} and R^{d} together with the nitrogen atom to which they are attached represent a saturated 3 to 7 membered heterocyclic ring;
n represents 0, 1, 2 or 3;
R² represents a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H or a C₁₋₄ alkyl group;
L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively;
R⁴ represents H or a C₁₋₄ alkyl group optionally substituted by one or more of the following: fluoro or C₁₋₄ alkoxy optionally substituted by one or more fluoro;
with a compound of formula III
R⁵-L^{2′}=O III
in which R⁵ is as previously defined and L^{2′} represents a group which after reaction of compounds II and III gives L² on reduction, under reductive alkylation conditions.

18. A compound of formula II 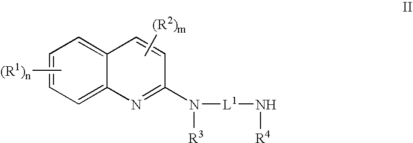
in which
R¹ represents a C₁₋₄ alkoxy group optionally substituted by one or more fluoro, a C₁₋₄ alkyl group optionally substituted by one or more fluoro, halo, cyano, a group OSO₂C₁₋₄alkyl wherein the alkyl group is optionally substituted with one or more fluorine atoms, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
n represents 0, 1, 2 or 3;
R² represents a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H or a C₁₋₄ alkyl group;
L¹ represents a (CH₂)ₚC₃₋₁₀ cycloalkyl(CH₂)_{q} group in which p and q are independently selected from 0 and 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively; and
R⁴ represents H or a C₁₋₄ alkyl group optionally substituted by one or more of the following: fluoro or C₁₋₄ alkoxy optionally substituted by one or more fluoro.

19. A compound selected from one or more of:
(1S,3S)-Dibenzyl-cyclohexane-1,3-diylbiscarbamate; and
(1S,3S)-Cyclohexane-1,3-diamine dihydrochloride.

20. A method of treating obesity, type II diabetes, or Metabolic syndrome comprising administering a pharmacologically effective amount of a compound as claimed in any one of claims 1 to 5 or claim 11 to a patient in need thereof.

21. A method of preventing type II diabetes comprising administering a pharmacologically effective amount of a compound as claimed in any one of claims 1 to 5 or claim 11 to a patient in need thereof.
